(19) United States
(12) Patent Application Publication
(10) Pub. No.: US 2025/0129146 A1
(43) Pub. Date: Apr. 24, 2025

(54) COMBINED CANCER THERAPY WITH AN EPITHELIAL CELL ADHESION MOLECULE (EPCAM) INHIBITOR AND A WNT INHIBITOR

(71) Applicant: ACADEMIA SINICA, Taipei City (TW)

(72) Inventors: Han-Chung WU, Taipei City (TW); Sushree Shankar PANDA, Odisha (IN)

(73) Assignee: ACADEMIA SINICA, Taipei City (TW)

(21) Appl. No.: 18/572,564

(22) PCT Filed: Jun. 24, 2022
(86) PCT No.: PCT/US2022/034885

§ 371 Date: Dec. 20, 2023

Related U.S. Application Data
(60) Provisional application No. 63/215,036, filed on Jun. 25, 2021.

(51) Int. Cl. C07K 16/22; A61K 31/497; A61K 39/00; A61P 35/00

(52) U.S. Cl. CPC C07K 16/22; A61K 31/497; A61P 35/00; A61K 2039/505; C07K 2317/34; C07K 2317/565; C07K 2317/73; C07K 2317/76

(57) ABSTRACT

The present invention relates to combined therapy of cancer using an epithelial cell adhesion molecule (EpCAM) inhibitor and a Wnt signaling inhibitor. Specifically, the EpCAM inhibitor is an antibody which is directed to an extracellular domain (EpEX) of EpCAM. The combined therapy is effective in inducing apoptosis of cancer cells, inhibiting cancer sternness properties, tumor progression and/or metastasis, and/or prolonging survival of a cancer patient.

### RELATED APPLICATIONS

This application claims the benefit of U.S. provisional application No. 63/215,036, filed Jun. 25, 2021 under 35 U.S.C. § 119, the entire content of which is incorporated herein by reference.

### TECHNOLOGY FIELD

The present invention relates to combined therapy of cancer using an epithelial cell adhesion molecule (EpCAM) inhibitor and a Wnt inhibitor. Specifically, the EpCAM inhibitor is an antibody against an extracellular domain (EpEX) of EpCAM. The combined therapy is effective in inducing apoptosis of cancer cells, inhibiting cancer stemness properties, tumor progression and/or metastasis, and/or prolonging survival of a cancer patient.

### BACKGROUND OF THE INVENTION

Epithelial cell adhesion molecule (EpCAM; also known as CD326) is highly expressed in many cancer types including colorectal cancer (CRC). Unlike its cell adhesion functions in healthy epithelial cells, the protein is activated by cleavage at the cellular membrane releasing the extracellular domain (EpEX) and intracellular domain (EpICD) that fuel tumor progression by participating in proliferation, epithelial to mesenchymal transition (EMT), sternness and differentiation (Chen et al., 2020; Gires et al., 2009; Gires et al., 2020; Liang et al., 2018; Lin et al., 2012; Maetzel et al., 2009; Sankpal et al., 2017). In this regard, EpEX was reported to directly bind to EGFR, stimulating EGFR phosphorylation and its downstream signaling including stabilization of PD-L1 (Chen et al., 2020; Liang et al., 2018; Pan et al., 2018). Importantly, EpCAM has been shown to be a potent cancer stem cell (CSC) antigen; its exact role however is poorly understood (Gires et al., 2009; Gires et al., 2020; Lin et al., 2012). In this context, one pathway known to play a central role in CSC pathobiology is the Wnt-β-Catenin signaling that is involved in promoting several malignancy-associated features, such as tumorigenic potential, tumor plasticity and drug resistance making the pathway an intriguing therapeutic target in cancer (Kahn, 2014; Nusse and Clevers, 2017). It has been suggested that targeting the CSC population might be a beneficial therapeutic strategy; however, conclusive identification of CSC populations still poses a significant challenge (Batlle and Clevers, 2017). In order to target CSCs in cancer therapy, it may be possible to block activation of the Wnt pathway by targeting factors in the tumor microenvironment that signal to CSCs (Batlle and Clevers, 2017; Nusse and Clevers, 2017; Zhan et al., 2017).

EpCAM could be one such mediator of Wnt signaling in CSCs since EpICD is a well-studied factor that promotes cell motility, proliferation, survival and metastasis (Gires et al., 2009; Gires et al., 2020; Liang et al., 2018; Lin et al., 2012; Park et al., 2016). More importantly, soluble EpICD is known to form a multi-protein nuclear complex with the β-Catenin and a scaffolding protein called Four and one-half LIM domains protein 2 (FHL2), translocates to the nucleus, where it associates with T-Cell Factor (TCF) or Lymphoid Enhancer Factor 1 (LEF-1) thus may transcribe Wnt target genes (Maetzel et al., 2009; Park et al., 2016; Ralhan et al., 2010). However, it is unknown whether EpEX is somehow coordinated with the Wnt pathway.

For colorectal cancer (CRC) patients, high EpCAM expression suggests poor outcomes, corresponding with the known critical involvement of EpICD in CRC cell function (Chen et al., 2020; Kim et al., 2016; Liang et al., 2018; Lin et al., 2012; Seeber et al., 2016; Wang et al., 2016). Moreover, EpCAM potentiates CRC stem cell oncogenicity via its ability to stimulate reproduction and phenotypic heterogeneity of parental tumorigenic cells. In a mouse model, EpCAM^{high}/CD44⁺ cells not only displayed high tumorigenicity but also successfully differentiated into several subpopulations indicative of their sternness (Boesch et al., 2018; Dalerba et al., 2007). In fact, nuclear translocation of the EpICD-β-Catenin complex is known to upregulates transcription of reprogramming genes, such as Oct4, Sox2 and c-Myc conferring self-renewal ability to CRC cells as well as activation of EMT-inducing genes, such as Snail1, Slug and Twist (Lin et al., 2012). Therefore, a further understanding of the functional repertoire of EpCAM may shed light on how to target CRC stem cells.

In cancer, since EpICD functions in a complex with β-Catenin, Wnt signaling could be implicated in EpCAM activity (Liang et al., 2018; Maetzel et al., 2009; Park et al., 2016; Ralhan et al., 2010). Notably, Wnt signaling components are abundant and aberrantly regulated in CRC, with Wnt-associated proteins exerting major effects on cancer cell sternness, self-renewal and heterogeneity (Batlle and Clevers, 2017; de Sousa e Melo et al., 2017; Kozar et al., 2013; Nusse and Clevers, 2017; Schepers et al., 2012). Additionally, almost 80% of all colorectal tumors carry loss-of-function mutations in the gene for Adenomatous polyposis coli (APC), and around 5% of CRC tumors carry activating mutations in β-Catenin (Cancer Genome Atlas, 2012; Morin et al., 1997). It remains controversial whether CRC cells harboring such mutations require external Wnt ligands to drive the signaling; however, Voloshanenko et al. reported that regardless of Wnt-activating mutations, Wnt secretion and its interaction with receptors is required to drive and sustain high-level Wnt activity (Voloshanenko et al., 2013). Similarly, it was also conclusively shown that phosphorylation of β-Catenin at S33, S37 and T41 can occur in cells harboring mutations at the priming phosphorylation site, S45, sensitizing the cells to Wnt ligands (Wang et al., 2003). Therefore, one strategy to target the Wnt pathway could be to impede Wnt activation by inhibiting porcupine, an o-acyl-transferase required for the palmitoylation of Wnt proteins (Nusse and Clevers, 2017). In addition, Wnt activity is governed by extrinsic cues in the tumor microenvironment thus was found to functionally determine CRC cell stemness, independent of APC or β-Catenin mutations (Vermeulen et al., 2010). Therefore, in order to best target CRC tumors, it may be beneficial to inhibit stemness properties by targeting essential intracellular signaling events as well as extrinsic cues from the microenvironment to CRC stem cells (Batlle and Clevers, 2017; Nusse and Clevers, 2017; Vermeulen et al., 2010).

### SUMMARY OF THE INVENTION

Disclosed here is combined use of an epithelial cell adhesion molecule (EpCAM) inhibitor and a Wnt signaling inhibitor for treating cancer. Specifically, the EpCAM inhibitor is an antibody against an extracellular domain (EpEX) of EpCAM. The combined therapy is effective in inducing apoptosis of cancer cells, inhibiting cancer stemness properties, tumor progression and/or metastasis, and/or prolonging survival of a cancer patient.

In one aspect, the present invention provides a method for treating cancer, comprising administering to a subject in need thereof
(i) an effective amount of a first inhibitory agent that inhibits the activation of EpCAM signaling; and
(ii) an effective amount of a second inhibitory agent that inhibits the activation of Wnt signaling.

In some embodiments, the first inhibitory agent reduces production (or release) of EpEX and/or blocks binding of EpEX to a Wnt receptor.

In some embodiments, the second inhibitory agent blocks binding of a Wnt ligand to a Wnt receptor protein. Specifically, the Wnt ligand is not EpEX.

In some embodiments, the first inhibitory agent is an antibody directed to EpEX (anti-EpEX antibody) or an antigen-binding fragment thereof.

In some embodiments, the anti-EpEX antibody as described herein specifically binds to epidermal growth factor (EGF)-like domains I and II. In certain examples, the anti-EpEX antibody as described herein has a specific binding affinity to an epitope within the sequence of CVCENYKLAVN (aa 27 to 37) (SEQ ID NO: 20) located in the EGF-like domain I, and KPEGALQNNDGLYDPDCD (aa 83 to 100) (SEQ ID NO: 19) located in the EGF-like domain II.

In some embodiments, the antibody or antigen-binding fragment comprises
(a) a heavy chain variable region (VH) which comprises a heavy chain complementary determining region 1 (HC CDR1) comprising the amino acid sequence of SEQ ID NO: 2, a heavy chain complementary determining region 2 (HC CDR2) comprising the amino acid sequence of SEQ ID NO: 4, and a heavy chain complementary determining region 3 (HC CDR3) comprising the amino acid sequence of SEQ ID NO: 6; and
(b) a light chain variable region (VL) which comprises a light chain complementary determining region 1 (LC CDR1) comprising the amino acid sequence of SEQ ID NO: 9, a light chain complementary determining region (LC CDR2) comprising the amino acid sequence of SEQ ID NO: 11, and a light chain complementary determining region 3 (LC CDR3) comprising the amino acid sequence of SEQ ID NO: 13.

In some embodiments, the VH comprises the amino acid sequence of SEQ ID NO: 15, and/or the VL comprises the amino acid sequence of SEQ ID NO: 16.

In some embodiments, the first inhibitory agent is effective in inhibiting β-Catenin signaling.

In some embodiments, the second inhibitory agent is a porcupine inhibitor.

In some embodiments, the method of the present invention is effective in inducing apoptosis of cancer cells.

In some embodiments, the method of the present invention is effective in inhibiting cancer sternness properties, tumor progression and/or metastasis.

In some embodiments, the method of the present invention is effective in prolonging survival of the subject.

In some embodiments, the cancer to be treated is selected from the group consisting of lung cancer, brain cancer, breast cancer, cervical cancer, colon cancer, gastric cancer, head and neck cancer, kidney cancer, leukemia, liver cancer, ovarian cancer, pancreatic cancer, prostate cancer, skin cancer and testicular cancer.

In another aspect, the present invention provides a kit of a pharmaceutical composition comprising
(i) a first inhibitory agent that inhibits the activation of EpCAM signaling; and
(ii) a second inhibitory agent that inhibits the activation of Wnt signaling.

Also provided in the present invention is use of a combination of (i) a first inhibitory agent that inhibits the activation of EpCAM signaling and (ii) a second inhibitory agent that inhibits the activation of Wnt signaling for manufacturing a medicament or kit for treating cancer.

The details of one or more embodiments of the invention are set forth in the description below. Other features or advantages of the present invention will be apparent from the following detailed description of several embodiments, and also from the appending claims.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIGS. 1A to 1D. EpCAM is correlated with active β-Catenin in CRC patient samples. (FIG. 1A) IHC staining for EpCAM and active β-Catenin in various stages of CRC (Scale bar: 100 μm). Quantification of expression intensities in samples from 120 patients for (FIG. 1B) EpCAM and (FIG. 1C) active β-Catenin expression. (FIG. 1D) EpCAM correlation with active β-Catenin in 120 patient samples showing Pearson correlation coefficient r. Data were analyzed using one-way ANOVA followed by Bonferroni correction and error bars represent±SD of the mean. *p<0.05, **p<0.01, ***p<0.001, ****p<0.0001.

FIGS. 2A to 2K. EpEX promotes nuclear translocation of β-catenin and related biological functions. (FIG. 2A) IFS shows nuclear β-Catenin with indicated treatment; quantification of nuclear β-Catenin from 50 cells in each group is included (Scale bar: 10 am). (FIG. 2B) Western blot analysis shows active β-Catenin expression in various cell fractions with indicated treatment and (FIG. 2C) Corresponding TCF activity (%) as indicated in HCTT16 cells. Indicated treatment shows (FIG. 2D) TCF activity (%) in SW620 cells (FIG. 2E) Western blot analysis showing Axin2 expression and (FIG. 2F) corresponding mRNA expression in HT29 cells. (FIG. 2G) IFS showing nuclear β-Catenin with indicated treatment. Quantification of nuclear β-Catenin from 50 cells in each group (Scale bar: 10 μm) and corresponding (FIG. 2H) Western blot confirming nuclear β-Catenin levels (quantification of the band intensities from three independent experiments) in HCT116 cells. (FIG. 2I) TCF activity (%) in Colo205 cells (FIG. 2J) Western blot analysis showing Axin2 expression and (FIG. 2K) relative mRNA expression in Colo205 cells with indicated treatment. Data were analyzed using one-way ANOVA or two-way ANOVA (C) followed by Bonferroni correction and error bars represent ±SD of the mean. *p<0.05, **p<0.01, ***p<0.001, ****p<0.0001. Ctrl: control.

FIGS. 3A to 3G. EpEX stimulates nuclear translocation of β-catenin independent of EpICD. (FIG. 3A) Western blot analysis showing nuclear β-Catenin in EpCAM-knockdown HCT116 cells; quantification of band intensities from three independent experiments. (FIG. 3B) Immunofluorescence and (FIG. 3C) western blot analysis showing nuclear β-Catenin in EpCAM-KO HCTT16 cells and with EpEX treatment (FIG. 3D) Immunofluorescence and (FIG. 3E) western blot analysis showing nuclear β-Catenin with indicated treatment in HCTT16 cells (FIG. 3F, FIG. 3G) corresponding TCF (%) activity with indicated treatment in HCT116 cells. (All confocal images: Scale bar 10 μm and quantifications of nuclear β-Catenin from 30 cells in each group). Statistics were performed using one-way ANOVA followed by Bonferroni correction and error bars represent ±SD of the mean. *p<0.05, **p<0.01, ***p<0.001, * ***p<0.0001. Ctrl: control, KO: Knock-out

FIGS. 4A to 4E. EpEX and Wnt proteins coordinately regulate nuclear translocation of β-Catenin and related biological function. (FIG. 4A) IFS (Scale bar 10 μm and quantifications of nuclear β-Catenin from 30 cells in each group included), (FIG. 4B) western blot analyses showing nuclear β-Catenin, (FIG. 4C) Corresponding TCF (%) activity, (FIG. 4D) Wnt-target Axin2 expression and (FIG. 4E) relative Axin2 mRNA expression in HCT116 cells with indicated treatment. Statistics were performed using one-way ANOVA followed by Bonferroni correction and error bars represent±SD of the mean. *p<0.05, **p<0.01, ***p<0.001, ****p<0.0001. Ctrl: control.

FIG. 5A to FIG. 5C. Combinatorial inhibition of EpCAM and Wnt signaling abolishes Wnt related function. (FIG. 5A) Corresponding TCF (%) activity, (FIG. 5B) Wnt-target Axin2 expression and (FIG. 5C) relative Axin2 mRNA expression in HCT116 cells with indicated treatment. Statistics were performed using one-way ANOVA followed by Bonferroni correction and error bars represent±SD of the mean. *p<0.05, **p<0.01, ***p<0.001, ****p<0.0001. Ctrl: control.

FIGS. 6A to 6H. hEpAb2-6 attenuates nuclear translocation of β-Catenin, limits cancer stemness and induces apoptosis. (FIG. 6A) Immunofluorescence shows nuclear β-Catenin in HT29 cells with indicated antibody or inhibitor treatment; quantification of nuclear β-Catenin from 30 cells in each group (Scale bar: 10 μm). (FIG. 6B) Western blot analyses show nuclear and total β-Catenin after indicated antibody or inhibitor treatments in HT29 cells; (FIG. 6C) TCF activity is shown. (FIG. 6D) Tumorsphere and colony formation assay, (FIG. 6E) sphere number and (FIG. 6F) colony density (5×10³ cells were seeded for each case) after indicated inhibitor and antibody treatments. (G-H) AnnexinV apoptosis assay with indicated treatments; quantification of apoptotic cells from three independent experiments in HCT116 cells. Statistics were performed using one-way ANOVA followed by Bonferroni correction; error bars represent±SD of the mean. *p<0.05, **p<0.01, ***p<0.001. Ctrl: control.

FIGS. 7A to 7E. Targeting EpCAM and Wnt signaling attenuates stemness in CRC. (FIG. 7A, FIG. 7B, FIG. 7C) Western and qPCR showing knocked out (KO) or forced expression (OE) of EpCAM in indicated cells lines. (FIG. 7D) Growth curve comparison in EpCAM-KO HT29 cells. (FIG. 7E) Tumorsphere formation with indicated treatment in HT29 cells. Data were analyzed using one-way ANOVA or two-way ANOVA (D) followed by Bonferroni correction and error bars represent ±SD of the mean. *p<0.05, **p<0.01, ***p<0.001, ****p<0.0001. Ctrl: control.

FIGS. 8A to 8N. EpEX/EpCAM and Wnt signaling cooperatively regulate cancer stemness. (FIG. 8A) Growth curve comparison in indicated HCT116 and (FIG. 8B) CT26 cells. (FIG. 8C, FIG. 8D, FIG. 8E, FIG. 8F) Comparison of tumor sizes and progression in vivo; 10³ Ctrl and EpCAM-KO HCT116 cells were subcutaneously transplanted in NSG mice (n=6 for each cell line). (FIG. 8G) In vitro regeneration assay with control and EpCAM-KO HT29 cells. (FIG. 8H) Tumor sphere formation and (FIG. 8I) sphere count with indicated treatment in HCT116 cells. Colony (FIG. 8J) formation and (FIG. 8K) density (5×10³ cells seeded) with indicated treatment in HT29 cells. (FIG. 8L) Sphere and colony formation, (FIG. 8M) colony densities and (FIG. 8N) Sphere number with indicated treatment in SW620 cells (1×10³ cells seeded for both assays). Data were analyzed using one-way ANOVA or two-way ANOVA (FIG. 8A, FIG. 8B, FIG. 8D) followed by Bonferroni correction and error bars represent ±SD of the mean. *p<0.05, **p<0.01, ***p<0.001, ****p<0.0001. Ctrl: control, KO: EpCAM-Knock-out, OE: EpCAM forced expression.

FIGS. 9A to 9I. EpEX interacts with Wnt receptors thus induces Wnt signaling. (FIG. 9A, FIG. 9B) Co-Immunoprecipitation (Co-IP) of affinity cross-linked EpEX/Wnt receptor proteins yielded complexes in HCT116 cells. ELISA showing either (FIG. 9C) EpEX alone or (FIG. 9D) EpEX incubated with indicated antibody complex binding to purified Wnt receptor-GST fusion protein-coated plates. (FIG. 9E) Western blot analysis showing phosphorylation of LRP5/6 with indicated treatment in HCTT16 cells and quantified band intensities from three independent experiments. (FIG. 9F) HEK293 cells were transfected with EGF-domain (1/11)-deleted mutant EpCAM-V5 plasmid. IP of affinity cross-linked mutant EpCAM-V5/Wnt receptor proteins yielded complexes that blotted with respective receptor antibody. HT29 cells were treated with EGF-domain (1/11)-deleted mutant EpEX proteins when (FIG. 9G) Western blotting indicating phosphorylation of LRP5/6 and quantified band intensities from at least three independent experiments and (FIG. 9H) IFS showing nuclear translocation Q-Catenin in HCTT16 cells with mutant EpEX treatment; quantification of nuclear β-Catenin from 50 cells in each group (scale bar 10 μM). (FIG. 9I) Western blotting showing inhibition of phosphorylation of LRP5/6 with indicated treatment and quantified band intensities from three independent experiments in SW620 cells. Data were analyzed using one-way ANOVA followed by Bonferroni correction and error bars represent ±SD of the mean. *p<0.05, **p<0.01, ***p<0.001, ****p<0.0001. Ctrl: control, GST: Glutathione S-transferase, pAb: polyclonal antibody.

FIGS. 10A to 10G. EpEX and Wnt proteins activate TACE and γ-secretase enzymes. TACE activity in (FIG. 10A) HCT116 cells and (FIG. 10B) H29 cells, and γ-secretase activity in (FIG. 10C) HCT116 cells and (FIG. 10D) H29 cells after indicated treatments. (FIG. 10E) Western blot analysis showing levels of phosphorylated TACE and PS2 in HCT116 cells after indicated treatments. The effects of (FIG. 10F) BIO and (FIG. 10G) PF-670462 treatments on phosphorylated TACE and PS2; band intensities from at least three independent experiments were quantified. Data were analyzed using one-way ANOVA followed by Bonferroni multiple comparisons. Error bars represent±SD of the mean. *p<0.05, **p<0.01, ***p<0.001, ****p<0.0001. Ctrl: control.

FIGS. 11A to 11M. EpICD upregulates transcription of Wnt receptor proteins and stemness factors. (FIG. 11A) Wnt receptor protein expression in EpCAM knockdown H29 cells and (FIG. 11B) relative mRNA expression. (FIG. 11C) Western blot analysis showing Wnt receptor protein expression in EpCAM-KO and transfected with EpCAM plasmid in HCT116 cells and quantified band intensities from three independent experiments and (FIG. 11D) corresponding relative mRNA expression. (FIG. 11E) Wnt receptor protein expression in HT29 cells with overnight DAPT treatment and quantified band intensities from three independent experiments and corresponding (FIG. 11F) relative mRNA expression. Wnt receptor promoter activity after transfection of EpCAM plasmid and DAPT treatment in (FIG. 11G) HCT116 cells and (FIG. 11H) EpCAM-KO HT29 cells and (FIG. 11I) SW620 cells. (FIG. 11J) Western blot analysis showing Wnt receptor expressions after indicated treatment in HCT116 cells and quantified band intensities from three independent experiments (FIG. 11K) relative mRNA expression. (FIG. 11L) Western blot analysis showing mentioned sternness factors expressions with indicated treatments in HT29 cells and quantified band intensities from three independent experiments; (FIG. 11M) relative mRNA expressions. Data were analyzed using one-way ANOVA followed by Bonferroni correction and error bars represent ±SD of the mean. *p<0.05, **p<0.01, ***p<0.001, ****p<0.0001. Ctrl: control, Trans: Transfection.

FIGS. 12A to 12F. EpICD promotes transcription of Wnt receptors. (FIG. 12A) Wnt receptor protein expression in EpCAM-KO HCT116 cells and (FIG. 12B) corresponding mRNA expressions. (FIG. 12C) Cell morphology comparison among EpCAM-KO HCT116 cells with or without transfected EpCAM plasmid. (FIG. 12D) Western blot analysis showing Wnt receptor protein expression with DAPT treatment in HCT116 cells. (FIG. 12E) Wnt-receptor promoter plasmid constructions with luciferase reporter. Wnt receptor promoter activity after transfection of EpCAM plasmid and overnight DAPT treatment in (FIG. 12F) EpCAM-KO HCT116 cells. Data were analyzed using one-way ANOVA followed by Bonferroni correction and error bars represent±SD of the mean. *p<0.05, **p<0.01, ***p<0.001. Ctrl: control; KO: Knockout; PM: Promoter; LUC: Luciferase.

FIG. 13A to 13F. EpEX and Wnt proteins collaboratively promote Wnt receptor and stemness factor expressions. (FIG. 13A) Western blot analysis showing Wnt receptor protein expressions with indicated treatment in HCT116 cells and (FIG. 13B) relative mRNA expression. (FIG. 13C) Western blot analysis showing indicated sternness factor expressions in EpCAM knockdown HCT116 cells; (FIG. 13D) relative mRNA expressions. (FIG. 13E) Western blot analysis showing indicated sternness factor expressions HT29 cells with indicated treatment, (FIG. 13F) relative mRNA expressions. Data were analyzed using one-way ANOVA followed by Bonferroni correction and error bars represent ±SD of the mean. *p<0.05, **p<0.01, ***p<0.001. Ctrl: control.

FIGS. 14A to 14F. EpAb2-6 and LGK974 coordinately inhibit tumor progression. (FIG. 14A) Annexin V apoptosis assay with indicated treatment in SW620 cells, and (FIG. 14B) quantification of apoptotic cell counts from three independent experiments. (FIG. 14C) Kaplan-Meier survival plot showing animal survival for the metastatic model after indicated treatment. (FIG. 14D) Bioluminescence indicating tumor progression in orthotopic animal models (Day 0=72 hr post-transplantation) (FIG. 14E) Quantification of luminescence (FIG. 14F) Kaplan-Meier survival plot showing animal survival for the orthotopic model. Data were analyzed using one-way ANOVA or two-way ANOVA (FIG. 14E) followed by Bonferroni correction and error bars represent ±SD of the mean. *p<0.05, **p<0.01, ***p<0.001, ****p<0.0001. Ctrl: control.

FIGS. 15A to 15E. EpCAM and Wnt signaling collegially confer tumor progression thus their inhibition induce cancer cell apoptosis and deter metastasis. (FIG. 15A, FIG. 15B) AnnexinV apoptosis assay with indicated treatments; quantification of apoptotic cells from three independent experiments in HCT116 cells. (FIG. 15C) Treatment schedule of both metastatic and orthotopic animal models of CRC (HCT116 cells). (FIG. 15D) Animal bodyweight comparison after indicated treatments in metastatic model (FIG. 15E) Autopsies revealed mouse deaths were due to tumors metastasize to various organs in metastasis model. (F) Mouse bodyweight comparison in the orthotopic animal model. Data were analyzed using one-way ANOVA followed by Bonferroni correction and error bars represent SD of the mean. *p<0.05, **p<0.01, ***p<0.001. Ctrl: control.

FIG. 16. Summary of EpCAM induces Wnt signaling promoting stemness in CRC, thus the combined inhibition by EpAb2-6 and a porcupine inhibitor could suppress cancer sternness and improve CRC treatment.

FIGS. 17A to 17B. Sequence features and domains of human EpCAM. (FIG. 17A) Full length of human EpCAM containing 314 amino acid residues (SEQ ID NO: 17). (FIG. 17B) identification of domains of EpCAM where the EpEX domain includes EGF I domain (aa 27-59) covering VGAQNTVIC (aa 51 to 59, SEQ ID NO: 18) and EGF II domain (aa 66-135) covering KPEGALQNNDGLYDPDCD (aa 83 to 100, SEQ ID NO: 19) with the LYD motif (aa 94-96).

FIGS. 18A to 18G. EpAb2-6 binds to both EGF-like domain I and II of EpCAM. HEK293T cells were transfected with full length or EGF like-domain deletion mutant EpCAM-V5. Antibody binding was assessed by (FIG. 8A) Western blotting, (FIG. 8B) flow cytometry, and (FIG. 8C) immunofluorescence. (FIG. 8D) EpCAM mutants were constructed with amino acid substitutions in the EGF-I (Y32A) and EGF-II (L94A, Y95A, or D96A) domains. EpCAM wild-type and mutant proteins were expressed in HEK293T cells. Binding of MT201, EpAb2-6 and EpAb23-1 to EpCAM wild-type and mutants were evaluated by (FIG. 8E) immunofluorescence, (FIG. 8F) flow cytometry, and (FIG. 8G) cellular ELISA. All data are presented as mean±SEM. *, p<0.05; **, p<0.01.

FIG. 19. The amino acid sequences of EpAb2-6, in which a VH (SEQ ID NO: 15) comprising HC CDR1 of SEQ ID NO: 2, HC CDR2 of SEQ ID NO: 4, and HC CDR3 of SEQ ID NO: 6; and a VL (SEQ ID NO: 16) comprising LC CDR1 of SEQ ID NO: 9, LC CDR2 of SEQ ID NO: 11, and HC CDR3 of SEQ ID NO: 13.

### DETAILED DESCRIPTION OF THE INVENTION

The following description is merely intended to illustrate various embodiments of the invention. As such, specific embodiments or modifications discussed herein are not to be construed as limitations to the scope of the invention. It will be apparent to one skilled in the art that various changes or equivalents may be made without departing from the scope of the invention.

In order to provide a clear and ready understanding of the present invention, certain terms are first defined. Additional definitions are set forth throughout the detailed description. Unless defined otherwise, all technical and scientific terms used herein have the same meanings as is commonly understood by one of skill in the art to which this invention belongs.

As used herein, the singular forms “a”, “an”, and “the” include plural referents unless the context clearly dictates otherwise. Thus, for example, reference to “a component” includes a plurality of such components and equivalents thereof known to those skilled in the art.

The term “comprise” or “comprising” is generally used in the sense of include/including which means permitting the presence of one or more features, ingredients or components. The term “comprise” or “comprising” encompasses the term “consists” or “consisting of.”

As used herein, the term “polypeptide” refers to a polymer composed of amino acid residues linked via peptide bonds. The term “protein” typically refers to relatively large polypeptides. The term “peptide” typically refers to relatively short polypeptides (e.g., containing up to 100, 90, 70, 50, 30, 20 or 10 amino acid residues).

As used herein, the term “approximately” or “about” refers to a degree of acceptable deviation that will be understood by persons of ordinary skill in the art, which may vary to some extent depending on the context in which it is used.

Specifically. “approximately” or “about” may mean a numeric value having a range of 10% or 5% or 3% around the cited value.

As used herein, the term “substantially identical” refers to two sequences having 80% or more, preferably 85% or more, more preferably 90% or more, even more preferably 95% or more homology.

As used herein, the term “antibody” (interchangeably used in plural form, antibodies) means an immunoglobulin molecule having the ability to specifically bind to a particular target antigenic molecule. As used herein, the term “antibody” includes not only intact (i.e. full-length) antibody molecules but also antigen-binding fragments thereof retaining antigen binding ability e.g. Fab, Fab′, F(ab′)2 and Fv. Such fragments are also well known in the art and are regularly employed both in vitro and in vivo. The term “antibody” also includes chimeric antibodies, humanized antibodies, human antibodies, diabodies, linear antibodies, single chain antibodies, multispecific antibodies (e.g., bispecific antibodies) and any other modified configuration of the immunoglobulin molecule that comprises an antigen recognition site of the required specificity, including amino acid sequence variants of antibodies, glycosylation variants of antibodies, and covalently modified antibodies.

An intact or complete antibody comprises two heavy chains and two light chains. Each heavy chain contains a variable region (VH) and a first, second and third constant regions (C_{H}1, C_{H}2 and C_{H}3); and each light chain contains a variable region (VL) and a constant region (CL). The antibody has a “Y” shape, with the stem of the Y consisting of the second and third constant regions of two heavy chains bound together via disulfide bonding. Each arm of the Y includes the variable region and first constant region of a single heavy chain bound to the variable and constant regions of a single light chain. The variable regions of the light chains and those of heavy chains are responsible for antigen binding. The variables regions in both chains are responsible for antigen binding generally, each of which contain three highly variable regions, called the complementarity determining regions (CDRs); namely, heavy (H) chain CDRs including HC CDR1, HC CDR2, HC CDR3 and light (L) chain CDRs including LC CDR1, LC CDR2, and LC CDR3. The three CDRs are franked by framework regions (FR1, FR2, FR3, and FR4), which are more highly conserved than the CDRs and form a scaffold to support the hypervariable regions. The constant regions of the heavy and light chains are not responsible for antigen binding, but involved in various effector functions. Depending on the antibody amino acid sequence of the constant domain of its heavy chains, immunoglobulins can be assigned to different classes. There are five major classes of immunoglobulins: IgA, IgD, IgE, IgG, and IgM. The heavy-chain constant domains that correspond to the different classes of immunoglobulins are called alpha, delta, epsilon, gamma, and mu, respectively.

As used herein, the term “antigen-binding fragment” or “antigen-binding domain” refers to a portion or region of an intact antibody molecule that is responsible for antigen binding. An antigen-binding fragment is capable of binding to the same antigen to which the parent antibody binds. Examples of antigen-binding fragments include, but are not limited to: (i) a Fab fragment, which can be a monovalent fragment composed of a V_{H}-C_{H}1 chain and a V_{L}-C_{L} chain; (ii) a F(ab′)2 fragment which can be a bivalent fragment composed of two Fab fragments linked by a disulfide bridge at the hinge region; (iii) a Fv fragment, composed of the V_{H} and V_{L} domains of an antibody molecule associated together by noncovalent interaction; (iv) a single chain Fv (scFv), which can be a single polypeptide chain composed of a V_{H} domain and a V_{L} domain via a peptide linker; and (v) a (scFv)₂, which can contain two V_{H} domains linked by a peptide linker and two V_{L} domains, which are associated with the two V_{H} domains via disulfide bridges.

As used herein, the term “chimeric antibody” refers to an antibody containing polypeptides from different sources, e.g., different species. In some embodiments, in chimeric antibodies, the variable region of both light and heavy chains may mimic the variable region of antibodies derived from one species of mammal (e.g., a non-human mammal such as mouse, rabbit and rat), while the constant region may be homologous to the sequences in antibodies derived from another mammal such as a human.

As used herein, the term “humanized antibody” refers to an antibody comprising a framework region originated from a human antibody and one or more CDRs from a non-human (usually a mouse or rat) immunoglobulin.

As used herein, the term “human antibody” refers to an antibody in which essentially the entire sequences of the light chain and heavy chain sequences, including the complementary determining regions (CDRs), are from human genes. In some circumstances, the human antibodies may include one or more amino acid residues not encoded by human germline immunoglobulin sequences e.g. by mutations in one or more of the CDRs, or in one or more of the FRs, such as to, for example, decrease possible immunogenicity, increase affinity, and eliminate cysteines that might cause undesirable folding, etc.

As used herein, the term “specific binds” or “specifically binding” refers to a non-random binding reaction between two molecules, such as the binding of the antibody to an epitope of its target antigen. An antibody that “specifically binds” to a target antigen or an epitope is a term well understood in the art, and methods to determine such specific binding are also well known in the art. An antibody “specifically binds” to a target antigen if it binds with greater affinity/avidity, more readily, and/or greater duration than it binds to other substances. In other words, it is also understood by reading this definition that, for example, an antibody that specifically binds to a first target antigen may or may not specifically or preferentially bind to a second target antigen. As such, “specific binding” or “preferential binding” does not necessarily require (although it can include) exclusive binding. Generally, the affinity of the binding can be defined in terms of a dissociation constant (K_{D}). Typically, specifically binding when used with respect to an antibody can refer to an antibody that specifically binds to (recognize) its target with an KD value less than about 10⁻⁷ M, such as about 10⁻⁸ M or less, such as about 10⁻⁹ M or less, about 10⁻¹⁰ M or less, about 10⁻¹¹ M or less, about 10⁻¹² M or less, or even less, and binds to the specific target with an affinity corresponding to a K_{D} that is at least ten-fold lower than its affinity for binding to a non-specific antigen (such as BSA or casein), such as at least 100 fold lower, e.g. at least 1,000 fold lower or at least 10,000 fold lower.

As used herein, the term “nucleic acid” or “polynucleotide” can refer to a polymer composed of nucleotide units. Polynucleotides include naturally occurring nucleic acids, such as deoxyribonucleic acid (“DNA”) and ribonucleic acid (“RNA”) as well as nucleic acid analogs including those which have non-naturally occurring nucleotides. Polynucleotides can be synthesized, for example, using an automated DNA synthesizer. It will be understood that when a nucleotide sequence is represented by a DNA sequence (i.e., A, T, G, C), this also includes an RNA sequence (i.e., A, U, G, C) in which “U” replaces “T.” The term “cDNA” refers to a DNA that is complementary or identical to an mRNA, in either single stranded or double stranded form.

As used herein, the term “complementary” refers to the topological compatibility or matching together of interacting surfaces of two polynucleotides. A first polynucleotide is complementary to a second polynucleotide when the nucleotide sequence of the first polynucleotide is identical to the nucleotide sequence of the polynucleotide binding partner of the second polynucleotide. Thus, the polynucleotide whose sequence 5′-ATATC-3′ is complementary to a polynucleotide whose sequence is 5′-GATAT-3′.”

As used herein, the term “encoding” refers to the natural property of specific sequences of nucleotides in a polynucleotide (e.g., a gene, a cDNA, or an mRNA) to serve as templates for synthesis of other polymers and macromolecules in biological processes having either a given sequence of RNA transcripts (i.e., rRNA, tRNA and mRNA) or a given sequence of amino acids and the biological properties resulting therefrom. Therefore, a gene encodes a protein if transcription and translation of mRNA produced by that gene produces the protein in a cell or other biological system. It is understood by a skilled person that numerous different polynucleotides and nucleic acids can encode the same polypeptide as a result of the degeneracy of the genetic code. It is also understood that skilled persons may, using routine techniques, make nucleotide substitutions that do not affect the polypeptide sequence encoded by the polynucleotides described there to reflect the codon usage of any particular host organism in which the polypeptides are to be expressed. Therefore, unless otherwise specified, a “nucleotide sequence encoding an amino acid sequence” encompasses all nucleotide sequences that are degenerate versions of each other and that encode the same amino acid sequence.

As used herein, the term “recombinant nucleic acid” refers to a polynucleotide or nucleic acid having sequences that are not naturally joined together. A recombinant nucleic acid may be present in the form of a vector. “Vectors” may contain a given nucleotide sequence of interest and a regulatory sequence. Vectors may be used for expressing the given nucleotide sequence (expression vector) or maintaining the given nucleotide sequence for replicating it, manipulating it or transferring it between different locations (e.g., between different organisms). Vectors can be introduced into a suitable host cell for the above-described purposes. A “recombinant cell” refers to a host cell that has had introduced into it a recombinant nucleic acid. “A transformed cell” mean a cell into which has been introduced, by means of recombinant DNA techniques, a DNA molecule encoding a protein of interest.

Vectors may be of various types, including plasmids, cosmids, episomes, fosmids, artificial chromosomes, phages, viral vectors, etc. Typically, in vectors, the given nucleotide sequence is operatively linked to the regulatory sequence such that when the vectors are introduced into a host cell, the given nucleotide sequence can be expressed in the host cell under the control of the regulatory sequence. The regulatory sequence may comprise, for example and without limitation, a promoter sequence (e.g., the cytomegalovirus (CMV) promoter, simian virus 40 (SV40) early promoter, T7 promoter, and alcohol oxidase gene (AOX1) promoter), a start codon, a replication origin, enhancers, a secretion signal sequence (e.g., α-mating factor signal), a stop codon, and other control sequence (e.g., Shine-Dalgamo sequences and termination sequences). Preferably, vectors may further contain a marker sequence (e.g., an antibiotic resistant marker sequence) for the subsequent screening/selection procedure. For purpose of protein production, in vectors, the given nucleotide sequence of interest may be connected to another nucleotide sequence other than the above-mentioned regulatory sequence such that a fused polypeptide is produced and beneficial to the subsequent purification procedure. Said fused polypeptide includes a tag for purpose of purification e.g. a His-tag.

As used herein, the term “treatment” refers to the application or administration of one or more active agents to a subject afflicted with a disorder, a symptom or condition of the disorder, or a progression of the disorder, with the purpose to cure, heal, alleviate, relieve, alter, remedy, ameliorate, improve, or affect the disorder, the symptom or condition of the disorder, the disabilities induced by the disorder, or the progression or predisposition of the disorder.

The present invention is based, at least in part, on the development of combined cancer therapy using an EpCAM inhibitor and a Wnt signaling inhibitor.

EpCAM is known as a CSC marker in many cancer types, as EpEX contributes to a tumorigenic microenvironment and EpICD is a well-studied promoter of cell motility, proliferation, survival and metastasis (Gires et al., 2009; Lin et al., 2012; Park et al., 2016; Yu et al., 2017; Liang et al., 2018; Herreros-Pomares et al, 2018; Gires et al., 2020; Chen et al., 2020). More importantly, soluble EpICD is known to form a multi-protein nuclear complex with the β-Catenin and a scaffolding protein named Four and one-half LIM domains protein 2 (FHL2). This protein complex translocates to the nucleus, where it associates with T-Cell Factor (TCF) or Lymphoid Enhancer Factor 1 (LEF-1) and DNA, in a manner reminiscent of the canonical Wnt signaling pathway (Maetzel et al., 2009; Ralhan et al., 2010; Park et al., 2016; Yu et al., 2017). However, it is unknown whether EpEX is somehow coordinated with the Wnt pathway. Thus, we sought to determine whether EpEX is functionally involved in Wnt signaling, with the hope that we may be able to target EpEX in order to modulate intracellular signaling of EpICD and β-Catenin in CSCs.

In the present invention, it is surprisingly found that EpEX interacts with Wnt receptors, FZD6/7 and LRP5/6, promoting nuclear translocation of β-Catenin; and EpICD promotes transcription of Wnt receptors and sternness factors. It is also found that Wnt ligands and EpEX activate EpCAM cleaving enzymes TACE and γ-secretase as positive feedback, augmenting production of EpEX and EpICD. These mechanisms induce cancer sternness, and exploiting an EpCAM inhibitor targeting EpEX (such as an anti-EpCAM neutralizing antibody e.g. EpAb2-6) and a Wnt inhibitor (e.g. a porcupine inhibitor, e.g. LGK974) is found to induce apoptosis of CSCs. This combination provides a potential therapeutic strategy, particularly giving superior effects in reducing tumor progression and/or metastasis, and/or prolonging survival of a cancer patient.

As used herein, “combined therapy” refers to treatment that combines two or more therapeutic agents or approaches. “Combination” means that two or more therapeutic agents or approaches are given to the same subject, at the same time or in sequence. Preferably, combined therapy provides synergistic effects.

As used herein, the term “synergistic effect” may mean and include a cooperative action resulted in a combination of two or more active agents in which the combined activity of the two or more active agents exceeds the sum of the activity of each active agent alone. The term “synergistic effect” may also refer to that two or more active agents when used together provide combined activity such that a lower dose of each may be used to achieve comparative or enhanced activity when single agent is used.

Therefore, the present invention provides a combined therapy for treating cancer, comprising administering to a subject in need thereof a combination comprising (i) an effective amount of a first inhibitory agent that inhibits the activation of EpCAM signaling (an EpCAM inhibitor); and (ii) an effective amount of a second inhibitory agent that inhibits the activation of Wnt signaling (a Wnt inhibitor).

In some embodiments, the first inhibitory agent (an EpCAM inhibitor) reduces production (or release) of EpEX and/or blocks binding of EpEX to a Wnt receptor In some instances, the first inhibitory agent is an antibody directed to EpEX or an antigen-binding fragment thereof.

In some embodiments, an anti-EpEX antibody as used herein specifically binds to the EGF-like domain I of EpCAM (aa 27-59 of EpCAM) and the EGF-like domain II of EpCAM (aa 66-135 of EpCAM). Specifically, an anti-EpEX antibody as used herein has a specific binding affinity to an epitope within the sequence of CVCENYKLAVN (aa 27 to 37) (SEQ ID NO: 20) located in the EGF-like domain I, and KPEGALQNNDGLYDPDCD (aa 83 to 100) (SEQ ID NO: 19) located in the EGF-like domain II More specifically, an anti-EpEX antibody as used herein recognizes the NYK motif (aa 31-33) within domain I and the LYD motif (aa 94-96) within domain II in EpCAM. In contrast, a number of other antibodies (e.g. MT201, M97, 323/A3 and edrecolomab) target only the well-described EGF I domain of EpCAM. The distinct features of the anti-EpEX antibody according to the present invention from other antibodies are described below.

| | |
|---|---|
| anti-EpEX antibody according | binding to both domain I and domain |
| to the present invention | II and effective in inducing |
| | apoptosis of cancer cells |
| Other antibodies | binding to domain I only and failing |
| (e.g. MT201, M97, | to induce apoptosis of cancer cells |
| 323/A3 and edrecolomab) | |

One certain anti EpEX antibody as used herein is EpAb2-6 as shown in Examples below. The amino acid sequences of the heavy chain variable region (V_{H}) and light chain variable region (V_{L}), and their complementary determining regions (HC CDR1, HC CDR2 and HC CDR3) (LC CDR1, LC CDR2 and LC CDR3) of EpAb2-6 are as shown in Table 1 below. The anti-EpEX antibody of the present invention includes EpAb2-6 and its functional variant.

TABLE 1
V_{H} domain

| FW1 | CDR1 | FW2 | CDR2 |
|---|---|---|---|
| VKLQESGPELKKPGETVK | GYTFTDYSMH | WVKQAPGKGLKWMG | INTETGEP |
| ISCKAS | (SEQ ID NO: 2) | W | (SEQ ID NO: 4) |
| (SEQ ID NO: 1) | | (SEQ ID NO: 3) | |
| FW3 | CDR3 | FW4 | |
| TYADDFKGRFAFSLETSA | TAVY | WGQGTTVTVSS | |
| STAYLQINNLKNEDTATY | (SEQ ID NO: 6) | (SEQ ID NO: 7) | |
| FCAR | | | |
| (SEQ ID NO: 5) | | | |

V_{L} domain

| FW1 | CDR1 | FW2 | CDR2 |
|---|---|---|---|
| DIQMTQSPSSLSASLGERV | RASQEISVSLS | WLQQEPDGTIKRLIY | ATSTLDS |
| SLTC | (SEQ ID NO: 9) | (SEQ ID NO: 10) | (SEQ ID NO: 11) |
| (SEQ ID NO: 8) | | | |
| FW3 | CDR3 | FW4 | |
| GVPKRFSGSRSGSDYSLTI | LQYASYPWT | FGGGTKLEIKRADAAP | |
| SSLESEDFVDYYC (SEQ ID | (SEQ ID NO: 13) | TVS | |
| NO: 12) | | (SEQ ID NO: 14) | |

Full-length amino acid sequences of heavy chain and light chain

| | |
|---|---|
| heavy chain | VKLQESGPELKKPGETVKISCKASGYTFTDYSMHWVKQAPGKGLKWMGWINTETGEPTYAD |
| | DFKGRFAFSLETSASTAYLQINNLKNEDTATYFCARTAVYWGQGTTVTVSS (SEQ ID |
| | NO: 15) |
| light chain | DIQMTQSPSSLSASLGERVSLTCRASQEISVSLSWLQQEPDGTIKRLIYATSTLDSGVPKR |
| | FSGSRSGSDYSLTISSLESEDFVDYYCLQYASYPWTFGGGTKLEIKRADAAPTVS (SEQ |
| | ID NO: 16) |

In some embodiments, the anti-EpEX antibody of the present invention is a functional variant of EpAb2-6 which is characterized in comprising (a) a VH comprising HC CDR1 of SEQ ID NO: 2, HC CDR2 of SEQ ID NO: 4, and HC CDR3 of SEQ ID NO: 6; and (b) a VL comprising LC CDR1 of SEQ ID NO: 9, LC CDR2 of SEQ ID NO: 11, and HC CDR3 of SEQ ID NO: 13, or an antigen-binding fragment thereof.

In some embodiments, the anti-EpEX antibody of the present invention, having (a) a VH comprising HC CDR1 of SEQ ID NO: 2, HC CDR2 of SEQ ID NO: 4, and HC CDR3 of SEQ ID NO: 6; and (b) a VL comprising LC CDR1 of SEQ ID NO: 9, LC CDR2 of SEQ ID NO: 11, and HC CDR3 of SEQ ID NO: 13, can comprise a V_{H} comprising SEQ ID NO: 15 or an amino acid sequence substantially identical thereto and a V_{L} comprising SEQ ID NO: 16 or an amino acid sequence substantially identical thereto. Specifically, the anti-EpEX antibody of the present invention includes a V_{H} comprising an amino acid sequence has at least 80% (e.g. 82%, 84%, 85%, 86%, 88%, 90%, 92%, 94%, 95%, 96%, 98%, or 99%) identity to SEQ ID NO:15, and a V_{L} comprising an amino acid sequence has at least 80% (e.g. 82%, 84%, 85%, 86%, 88%, 90%, 92%, 94%, 95%, 96%, 98%, or 99%) identity to SEQ ID NO:16. The anti-EpEX antibody of the present invention also includes any recombinantly (engineered)-derived antibody encoded by the polynucleotide sequence encoding the relevant V_{H} or V_{L} amino acid sequences as described herein.

The term “substantially identical” can mean that the relevant amino acid sequences (e.g., in FRs, CDRs, V_{H}, or V_{L}) of a variant differ insubstantially as compared with a reference antibody such that the variant has substantially similar binding activities (e.g., affinity, specificity, or both) and bioactivities relative to the reference antibody. Such a variant may include minor amino acid changes. It is understandable that a polypeptide may have a limited number of changes or modifications that may be made within a certain portion of the polypeptide irrelevant to its activity or function and still result in a variant with an acceptable level of equivalent or similar biological activity or function. In some examples, the amino acid residue changes are conservative amino acid substitution, which refers to the amino acid residue of a similar chemical structure to another amino acid residue and the polypeptide function, activity or other biological effect on the properties smaller or substantially no effect. Typically, relatively more substitutions can be made in FR regions, in contrast to CDR regions, as long as they do not adversely impact the binding function and bioactivities of the antibody (such as reducing the binding affinity by more than 50% as compared to the original antibody). In some embodiments, the sequence identity can be about 80%, 82%, 84%, 85%, 86%, 88%, 90%, 92%, 94%, 95%, 96%, 98%, or 99%, or higher, between the reference antibody and the variant. Variants can be prepared according to methods for altering polypeptide sequence known to one of ordinary skills in the art such as those found in references which compile such methods, e.g. Molecular Cloning: A Laboratory Manual, J. Sambrook, et al., eds., Second Edition, Cold Spring Harbor Laboratory Press, Cold Spring Harbor, New York, 1989. For example, conservative substitutions of amino acids include substitutions made amongst amino acids within the following groups: (i) A, G; (ii) S, T; (iii) Q, N; (iv) E, D; (v) M, I, L, V; (vi) F, Y, W; and (vii) K, R, H.

The antibodies described herein may be animal antibodies (e.g., mouse-derived antibodies), chimeric antibodies (e.g., mouse-human chimeric antibodies), humanized antibodies, or human antibodies. The antibodies described herein may also include their antigen-binding fragments e.g. a Fab fragment, a F(ab′)2 fragment, a Fv fragment, a single chain Fv (scFv) and a (scFv)₂. The antibodies or their antigen-binding fragments can be prepared by methods known in the art

More details of an anti-EpEX antibody as used herein are as described in U.S. Pat. No. 9,187,558, the relevant disclosures of each of which are incorporated by reference herein for the purposes or subject matter referenced herein.

Numerous methods conventional in this art are available for obtaining antibodies or antigen-binding fragments thereof.

In some embodiments, the antibodies provided herein may be made by the conventional hybridoma technology. In general, a target antigen e.g. a tumor antigen optionally coupled to a carrier protein, e.g. keyhole limpet hemocyanin (KLH), and/or mixed with an adjuvant, e.g complete Freund's adjuvant, may be used to immunize a host animal for generating antibodies binding to that antigen. Lymphocytes secreting monoclonal antibodies are harvested and fused with myeloma cells to produce hybridoma. Hybridoma clones formed in this manner are then screened to identify and select those that secrete the desired monoclonal antibodies.

In some embodiments, the antibodies provided herein may be prepared via recombinant technology. In related aspects, isolated nucleic acids that encode the disclosed amino acid sequences, together with vectors comprising such nucleic acids and host cells transformed or transfected with the nucleic acids, are also provided.

For examples, nucleic acids comprising nucleotide sequences encoding the heavy and light chain variable regions of such an antibody can be cloned into expression vectors (e.g., a bacterial vector such as an E. coli vector, a yeast vector, a viral vector, or a mammalian vector) via routine technology, and any of the vectors can be introduced into suitable cells (e.g., bacterial cells, yeast cells, plant cells, or mammalian cells) for expression of the antibodies. Examples of nucleotide sequences encoding the heavy and light chain variable regions of the antibodies as described herein are as shown in Table 1. Examples of mammalian host cell lines are human embryonic kidney line (293 cells), baby hamster kidney cells (BHK cells), Chinese hamster ovary cells (CHO cells), African green monkey kidney cells (VERO cells), and human liver cells (Hep G2 cells). The recombinant vectors for expression the antibodies described herein typically contain a nucleic acid encoding the antibody amino acid sequences operably linked to a promoter, either constitutive or inducible. Typical vectors contain transcription and translation terminators, initiation sequences, and promoters useful for regulation of the expression of the nucleic acid encoding the antibody. The vectors optionally contain selection markers for both prokaryotic and eukaryotic systems. In some examples, both the heavy and light chain coding sequences are included in the same expression vector. In other examples, each of the heavy and light chains of the antibody is cloned into an individual vector and produced separately, which can be then incubated under suitable conditions for antibody assembly.

The recombinant vectors for expression the antibodies described herein typically contain a nucleic acid encoding the antibody amino acid sequences operably linked to a promoter, either constitutive or inducible. The recombinant antibodies can be produced in prokaryotic or eukaryotic expression systems, such as bacteria, yeast, insect and mammalian cells. Typical vectors contain transcription and translation terminators, initiation sequences, and promoters useful for regulation of the expression of the nucleic acid encoding the antibody. The vectors optionally contain selection markers for both prokaryotic and eukaryotic systems. The antibody protein as produced can be further isolated or purified to obtain preparations that substantially homogeneous for further assays and applications. Suitable purification procedures, for example, may include fractionation on immunoaffinity or ion-exchange columns, ethanol precipitation, sodium dodecyl sulfate polyacrylamide gel electrophoresis (SDS-PAGE), high-performance liquid chromatography (HPLC), ammonium sulfate precipitation, and gel filtration.

When a full-length antibody is desired, coding sequences of any of the V_{H} and V_{L} chains described herein can be linked to the coding sequences of the Fc region of an immunoglobulin and the resultant gene encoding a full-length antibody heavy and light chains can be expressed and assembled in a suitable host cell, e.g., a plant cell, a mammalian cell, a yeast cell, or an insect cell.

Antigen-binding fragments can be prepared via routine methods. For example, F(ab′)₂ fragments can be generated by pepsin digestion of an full-length antibody molecule, and Fab fragments that can be made by reducing the disulfide bridges of F(ab′)₂ fragments. Alternatively, such fragments can also be prepared via recombinant technology by expressing the heavy and light chain fragments in suitable host cells and have them assembled to form the desired antigen-binding fragments either in vivo or in vitro. A single-chain antibody can be prepared via recombinant technology by linking a nucleotide sequence coding for a heavy chain variable region and a nucleotide sequence coding for alight chain variable region. Preferably, a flexible linker is incorporated between the two variable regions.

One antibody can be further modified to conjugate one or more additional elements at the N- and/or C-terminus of the antibody such as another protein and/or a drug or carrier. Preferably, an antibody conjugated with an additional element retains the desired binding specificity and therapeutic effect while providing additional properties resulted from the additional element that aids, for example, in solubility, storage or other handling properties, cell permeability, half-life, reduction in hypersensitivity, controls delivery and/or distribution. Other embodiments include the conjugation of a label e.g. a dye or fluorophore for assays, detection, tracking and the like. In some embodiments, an antibody can be conjugated to an additional element such as a peptide, dye, fluorophore, carbohydrates, anti-cancer agent, lipid, etc. In addition, an antibody can be attached to the surface of a liposome directly via an Fc region, for example, to form immunoliposomes.

In some embodiments, the second inhibitory agent (a Wnt inhibitor) blocks binding of a Wnt ligand to a Wnt receptor protein. Specifically, the Wnt ligand is not EpEX.

In some embodiments, the second inhibitory agent (a Wnt inhibitor) is a porcupine inhibitor. Porcupine (PORCN) is a membrane bound O-acyltransferase that mediates palmitoylation of Wnt family proteins which is required for secretion and biologic activity of Wnt. Thus, a porcupine inhibitor can inhibit Wnt signaling. Small-molecule PORCN inhibitory compounds include, for example, LGK-974, ETC-159, and Wnt-C59. Table 2 shows some examples of small-molecule PORCN inhibitory compounds.

TABLE 2

| | |
|---|---|
| 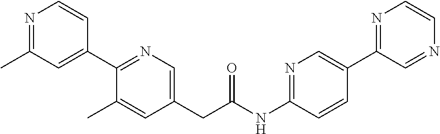 [SMILES: Cc1cc(-c2ncc(CC(=O)Nc3ccc(-c4cnccn4)cn3)cc2C)ccn1] | LGK-974 2-(2′,3-dimethyl-[2,4′-bipyridin]-5- yl)-N-(5-(pyrazin-2-yl)pyridin-2- yl)acetamide |
| 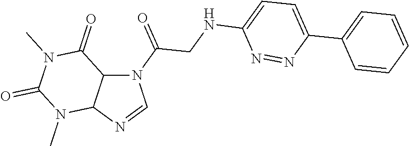 [SMILES: CN1C(=O)C2C(N=CN2C(=O)CNc2ccc(-c3ccccc3)nn2)N(C)C1=O] | ETC-159 1,3-dimethyl-7-((6- phenylpyridazin-3-yl)glycyl)- 3,4,5,7-tetrahydro-1H-purine-2,6- dione |
| 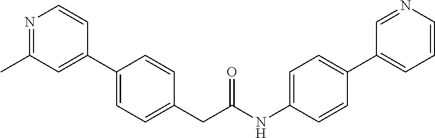 [SMILES: Cc1cc(-c2ccc(CC(=O)Nc3ccc(-c4cccnc4)cc3)cc2)ccn1] | Wnt-C59 2-(4-(2-methylpyridin-4- yl)phenyl)-N-(4-(pyridin-3- yl)phenyl)acetamide |

As used herein the term “small-molecule porcupine (PORCN) inhibitory compound” or “small-molecule PORCN inhibitor” may include a small-molecule compound that inhibits or binds to porcupine. Unless indicated otherwise, all references herein to small-molecule PORCN inhibitors include references to pharmaceutically acceptable salts, solvates, hydrates and complexes thereof, and to solvates, hydrates and complexes of pharmaceutically acceptable salts thereof, including polymorphs, stereoisomers, and isotopically labeled versions thereof.

As used herein, the term “pharmaceutically acceptable salt” includes acid addition salts. “Pharmaceutically acceptable acid addition salts” refer to those salts which retain the biological effectiveness and properties of the free bases, which are formed with inorganic acids such as hydrochloric acid, hydrobromic acid, sulfuric acid, nitric acid, phosphoric acid and the like, and organic acids such as acetic acid, propionic acid, pyruvic acid, maleic acid, malonic acid, succinic acid, fumaric acid, tartaric acid, citric acid, benzoic acid, mandelic acid, methanesulfonic acid, ethanesulfonic acid, p-toluenesulfonic acid, salicylic acid, trifluoroacetic acid and the like. The term “pharmaceutically acceptable salt” also includes base salts. Suitable base salts are formed from bases which form non-toxic salts. Examples include the aluminum, arginine, benzathine, calcium, choline, diethylamine, diolamine, glycine, lysine, magnesium, meglumine, olamine, potassium, sodium, tromethamine and zinc salts.

The term “effective amount” used herein refers to the amount of an active ingredient to confer a desired biological effect in a treated subject or cell. The effective amount may change depending on various reasons, such as administration route and frequency, body weight and species of the individual receiving said pharmaceutical, and purpose of administration. Persons skilled in the art may determine the dosage in each case based on the disclosure herein, established methods, and their own experience.

A subject to be treated by the method of treatment as described herein can be a mammal, more preferably a human. Mammals include, but are not limited to, farm animals, sport animals, pets, primates, horses, dogs, cats, mice and rats.

As used herein, “pharmaceutically acceptable carrier” means that the carrier is compatible with an active ingredient in the composition, and preferably can stabilize said active ingredient and is safe to the receiving individual. Said carrier may be a diluent, vehicle, excipient, or matrix to the active ingredient. Typically, a composition comprising an EpCAM inhibitor, a Wnt inhibitor or a combination thereof can be formulated in a form of a solution such as an aqueous solution e.g. a saline solution or it can be provided in powder form. Appropriate excipients also include lactose, sucrose, dextrose, sorbose, mannose, starch, Arabic gum, calcium phosphate, alginates, tragacanth gum, gelatin, calcium silicate, microcrystalline cellulose, polyvinyl pyrrolidone, cellulose, sterilized water, syrup, and methylcellulose. The composition may further contain pharmaceutically acceptable auxiliary substances as required to approximate physiological conditions, for example, pH adjusting and buffering agents, such as sodium acetate, sodium chloride, potassium chloride, calcium chloride, sodium lactate and the like. The form of the composition may be tablets, pills, powder, lozenges, packets, troches, elixers, suspensions, lotions, solutions, syrups, soft and hard gelatin capsules, suppositories, sterilized injection fluid, and packaged powder. The composition of the present invention may be delivered via any physiologically acceptable route, such as oral, parenteral (such as intramuscular, intravenous, subcutaneous, and intraperitoneal), transdermal, suppository, and intranasal methods. In certain embodiments, the composition of the present invention is administered as a liquid injectable formulation which can be provided as a ready-to-use dosage form or as a reconstitutable stable powder.

In some embodiments, the two active components used in the present invention, an EpCAM inhibitor and a Wnt inhibitor, may be formulated as a mixture or independently, in kit form, for simultaneous, separate or sequential administration to a subject. Each component may be formulated together with a suitable pharmaceutically acceptable carrier for proper administration routes. In some embodiments, an EpCAM inhibitor and a Wnt inhibitor may be provided in suitable packaging units where an EpCAM inhibitor or a composition comprising the same and a Wnt inhibitor or a composition comprising the same are present within distinct packaging units.

According to the present invention, combined use of an EpCAM inhibitor and a Wnt inhibitor provides synergistic effects in treating cancer, particularly in inducing apoptosis of cancer cells, reducing or suppressing tumor progression, cancer sternness and/or metastasis, and/or prolonging survival of a cancer patient, as compared with the EpCAM inhibitor or the Wnt inhibitor alone. In particular, as shown in the examples (e.g. Example 2.7), in the metastatic model, treatment with either an EpCAM neutralizing antibody (EpAb2-6) as an EpCAM inhibitor or a combination of an EpCAM neutralizing antibody (EpAb2-6) as an EpCAM inhibitor plus an EpCAM inhibitor (LGK974) can prolong animal survival while most of the animals in the control IgG or EpCAM inhibitor (LGK974)-treated groups exhibit distinct metastases and decreased overall survival; similarly, in the orthotopic model, animals in control IgG or EpCAM inhibitor (LGK974)-treated groups develop significant tumor and display low median survival while the EpCAM neutralizing antibody (EpAb2-6) treated group exhibits slower tumor progression and higher median survival, and surprisingly the combination treatment using the EpCAM neutralizing antibody (EpAb2-6) and an EpCAM inhibitor (LGK974) provides synergistic pronounced effect in reducing tumor progression (about 60% (4/6) animals are found completely free of tumors) and the overall survival are prolonged.

In some embodiments, an EpCAM inhibitor and a Wnt inhibitor are administered simultaneously, separately or sequentially to provide a synergistic anticancer or anti-metastasis effect and in particular the cancer is sensitive to the synergistic combination.

In some embodiments, the cancer is selected from the group consisting of lung cancer, brain cancer, breast cancer, cervical cancer, colon cancer, gastric cancer, head and neck cancer, kidney cancer, leukemia, liver cancer, ovarian cancer, pancreatic cancer, prostate cancer, skin cancer and testicular cancer

The present invention is further illustrated by the following examples, which are provided for the purpose of demonstration rather than limitation. Those of skill in the art should, in light of the present disclosure, appreciate that many changes can be made in the specific embodiments which are disclosed and still obtain a like or similar result without departing from the spirit and scope of the invention.

### EXAMPLES

Epithelial cell adhesion molecule (EpCAM), a pleiotropic type-1 transmembrane glycoprotein, is a known cancer stem cell marker, yet the mechanisms underlying its involvements in cancer sternness remain elusive. Here, we used a colorectal cancer (CRC) model system to uncover and define interactions between EpCAM and Wnt signaling that promote cancer sternness. We demonstrate that the extracellular domain of EpCAM (EpEX) functions as a ligand for Wnt receptor proteins, frizzled6/7 and LRP5/6, inducing signal transduction. Further, the intracellular domain (EpICD) upregulates transcription of genes encoding such Wnt receptors and critical sternness factors. Interestingly, EpEX-induced Wnt signaling activates TACE and γ-secretase enzymes that augment shedding of EpEX and EpICD, establishing a positive feedback loop. In the line of this mechanism, our EpCAM neutralizing antibody (EpAb2-6) and a porcupine inhibitor (LGK974) can each partially attenuate cancer sternness properties, while their combination abolishes the phenomena and induces apoptosis in CRC cells. The combined treatment also markedly thwarts tumor progression in metastatic and orthotopic animal models of human CRC, substantially prolonging animal survival. We conclude that EpCAM activation stimulates Wnt signaling to promote cancer stemness. Thus, the combination of EpAb2-6 and porcupine inhibitors might effectively suppress cancer stemness, overcome drug resistance and improve CRC treatment.

### 1. Material and Methods

### 1.1 Cell Culture

Experiments were performed using HCT116, HT29, CT26, SW620, HEK293T and HeLa cell lines. HCT116, HT29, HEK293T were cultured in Dulbecco's Modified Eagle Medium (DMEM) (Gibco), while CT26 cells and SW620 cells were cultured in RPMI1640 (Gibco) and L-15 (Gibco) medium respectively. Media were supplemented with 10% Fetal Bovine Serum (FBS, Gibco), 1% L-glutamine (Gibco), and 1% penicillin and streptomycin (P/S) (Gibco). All cells except SW620 were grown in 5% C02 at 37° C. SW620 cells were grown in 0% C02 at 37° C.

For the growth curve, 10⁴ cells were seeded in triplets using six-well plates for each cell line. Each triplet was counted using hemocytometer, and the counts were averaged each day, from day 1 to 8. After collection of the entire dataset, points were plotted to analyze the growth curve and calculate cell doubling time.

### 1.2 Cells Fractionation

Cells (1×10⁶) were seeded overnight and further allowed to grow in serum free conditions. Then cells were further treated with 20 μg/mL mEpAb2-6 or hEpAb2-6 or MT201 for 6 h or with 400 ng/mL LGK974 (MedChemExpress) for 9 h or combination as indicated. Samples were fractioned into cytosol and nuclear extracts using nuclear/cytosol fractionation kit (Biovision) according to the manufacturer's protocol. The fractions were then subjected to western blot analysis.

### 1.3 Western Blotting

For western blotting, cells were extracted using radioimmunoprecipitation assay (RIPA) buffer [(0.01 M sodium phosphate, pH 7.2), 150 mM NaCl, 2 mM EDTA, 50 mM NaF, 1% Nonidet P-40, 1% sodium deoxycholate, and 0.1% SDS)] containing phosphatase inhibitor (Roche) and protease inhibitor (Roche) cocktail. Equal amounts of protein were separated by SDS-PAGE and then transferred to PVDF membranes. The membranes were blocked with 3% BSA in TBST (Blocking solution) and incubated with necessary primary antibodies in the blocking solution overnight at 4° C. Membranes were then incubated with HRP-conjugated secondary antibodies in the blocking solution for 1 h at room temperature, and protein expression were detected. Antibodies used: anti-α-tubulin (Sigma), anti-EpCAM (abeam), anti-Active β-Catenin (Millipore), anti-total β-Catenin (abeam), anti-Frizzled 6 (CST), anti-Frizzled 7 (Santa Cruz Biotech), anti-LRP5 (abcam), anti-Phospho-LRP5 (abcam), anti-Phospho-LRP6 (CST), anti-LRP6 (CST) and anti-EpEX antibody EpAb3-5 (Produced in house), anti-ADAM17 (abcam), anti-phopho-ADAM17 (abcam), anti-Presenilin2 (abcam), anti-phospho-Presenilin2 (S327) (abcam), anti-phospho-Presenilin2 (S330) (abcam) and anti-Axin2 (CST).

### 1.4 TCF Activity

Cells were plated at 5×10³ cells per well and grown overnight in 12-well plates. Then, cells were transiently transfected with TOP-Flash TCF reporter plasmid (Millipore) using poly-jet transfection reagent (SignaGen). At 48 h post transfection, cells were treated with 20 g/mL Anti-EpCAM EpAb2-6 (Produced in house) or MT201 (Produced in house) for 6 h or 400 ng/mL LGK974 (MedChemExpress) for 9 h or the combination, as indicated. In addition, cells were treated with EpEX (Produced in house by Expi293 Expression System) or recombinant Wnt3A (R & D systems) or the combination for 8 h. Finally, cells were lysed, and the luciferase assay was performed.

### 1.5 Immunohistochemical Staining

Human colon cancer tissue arrays were purchased from Biomax. Sections were de-waxed in xylene and rehydrated through a series of solutions with declining alcohol concentrations. Antigen retrieval was performed concomitantly in the Trilogy TM (Cell Marque). For peroxidase blocking, sections were incubated with methanol containing H₂O₂ (3%) for 20 min at room temperature (RT). Sections were further washed with PBS and incubated with 1% bovine serum albumin (BSA) in PBS for 30 min at RT to block non-specific binding. Following primary antibody, anti-active $3-Catenin (Millipore) and anti-EpEX antibody EpAb3-5 (Produced in house) were applied, and samples were incubated at 4° C. overnight. Next, sections were washed with PBS containing 0.1% Tween 20 (PBST0.1) (Thermo) and treated with the Super Sensitive Super Enhancer reagent for 20 min at RT. Then, samples were rinsed three times with PBST0.1. Sections were subsequently treated with Polymer-HRP reagent for 30 min at RT and then were rinsed three times with PBST0.1. Next, 3,3′-Diaminobenzidine (DAB) was used as a chromogen to visualize peroxidase activity. The quantification of protein intensities was performed using Fiji-Image J software.

### 1.6 Immunofluorescence Staining

Glass slides were used in 24-well plates and coated with 0.1% gelatin. Further, 3×10⁴ cells were seeded in serum free medium overnight. Cells were treated with 20 μg/mL EpAb2-6 for 6 h, or 400 ng/mL LGK974 (MedChemExpress) for 9 h or the combination. Cells were washed using ice-cold PBS and fixed with 4% paraformaldehyde for 15 min at RT and washed with ice-cold PBS. Further, cells were permeabilized using 0.1% triton-X in PBS for 20 min and subsequently washed with PBS. Cells were blocked with 3% BSA in PBS for 1 h at RT. Next, cells were treated with primary antibody anti-Active β-Catenin (Millipore) overnight. Then, cells were washed and treated with secondary antibody in 3% BSA containing PBS along with DAPI for 1 h at RT. Samples were then washed in PBS five times and mounted for microscopy. Nuclear β-Catenin intensities were calculated using IMARIS (Oxford Instruments) software.

1.7 Quantitative Real Time PCR (qPCR)

Total RNA was extracted using TRI reagent and 5 μg of the total RNA was further reverse-transcribed using oligo (dT) primer with reverse transcriptase. Quantitative real time RT-PCR (qPCR) was performed on cDNA using the Light Cycler 480 SYBR Green-I Master kit and the LightCycler480 System. The gene expression levels of each sample were normalized to the expression levels of glyceraldehyde 3-phosphate dehydrogenase (GAPDH) or β-actin. The primers used in qPCR are listed in Table 3.

TABLE 3

| Gene | Forward, Reverse |
|---|---|
| Human EpCAM | GCCAGTGTACTTCAGTTGGTGC (SEQ ID NO: |
| | 21) |
| | CCCTTCAGGTTTTGCTCTTCTCC (SEQ ID NO: |
| | 22) |
| Human FZD6 | ATTTTGGTGTCCAAGGCATC (SEQ ID NO: 23) |
| | TATTGCAGGCTGTGCTATCG (SEQ ID NO: 24) |
| Human FZD7 | GTGCAGTGTTCTCCCGAACT (SEQ ID NO: 25) |
| | GAACGGTAAAGAGCGTCGAG (SEQ ID NO: 26) |
| Human LRP5 | ACCGGAACCACGTCACAG (SEQ ID NO: 27) |
| | GGGTGGATAGGGGTCTGAGT (SEQ ID NO: 28) |
| Human LRP6 | AGGCACTTACTTCCCTGCAA (SEQ ID NO: 29) |
| | GGGCACAGGTTCTGAATCAT (SEQ ID NO: 30) |
| Human AXIN2 | TGACTCTCCTTCCAGATCCCA (SEQ ID NO: 31) |
| | TGCCCACACTAGGCTGACA (SEQ ID NO: 32) |
| Human GAPDH | AGGTCGGAGTCAACGGATTT (SEQ ID NO: 33) |
| | TAGTTGAGGTCAATGAAGGG (SEQ ID NO: 34) |
| Human OCT4 | ACATGTGTAAGCTGCGGCC (SEQ ID NO: 35) |
| | GTTGTGCATAGTCGCTGCTTG (SEQ ID NO: 36) |
| Human SOX2 | TATTTGAATCAGTCTGCCGAG (SEQ ID NO: 37) |
| | ATGTACCTGTTATAAGGATGATATTAGT (SEQ ID |
| | NO: 38) |
| Human c-MYC | AAACACAAACTTGAACAGCTAC (SEQ ID NO: |
| | 39) |
| | ATTTGAGGCAGTTTACATTATGG (SEQ ID NO: |
| | 40) |

### 1.8 Luciferase Reporter Assay

HEK293T packaging cells were co-transfected with packaging plasmid (pCMV-ΔR8.91), envelope (pMDG) and shRNA (shEpCAM #1 and shEpCAM #2) containing plasmid using a Poly JET transfection kit. At 48 h post-transfection, virus-containing supernatants were collected, mixed with fresh medium containing polybrene (8 μg/mL) and incubated with target cells for another 48 h. The transduced cells were selected with necessary antibiotics, and single clones were selected to expand into stable clones.

For EpCAM-Knockout using CRISPR/Cas9, EPCAM CRISPR Guide RNA (target sequence: GTGCACCAACTGAAGTACAC (SEQ ID NO: 41), Vector: pLentiCRISPR v2) was purchased from GenScript; the aforementioned procedure was followed for lentivirus production and clone selection.

### 1.9 Tumorsphere Assay

Cells were seeded in ultra-low adherent 6-well plates (5×10⁴ cells per well) or 24-well plates (1×10³ cells per well) and maintained in DMEM/F-12 supplemented with B27. Further, cells were treated with 20 μg/mL mEpAb2-6, hEpAb2-6 or MT201 (Produced in house), or 400 ng/mL LGK974 (MedChemExpress) or the combination, by direct addition to the culture medium. The entire culture medium, including the treatment component, was changed in every other day. Cells were cultured for 10 days, and on 10^{th} day, spheres were counted and photographed under the microscope.

### 1.10 Colony Formation Assay

Cells were seeded in 12-well plate (5×10³ cells per well) and treated with 20 g/mL mEpAb2-6, hEpAb2-6 or MT201 (Produced in house) or 400 ng/mL LGK974 (MedChemExpress) or combination as indicated in which case the elements were directly added to the culture medium. The medium along with the treatment component were changed in every alternating day and the cells were allowed to grow for 10 days. On the tenth day, cells were washed and fixed with 4% paraformaldehyde and further stained with 1% crystal violet for 30 min. Colonies were washed 3 times with PBS, and images were taken. Furthermore, to measure colony density, the wells were incubated with 0.5% SDS shaking for 2 h at RT. The supernatants were collected, and the absorbance of the solution was taken at 570 nm using a microplate reader.

### 1.11 In Vitro Regeneration Assay

Both control and EpCAM knock out cells (5×10′ cells per well) were subjected to tumor sphere assay in 12-well plates following the protocol as described earlier. 7 days post seeding, plates were imaged and spheres were counted. Further, the spheres were trypsinized into single cells and passed through a cell strainer (BD Falcon) in order to avoid cell clumps, counted and subjected to tumor sphere assay (5×10³ cells per well) and allowed to grow for 7 more days. This procedure was repeated three times. After last regeneration, plates were imaged and spheres were counted.

### 1.12 Interactions Between EpEX and Wnt Receptors

Cells were seeded overnight and harvested with 10 mM EDTA in PBS before being incubated with 2 mM DTSSP (Thermo) cross-linker in order to stabilize the interaction between EpEX and Wnt receptor proteins. Then, Tris (pH 7.5) was added to a final concentration of 20 mM to stop the cross-linking reaction. Cells were then lysed using NP40 buffer (1% by volume NP-40, 150 mM NaCl, 50 mM Tris, pH 8.0) added with protease inhibitor cocktail. Protein-G dyna beads were used to pull down the EpEX-Wnt-receptor complex, and co-immunoprecipitation with western blotting was performed.

### 1.13 Co-Immunoprecipitation (Co-IP) and Subsequent Western Blotting

Co-immunoprecipitation (Co-IP) was performed using Pierce magnetic protein-G dyna beads (Thermo), according to the manufacturer's instructions. Briefly, cells were lysed using NP40 buffer added with protease cocktail inhibitor. The cell lysates containing 500 μg to 1 mg protein were incubated overnight at 4° C. with an antibody for immunoprecipitation. Then, the products were incubated with protein-G dyna beads at 4° C. for 4 h. The beads were pulled down using a magnet and washed three times, and then, sample buffer was added to the protein conjugated beads and cooked for 10 min at 100° C. The final product was subjected to Western blot analysis as described earlier. Antibodies used for pull down and western blot analysis included: Frizzled 6 (CST), Frizzled 7 (Santa Cruz Biotech), LRP5 (abcam), LRP6 (CST) and EpEX (EpAb3-5) (Produced in house).

### 1.14 Enzyme-Linked Immunosorbent Assay (ELISA)

For ELISA, wells (at least 6 wells per single protein) were coated with recombinant FZD6 (Proteintech), recombinant FZD7 (Proteintech), recombinant LRP5 (Proteintech), recombinant LRP6 (Proteintech) overnight at 4° C. The wells were then blocked with 1% BSA and treated with EpEX-his (Produced in house by Expi293 Expression System) for 2 hours. Otherwise, EpEX-His was incubated with EpAb2-6 overnight and the complex was used to treat the protein-coated plates for 2 hours. Further anti-His antibody (abeam) was used and TMB was performed to record optical density at 450 nm.

### 1.15 Apoptosis Assay

Cells were seeded in 24-well plate (5×10 cells per well) overnight and then treated with 20 μg/mL mEpAb2-6, hEpAb2-6 or MT201, or 2 g/mL LGK974 (MedChemExpress) or the combination for 24 hrs. Cell pellets were collected, and an apoptosis assay was performed using an Annexin-V/PI apoptosis kit (BD Biosciences). Results were read by flow cytometry analysis percentage apoptotic cells was calculated.

### 1.16 Luciferase Reporter Assay

The cells were seeded in 24-well plates (1×10⁴ cells/well) and incubated at 37° C. for 24 h. The culture media were refreshed, and the cells were transfected with respective reporter plasmids (TCF reporter or Wnt receptor promoter reporter) by PolyJET (SignaGen). The transfected efficiency was normalized by co-transfected with pRL-TK (20 ng) as an internal control. Additional treatments were conducted as indicated. Firefly luciferase and Renilla luminescence were measured 48 h post-transfection using the Dual-Glo Luciferase Assay System (Promega), according to the manufacturer's recommendations.

### 1.17 Tumorigenic Potential In Vivo

NSG mice were divided into two groups with equal numbers. EpCAM-control or EpCAM-knockout HCT116 cells were subcutaneously transplanted (10³ cells) in the right flank of each animal (n=6 in each group). Tumors were allowed to grow, and tumor dimensions were measured twice a week using slide calipers. Once the tumor volume reached 2000 mm³ (As defined by IACUC, Academia Sinica) for any mouse in the experiment, all the animals were sacrificed and tumor weights and volumes were measured. None of the data was excluded.

### 1.18 TACE Activity Assay

Cells were seeded overnight in 24-well plate (1×10 cells per well) and further treated with 250 ng/mL EpEX-His or 100 ng/mL Wnt3A (R&D Systems) for 8 h. Then, TACE activity was measured using the InnoZyme TACE activity kit (Merck). In brief, cell lysates were prepared with RIPA buffer and loaded into a TACE antibody-coated plate and incubated at RT for 1 h with slight shaking. Further, the lysates were removed and the plate was washed three times. Substrate was added into each well and incubated for 5 h at 37° C. Finally, the fluorescence signal of the reaction product was detected at excitation of 324 nm and emission of 405 nm using a microplate reader.

### 1.19 γ-Secretase Activity

The γ-secretase activity was measured using the protocol described by Liao et al (2004) (Liao et al., 2004). In brief, cells were transiently transfected with the control plasmid and tetracycline-inducible γ-secretase plasmid harboring luciferase (Liao et al., 2004) (the plasmids were generous gifts from Dr. Yung-Feng Liao, ICOB, Academia Sinica). Cells were treated with 250 ng/mL EpEX-His or 100 ng/mL Wnt3A (R&D Systems) for 8 h. Further, cells were lysed using passive lysis buffer and subjected to the luciferase assay.

### 1.20 Wnt Receptor Promoter Reporter Plasmid Construction

The putative promoter regions of LRP5 (−1187 to +200), LRP6 (−1543 to +55), FZD6 (−1385 to +205) and FZD7 (−1285 to +116) were cloned from HeLa genomic DNA and fused to pGL4.18 plasmid (Promega, USA). The genomic DNA was extracted using a Genomic DNA Isolation Kit (NovelGene, TW) according to the manufacturer's recommendations. The primers used to generate the PCR fragments of Wnt receptor promoters are listed in Table 4.

TABLE 4

| Assay | Gene | Primer |
|---|---|---|
| Cloning | LRP5 | F: GCC GGT ACC AAG AAG GGT GGA ACC |
| | PM | GTG TC (SEQ ID NO: 42) |
| | | R: GCC AAG CTT TGT GGA GGG GGA TAG |
| | | GGA CTT (SEQ ID NO: 43) |
| | LRP6 | F: GCC GGT ACC CAG AGA CCT GGA TTG |
| | PM | GGC TG (SEQ ID NO: 44) |
| | | R: GCC CTC GAG TCA GGA GCA CAC AGA |
| | | AGC TG (SEQ ID NO: 45) |
| | FZD6 | F: CTC AGC TAG CAC CAC TGT CCC CTA |
| | PM | (SEQ ID NO: 46) |
| | | R: AAC ACC CTC GAG GGT GAA CGG GCT |
| | | (SEQ ID NO: 47) |
| | FZD7 | F: GCC GGT ACC CTA ACG CGA CTC CTG |
| | PM | GTC AC (SEQ ID NO: 48) |
| | | R: GCC AAG CTT TTC TCT CCG TGG TAC |
| | | GGC T (SEQ ID NO: 49) |

PM: Promoter

### 1.21 GSK3 and CK1 Activity on ADAM17 and Presenilin2 Phosphorylation

To study the kinase activity of GSK3 and CK1, 10⁶ cells were seeded overnight and treated with GSK3 inhibitor BIO (Sigma) or CK1 inhibitor PF-670462 (selleckchem) for 8 h. Cells were then lysed with RIPA buffer and subjected to western blot analysis to study phosphorylation of ADAM17 and Presenilin2. Otherwise, cells were treated with EpEX (Produced in house) or recombinant Wnt3A (R & D systems) or the combination for 8 h to study phosphorylation of ADAM17 and Presenilin2.

### 1.22 Plasmid Transfection and Protein (EpICD) Delivery

All plasmid transfections procedures were performed as indicated using Polyjet DNA transfection reagent (SignaGen Lab). The protocol was performed according to the manufacturer's instructions. EpICD protein (produced in house by Expi293 expression system) delivery was performed using Pierce Protein Transfection Reagent Kit (Thermo Scientific). The protocol was performed as directed by the kit.

### 1.23 Tumor Transplantation and Therapeutic Studies in Mice

All animal experiments were approved and performed according to the regulations from the IACUC at Academia Sinica. HCT116 cells (1×10⁶) were injected via tail vein for the metastatic model. Otherwise, 2×10⁵ HCT116 cells with luciferase were surgically transplanted into the cecum wall for the orthotopic model. Male NOD/SCID mice, approximately 6 to 8 weeks old, were used for animal experiments (n=5 and n=6 for each treatment group in metastatic and orthotopic models respectively). At 72-h post-injection/transplantation, mice were randomly distributed into four different treatment groups. For the treatments, animals were injected with 20 mg/kg IgG or EpAb2-6 via tail vein twice a week for four weeks, or fed by oral gavage with either vehicle [0.5% methylcellulose (Sigma-Aldrich) and 0.5% Tween80 (Sigma-Aldrich)] or 5 mg/kg LGK974 (MedChemExpress) formulated with the vehicle on alternating days for four weeks, or animals were treated with the combination of both inhibitor and antibody. For the metastatic model, survival was the main endpoint. For the orthotopic model, tumor progression was monitor using bioluminescence imaging. To image the tumors, intraperitoneal injection of D-Luciferin (GOLD BIO) was performed, and images were taken 10 min post-injection.

### 1.24 Statistical Analysis

Statistical analysis was performed using GraphPad Prism (GraphPad Software). Data were analyzed using one-way ANOVA or two-way ANOVA as necessary and stated in the figure legends followed by Bonferroni multiple correction. The P values below 0.05 was considered significant and the stars assigned to each significant value are indicated in figure legends. The error bars in all included data set represent ±SD of the mean. All experiments performed at least three times. None of the data in the study were excluded.

### 2. Results

2.1 EpCAM Expression is Associated with β-Catenin Activity

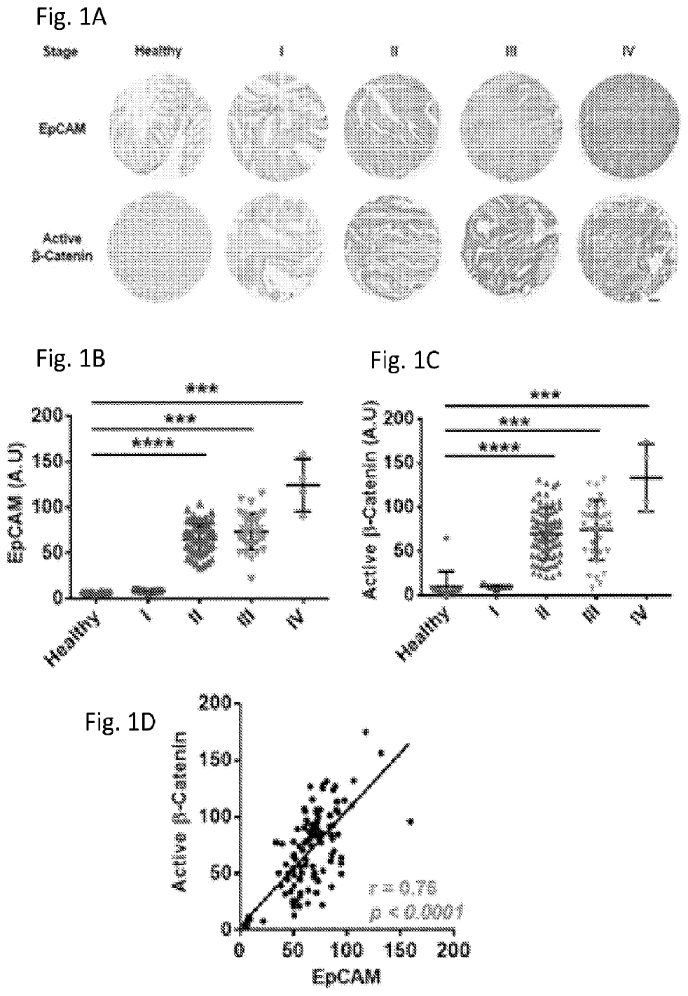

To begin the study, we asked whether EpCAM expression is correlated with active β-Catenin in CRC tissue samples. We performed immunohistochemistry (IHC) on 120 patient tissue samples, finding that the levels of EpCAM and β-Catenin were elevated in disease samples compared to healthy tissue samples. In addition, the levels of both proteins were also found to increase with increased grade of CRC (FIG. 1A, FIG. 1C). In fact, correlation analysis showed that the expression of EpCAM is strongly co-related with that of the active β-Catenin (Pearson's correlation co-efficient r=0.76, p<0.0001) (FIG. 1D). Thus, we next sought to explore whether and how EpCAM participates in canonical Wnt signaling.

### 2.2 EpEX is Involved in Nuclear Translocation of β-Catenin

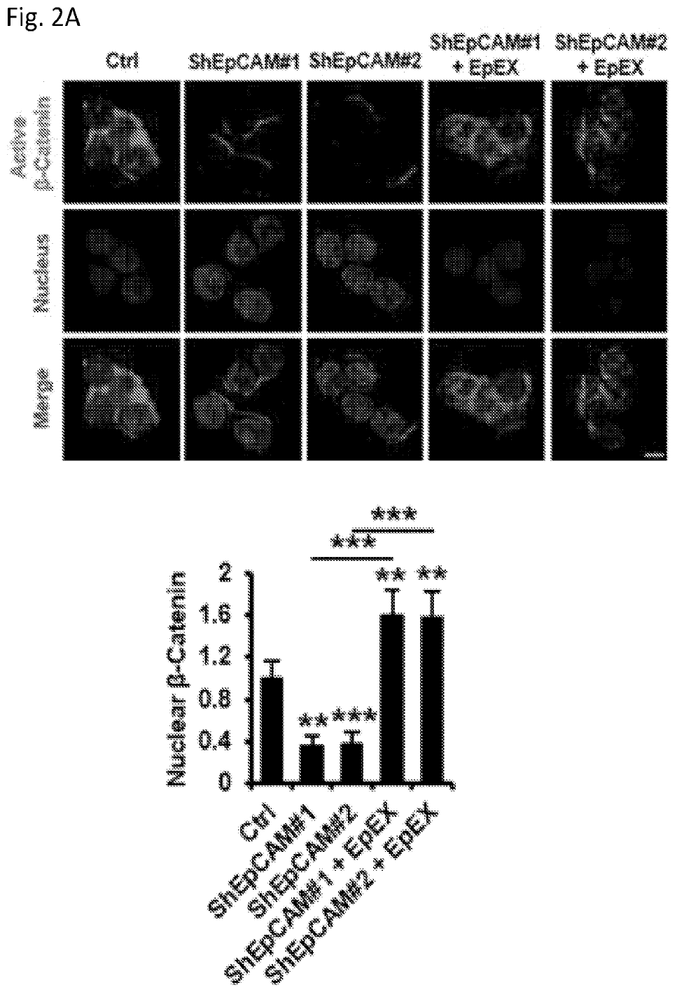

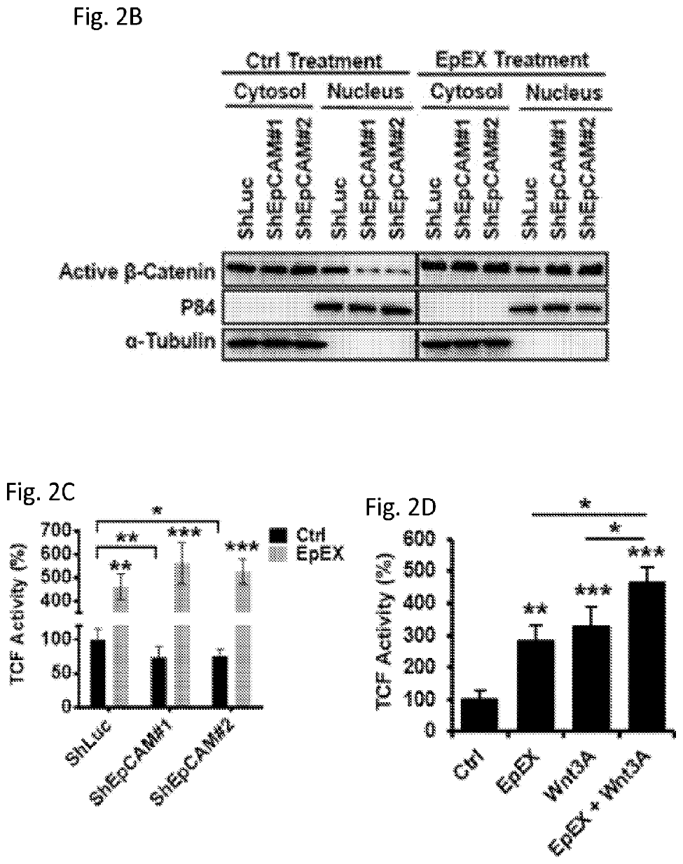

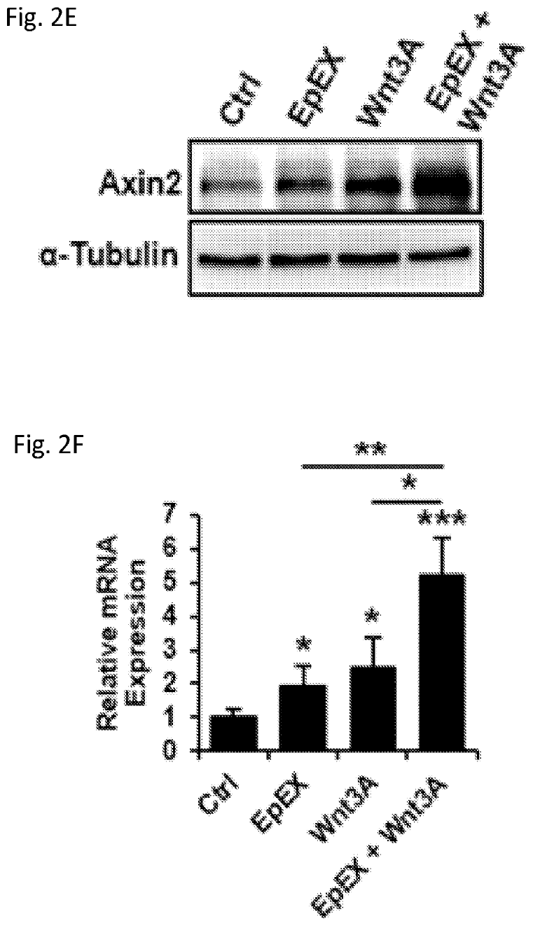

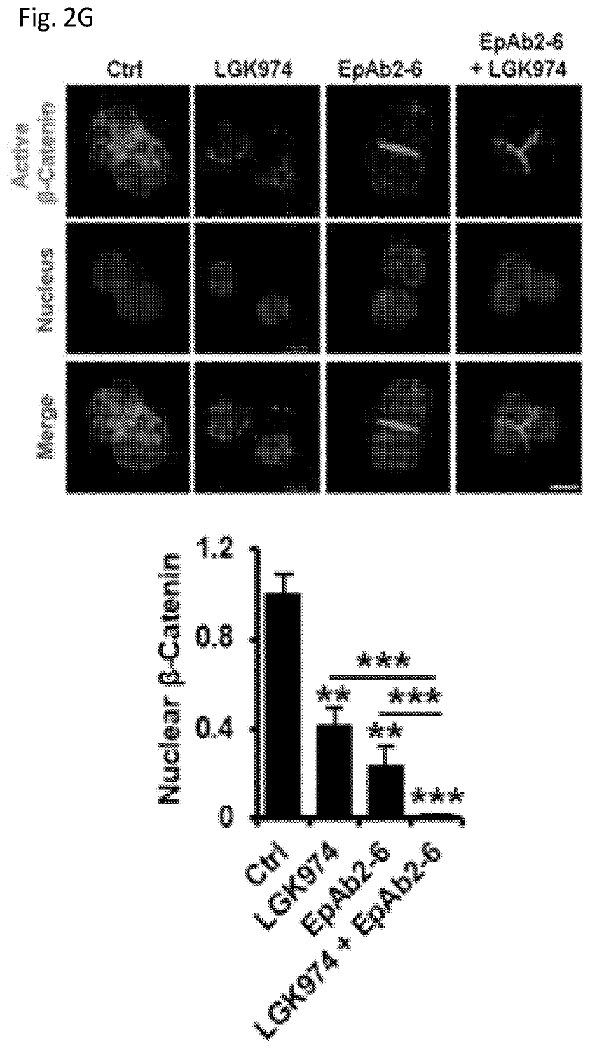

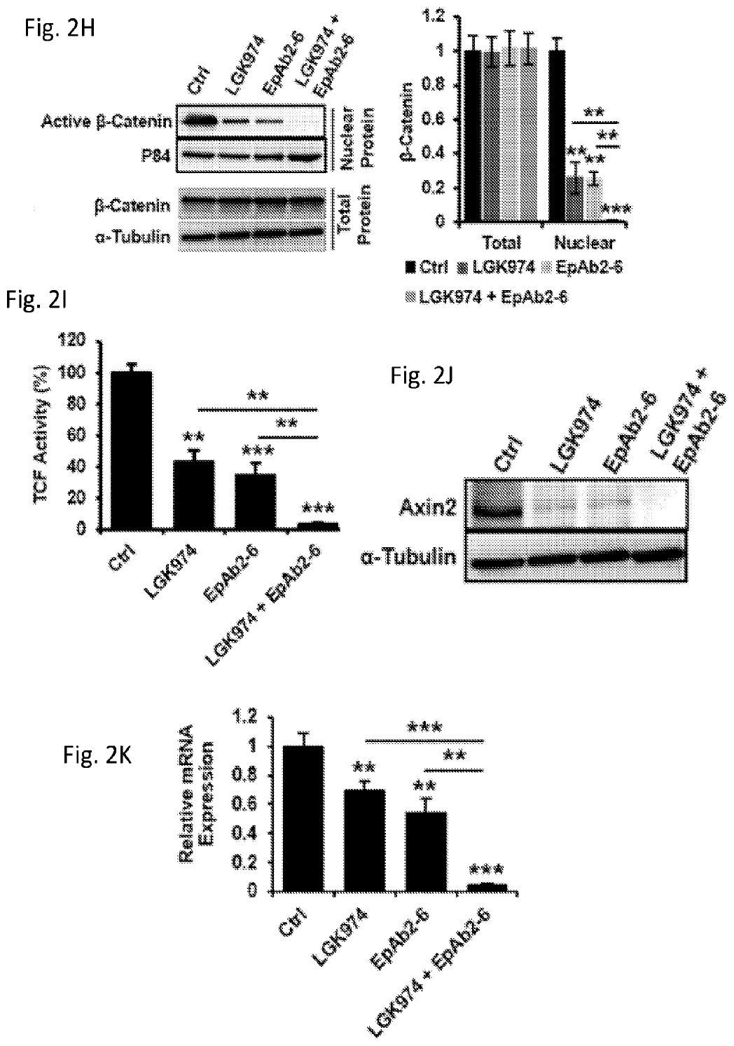

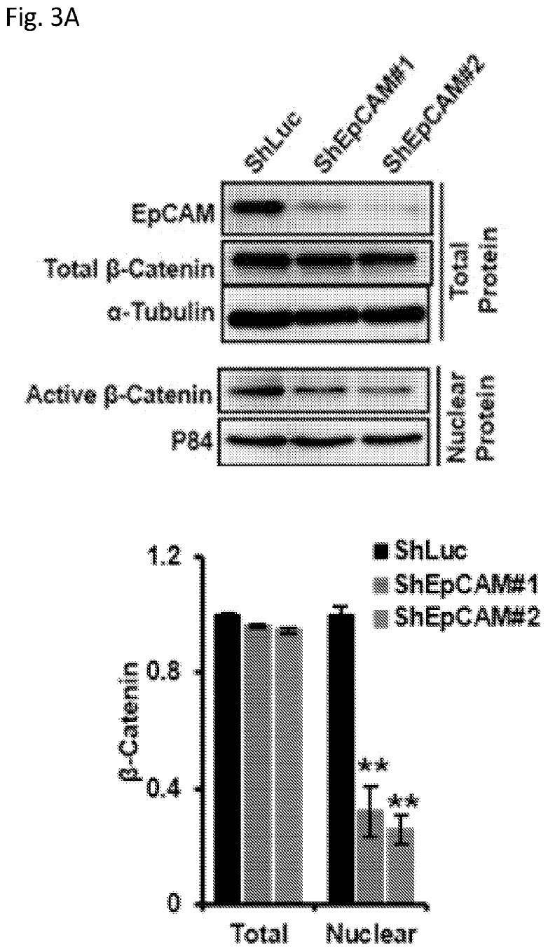

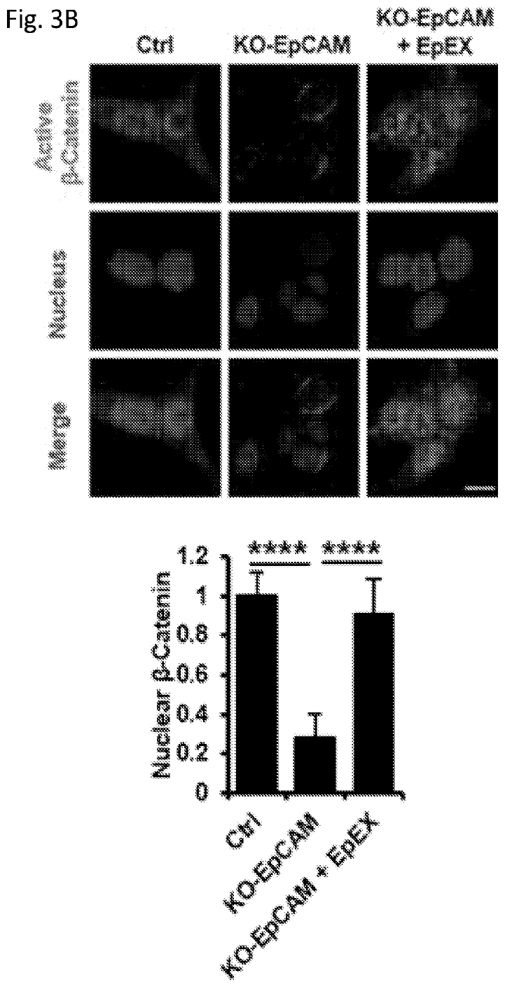

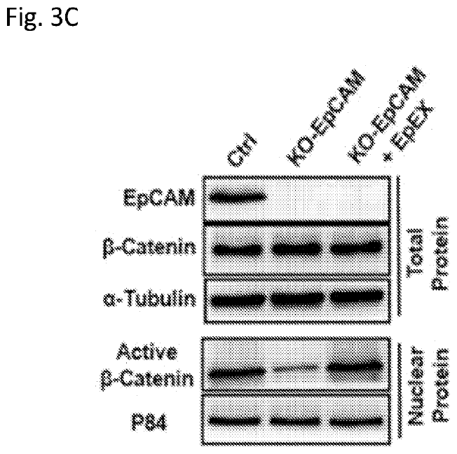

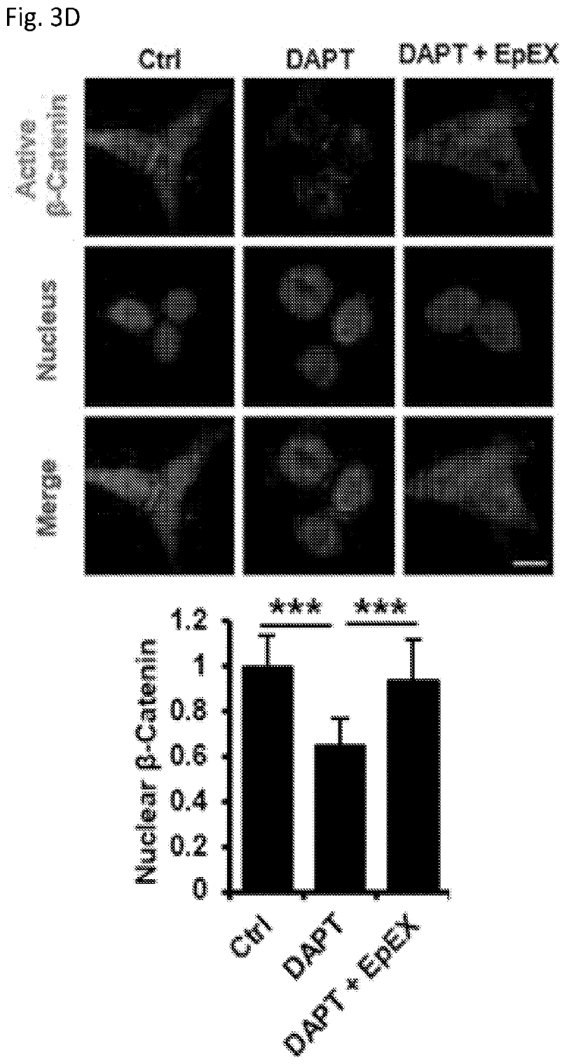

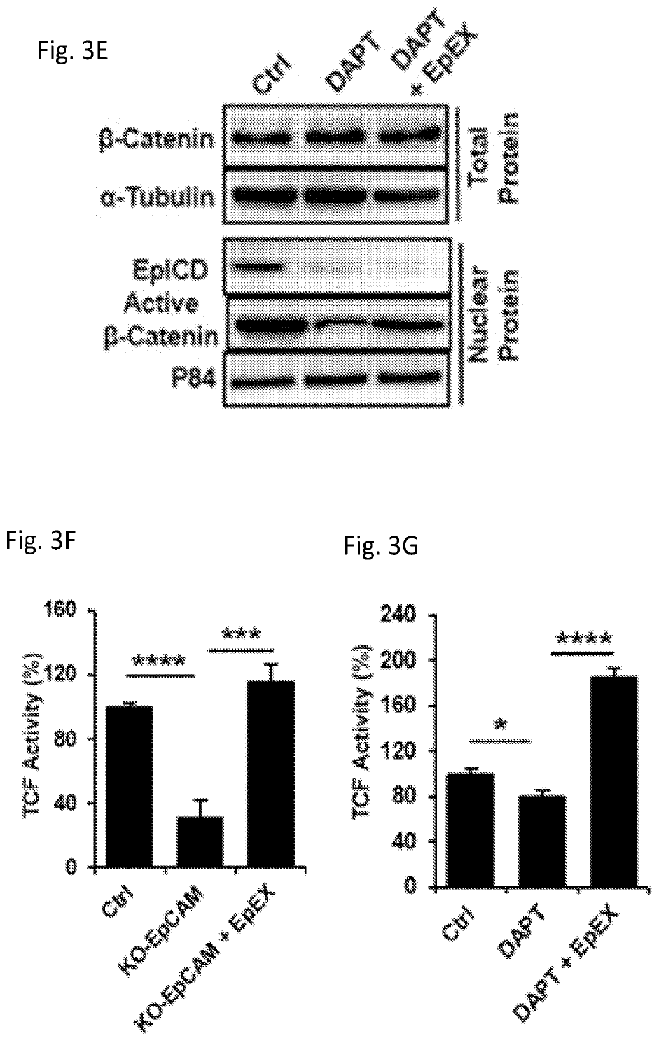

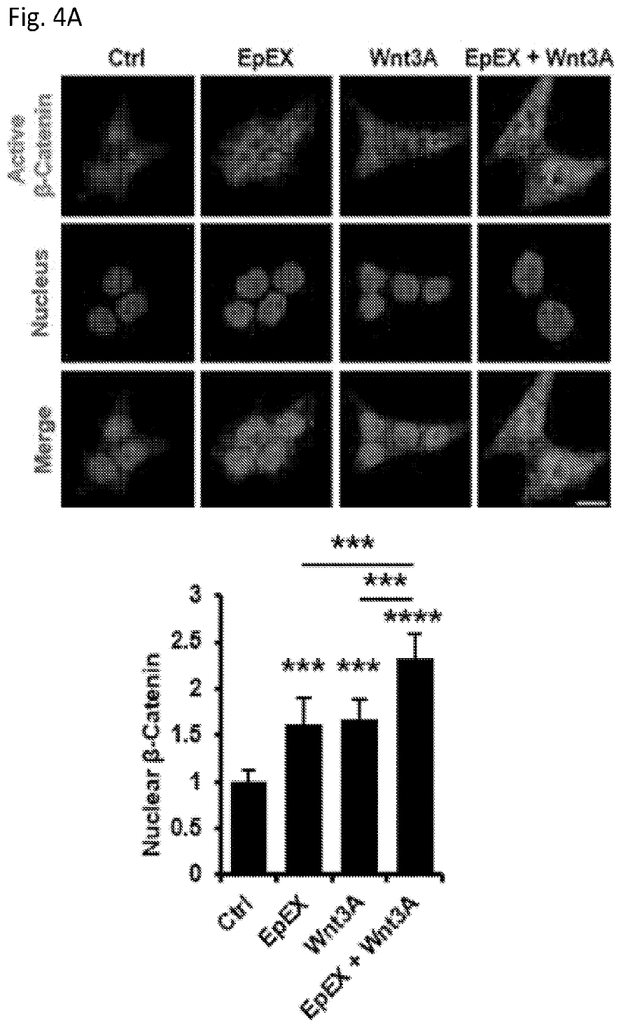

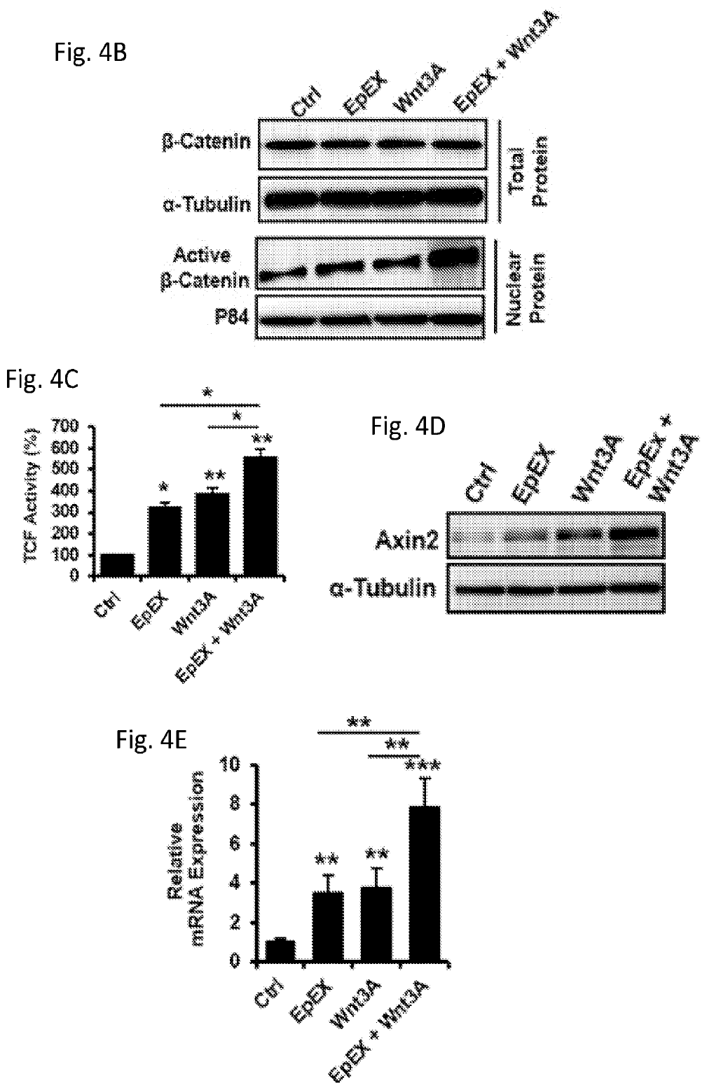

We next tested whether EpCAM promotes nuclear localization of 3-Catenin, which is the standard readout for canonical Wnt signaling. EpCAM-knockdown (shEpCAM) or EpCAM-knockout (KO-EpCAM) colon cancer cells were immunostained for active β-Catenin. We found that knockdown or knockout EpCAM significantly decreased β-Catenin nuclear accumulation (FIG. 2A, FIG. 2B, FIG. 3A, FIG. 3B, FIG. 3C). Notably, the complex of EpICD and β-Catenin, along with binding partner FHL2, is known to translocate to the nucleus and regulate transcription of EpCAM target genes with the help of transcription factors such as TCF or LEF (Lin et al., 2012; Maetzel et al., 2009; Park et al., 2016). However, β-Catenin without EpICD might still translocate to nucleus and bind to such factors to transcribe Wnt target genes (Maetzel et al., 2009; Nusse and Clevers, 2017). Therefore, to investigate if EpEX could regulate nuclear translocation of the protein independent of EpICD, shEpCAM or KO-EpCAM cells were treated with exogenous EpEX. This treatment stimulated a significant increase in nuclear accumulation of β-Catenin (FIG. 2A, FIG. 2B, FIG. 3A, FIG. 3B, FIG. 3C). In addition, treating wild-type cells with DAPT, a γ-secretase inhibitor, decreased nuclear translocation of β-Catenin, but treating the cells with both EpEX and DAPT rescued nuclear accumulation of the protein (FIG. 3D, FIG. 3E). Furthermore, we monitored TCF activity with a luciferase reporter in EpEX-treated EpCAM-knockdown and knockout cells (FIG. 2C and FIG. 3F). Similar to IFS and western results, EpCAM-knockdown or knockout cells exhibited decreased TCF activity compared to control cells, and treating the cells with EpEX significantly rescued the phenomenon. In addition, DAPT treatment of wild-type cells marginally decreased TCF activity, but the phenomena were significantly increased with combined treatment of EpEX and DAPT (FIG. 3G). Taken altogether, these observations suggest a potential role for EpEX in stimulating nuclear translocation of β-Catenin independent of EpICD. Next, we investigated the individual and combined effects of EpEX and Wnt proteins (We used recombinant Wnt3A) on nuclear translocation of β-Catenin and TCF activity in wild-type cells (FIG. 2D, FIG. 4A, FIG. 4B, FIG. 4C). We noticed that either EpEX or Wnt3A could increase nuclear translocation of β-Catenin and TCF activity, while the combination of both further increased the signal. We further wanted to test whether the treatments also regulate direct target genes of Wnt pathway, such as Axin2 (FIG. 2E, FIG. 2F, FIG. 4D, FIG. 4E). Indeed, we noticed that similar to the TCF activity results, either EpEX or Wnt3A increased Axin2 expression, while the combination treatment enhanced such activity. Together, these results suggested that EpEX might activate the Wnt pathway, while EpICD further participates in downstream signaling.

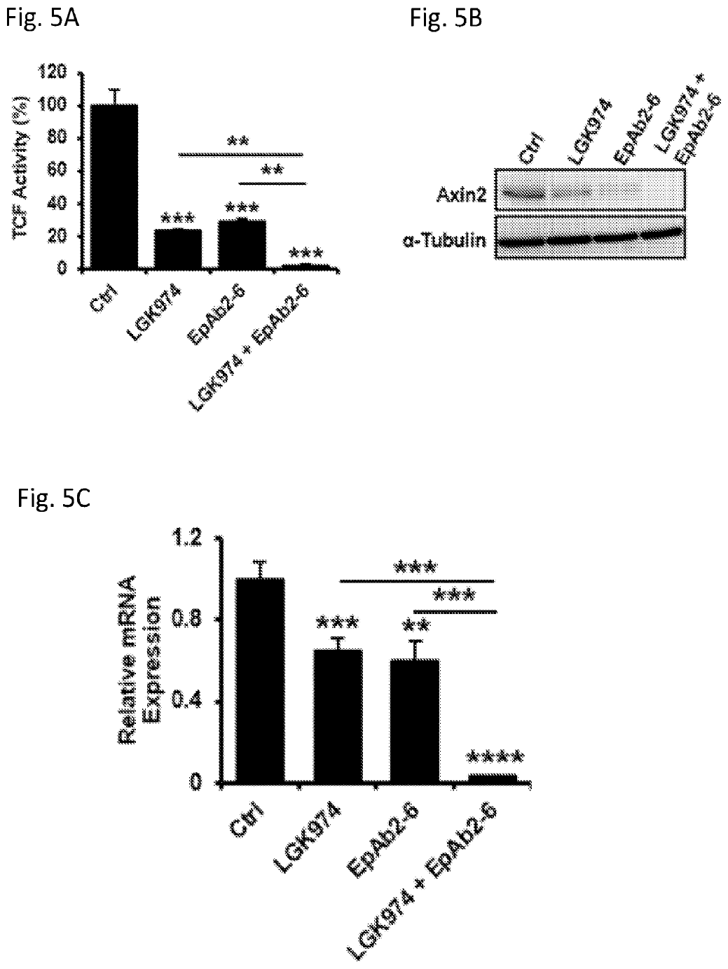

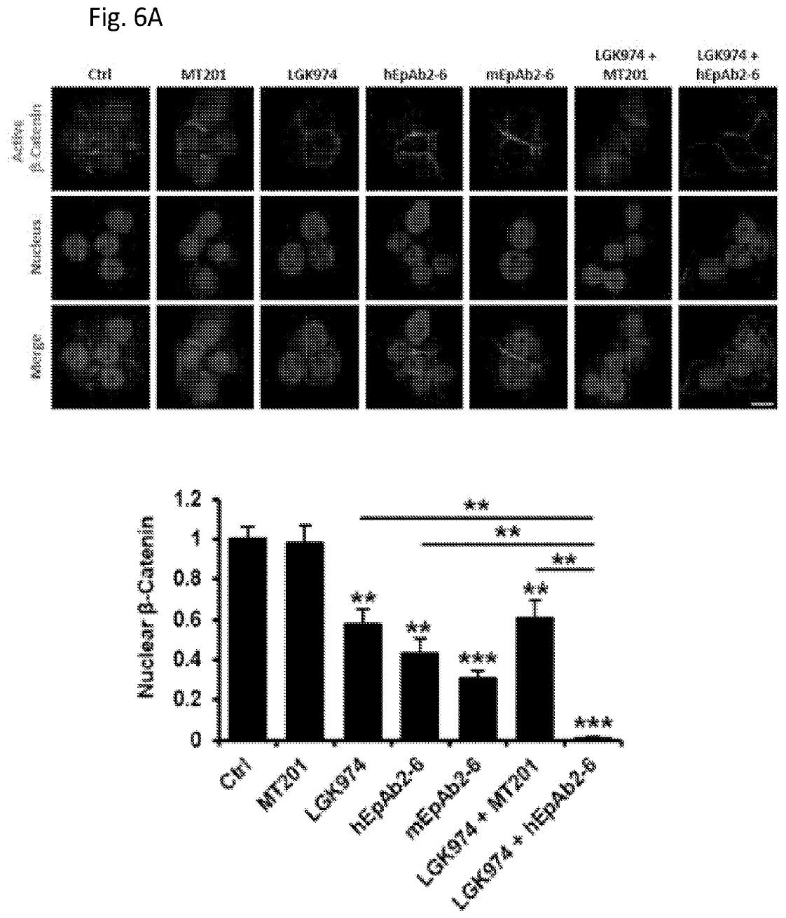

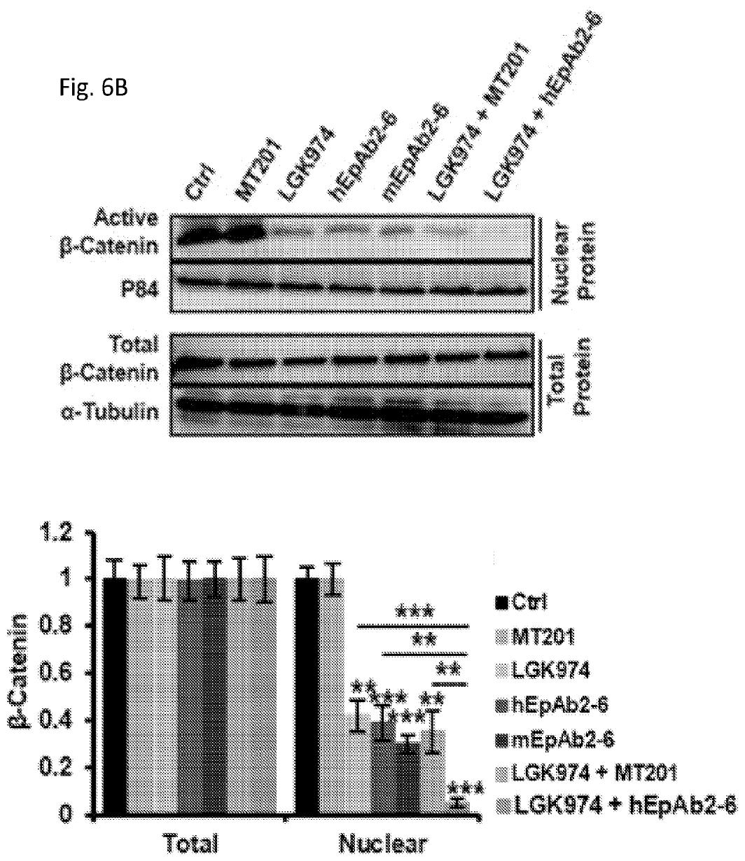

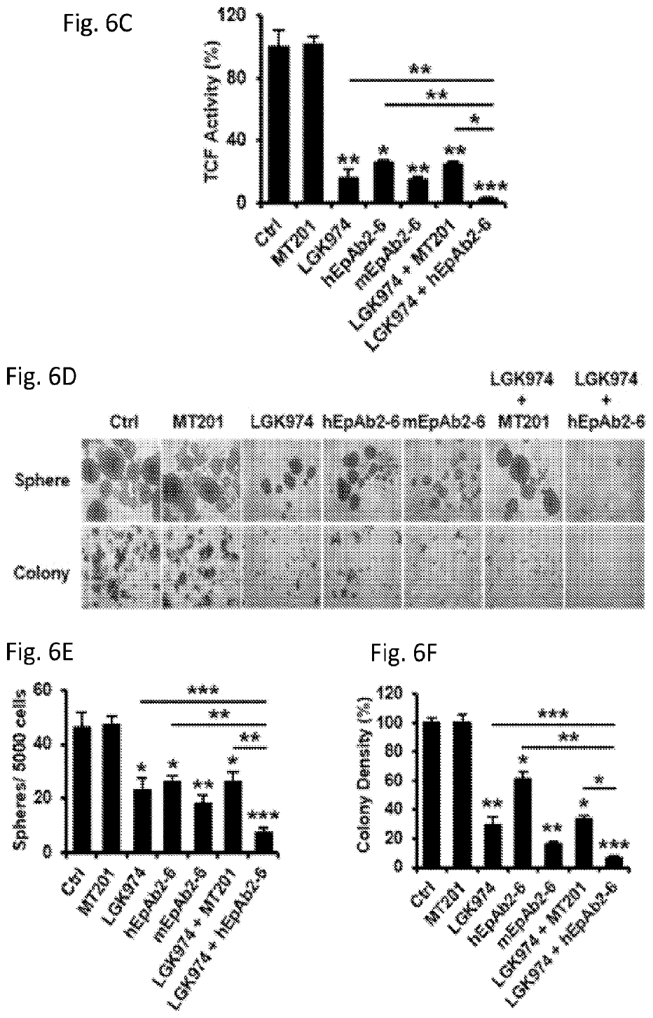

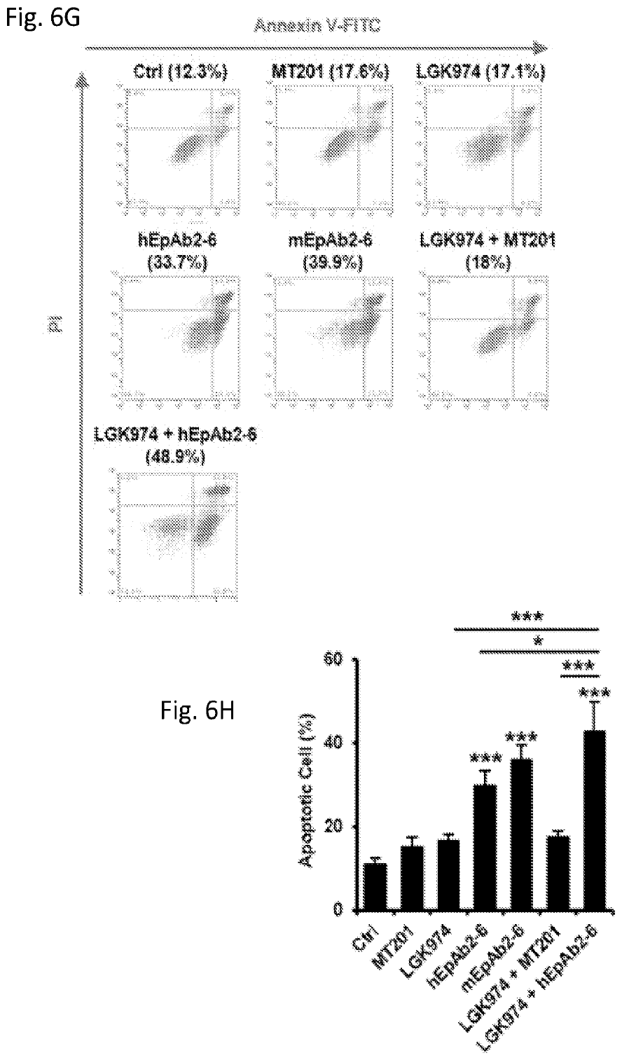

We then asked if inhibition of Wnt or EpCAM signaling in wild-type cells hinders nuclear translocation of β-Catenin. We decided not to block Wnt signaling by interfering with the β-Catenin destruction complex since we wanted to retain the ability of EpEX to activate Wnt-related signaling. Instead, we used LGK974, a porcupine inhibitor, which limits activation of Wnt ligands to prevent their receptor binding (Liu et al., 2013). In order to block EpCAM signaling, we used EpAb2-6, an anti-EpCAM monoclonal antibody, which functions by neutralizing EpEX to block its downstream signaling (Liao et al., 2015). Treatment with LGK974 decreased nuclear D3-Catenin but did not completely clear the protein from the nucleus. Similarly, treatment with EpAb2-6 also significantly decreased nuclear β-Catenin signal. Interestingly, the combination of LGK974 and EpAb2-6 almost abolished nuclear accumulation of the protein (FIG. 2G, FIG. 2H). These results were consistent with nuclear TCF activity and Axin2 expression data (FIG. 2I, FIG. 2J, FIG. 2K and FIG. 5), suggesting that EpEX can initiate Wnt signaling and cause translocation of β-Catenin to the nucleus. Furthermore, since the EpAb2-6 antibody (mEpAb2-6) was produced in mice by hybridoma technology, we decided to further test its humanized form (hEpAb2-6) (Liao et al., 2015); we also compared the effects of hEpAb2-6 with those of adecatumumab (MT201), a human anti-EpCAM antibody that was subjected to clinical trials. In this experiment, we found that hEpAb2-6 retained β-Catenin inhibitory activity thus related TCF activity, but MT201 did not show any significant effects compared to control-treated cells (FIG. 6A, FIG. 6B, FIG. 6C).

### 2.3 EpCAM Promotes Cancer Stemness and Tumorigenesis

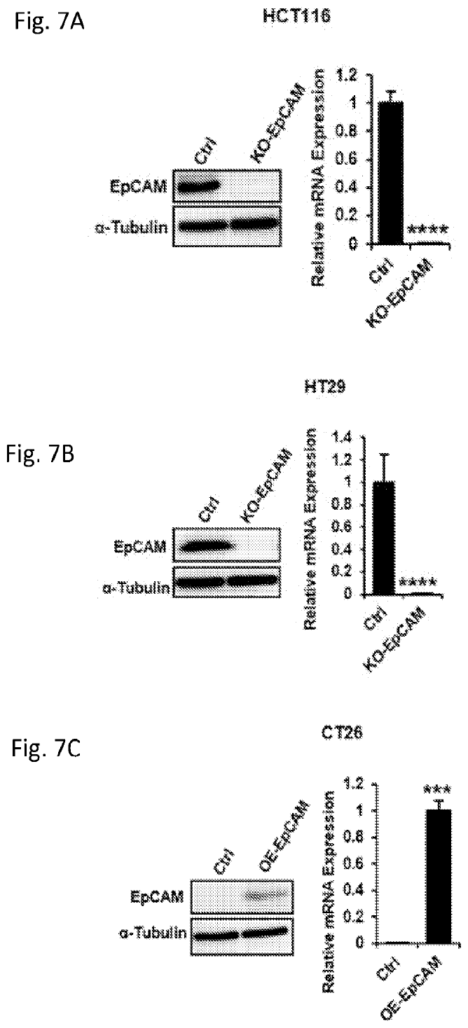

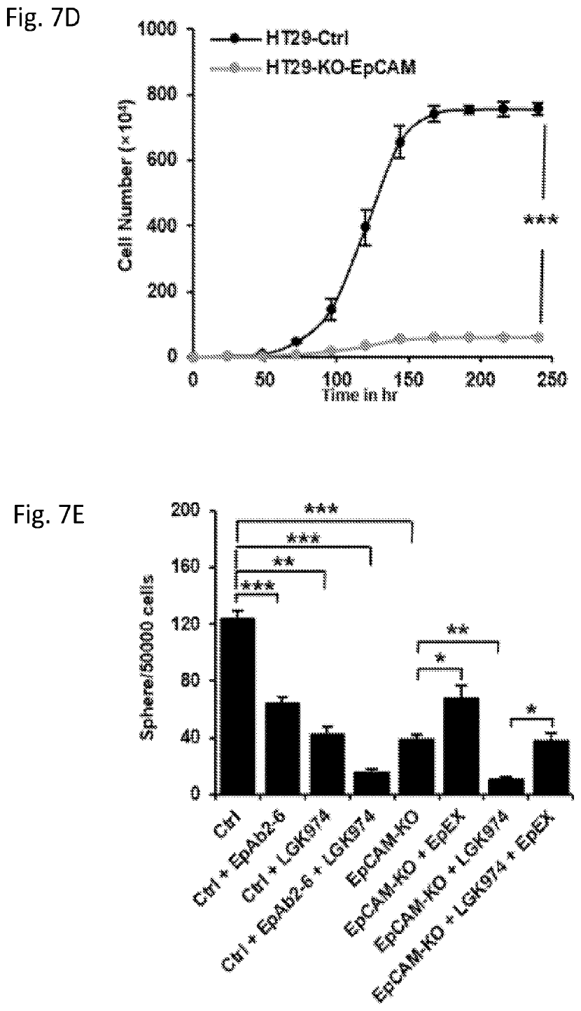

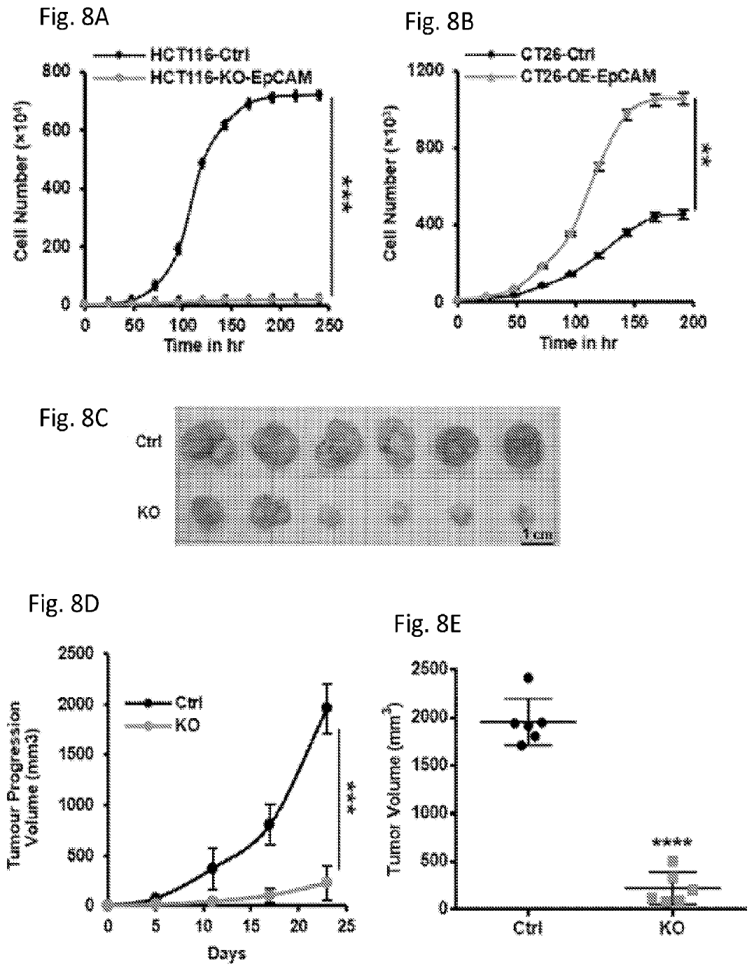

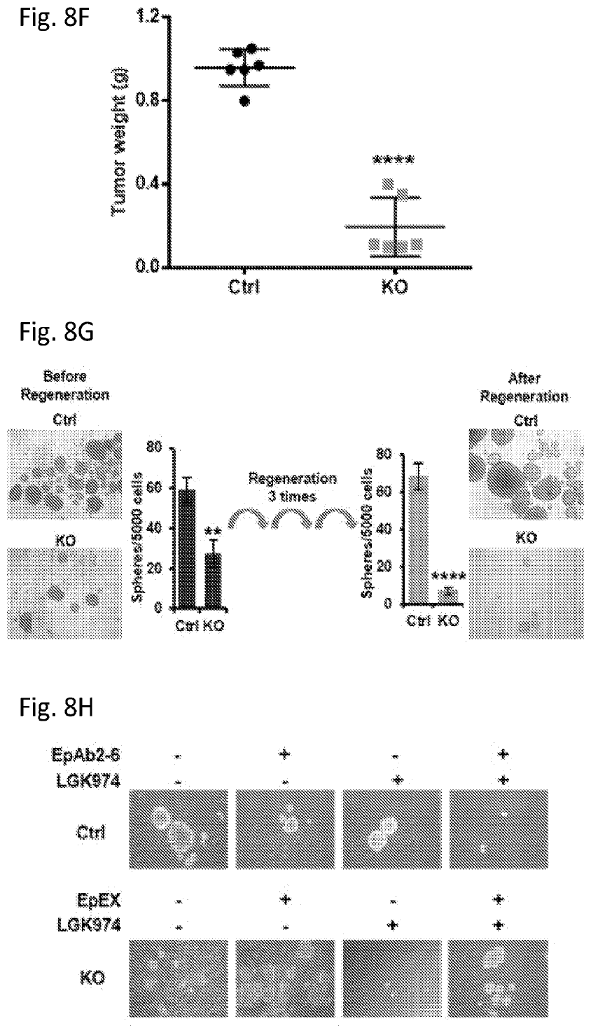

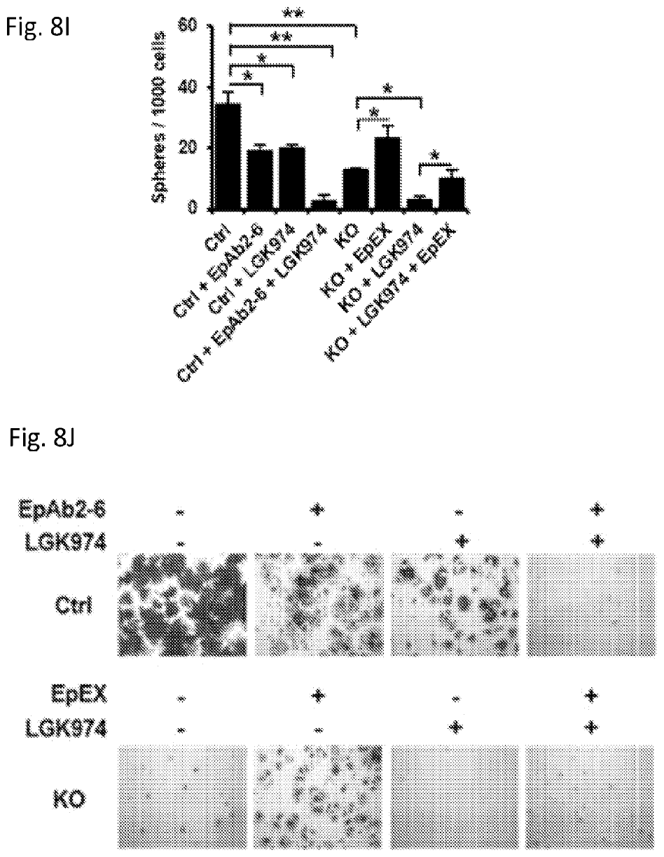

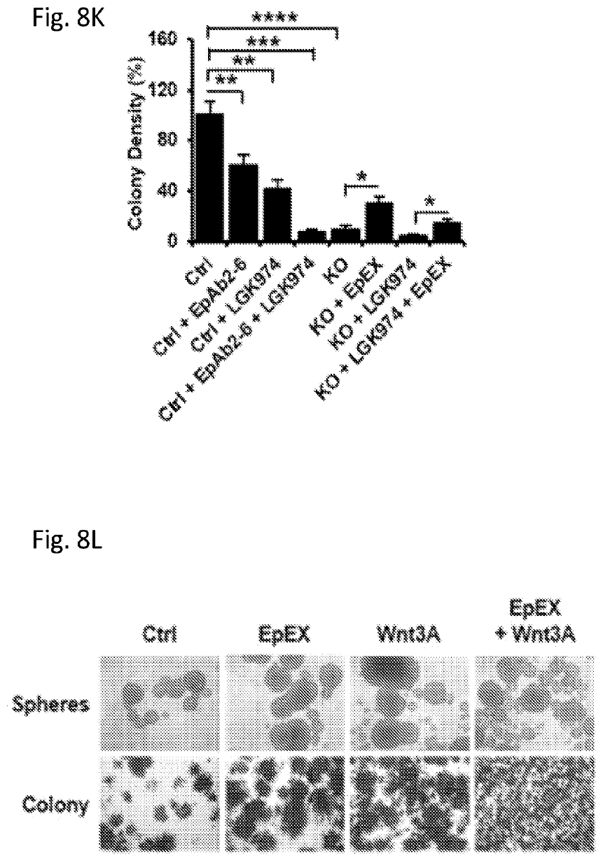

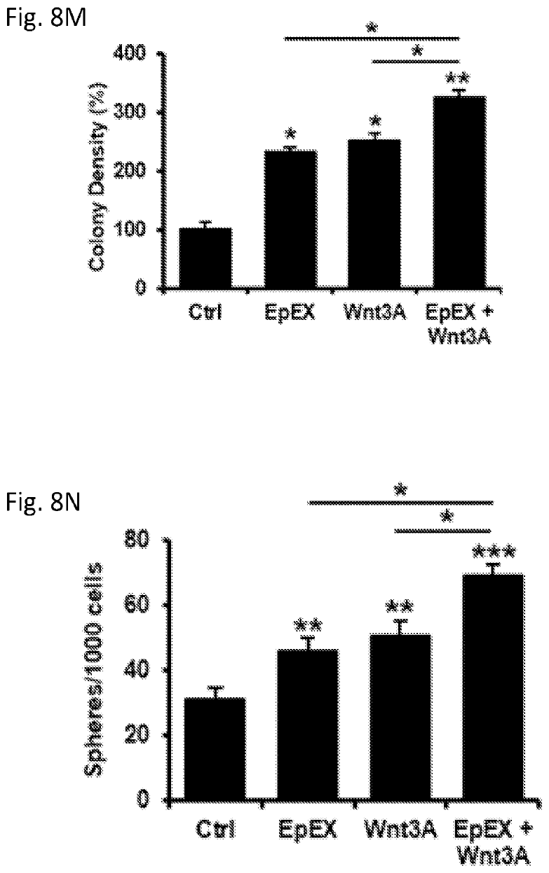

EpCAM is known to be abundantly expressed in CSCs whilst here we noticed that EpEX and EpICD might participate in Wnt-related signaling that is majorly involved in cancer stemness in many cancer types (Batlle and Clevers, 2017; Gires et al., 2020). Therefore, we next tested the functional role of EpCAM in promoting cancer cell proliferation and cancer stemness. To do so, we used CRISPR/Cas9 to produce EpCAM-knockout cells and forced the expression of EpCAM in CT26 cells that normally do not express EpCAM (FIG. 7A. FIG. 7B, FIG. 7C). Comparing the growth curves of control and EpCAM-knockout cells, we found that knocking out EpCAM significantly slowed growth, increasing cell-doubling time from 18±2 hours in controls to 51±2 hours in knockout HCT116 cells (FIG. 8A); likewise, the doubling time was increased from 23±2 hours in control to 48±2 hours in knockout HT29 cells (FIG. 7D). Furthermore, forced expression of EpCAM in CT26 cells decreased the doubling time from 30±2 hours in control cells to 21±2 hours in EpCAM-expressing cells (FIG. 8B). To evaluate tumorigenic potential of EpCAM in vivo, we subcutaneously transplanted as few as 10³ cells of control or EpCAM knockout cells into NSG mice. The EpCAM knockout cells exhibited a decreased tumor progression thus produced smaller tumors (FIG. 8C, FIG. 8D, FIG. 8E, FIG. 8F). Such tumorigenic potentials might be the consequences of cancer stemness exhibited by EpCAM. Thus, we conducted an in vitro regeneration assay with control and EpCAM knockout cells. After several passages, EpCAM knockout cells lost tumorigenic potential and produced smaller tumorsphere sizes and numbers (FIG. 8G). Since Wnt signaling also majorly govern cancer stemness, we sought to determine if EpCAM cross talks with Wnt pathway in order to attain such properties in cancer cells. Thus, we performed tumorsphere and colony formation assays while blocking either signaling or blocking them together. Treatment with either LGK974 or EpAb2-6 decreased tumorsphere and colony formation, while the combination almost completely ablated tumorspheres and colonies (FIG. 8H, FIG. 8I, FIG. 8J, FIG. 8K and FIG. 7E). On the other hand, EpCAM-knockout cells, which exhibited decreased ability to form tumorspheres or colonies, reverted to wild-type levels upon treatment with exogenous EpEX, suggesting that EpEX might be able to promote sternness through Wnt signaling. Interestingly, treating EpCAM-knockout cells with LGK974 caused a complete loss of ability to form spheres or colonies, but addition of EpEX with LGK974 could partially rescue sphere and colony formation (FIG. 8H, FIG. 8I, FIG. 8J, FIG. 8K and FIG. 7E). Thus, even in the absence of Wnt ligands, EpEX can promote some level of cancer sternness, might potentially be due to its involvements in Wnt signaling. Furthermore, treatment of exogenous Wnt3A or EpEX enhanced sphere and colony formation capacities, and the combination further amplified such potential (FIG. 8L, FIG. 8M, FIG. 8N). We then compared EpAb2-6 with MT201 in terms of the ability to inhibit colony and sphere formation, finding that MT201 shows no activity for modulating cancer sternness (FIG. 6D, FIG. 6E, FIG. 6F). Altogether, these data support the idea that EpCAM and Wnt proteins coordinately stimulate (3-Catenin signaling and promote cancer sternness in CRC.

2.4 EpEX Interacts with Wnt Receptors to Promote β-Catenin Signaling

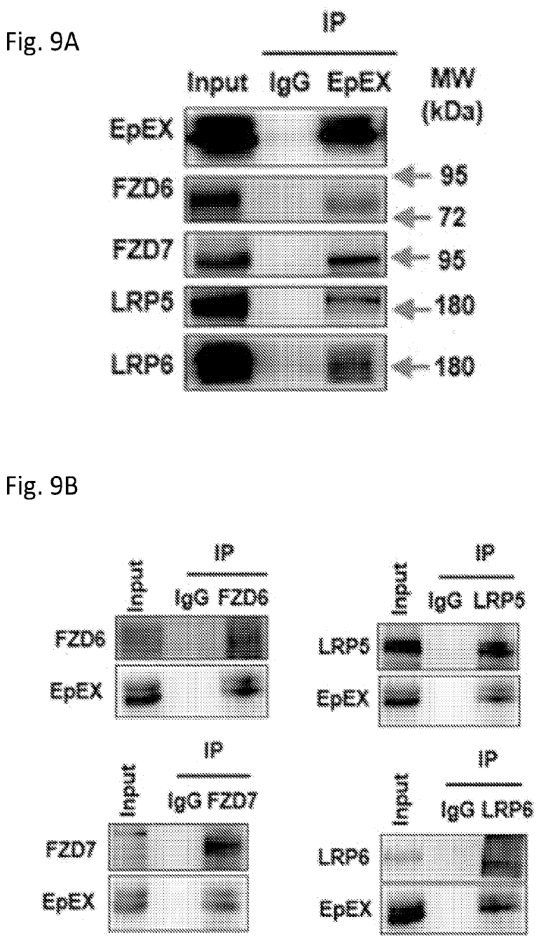

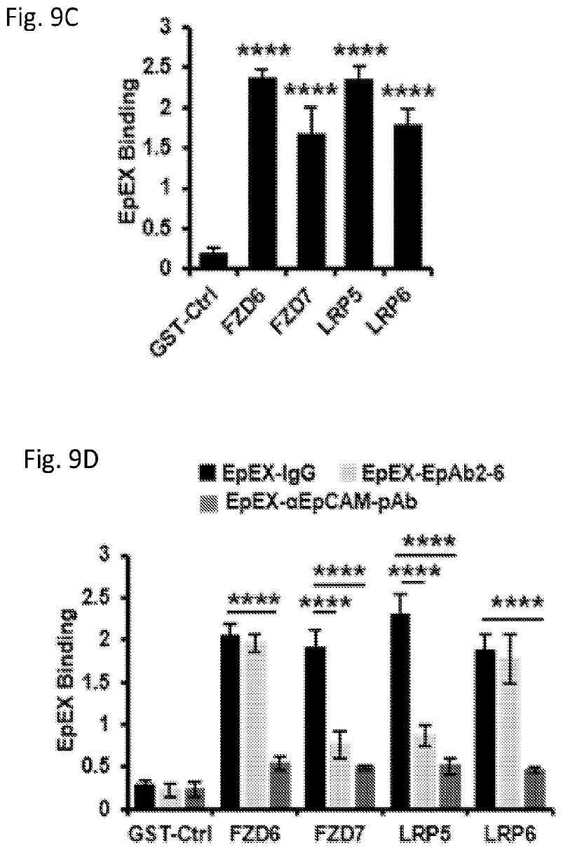

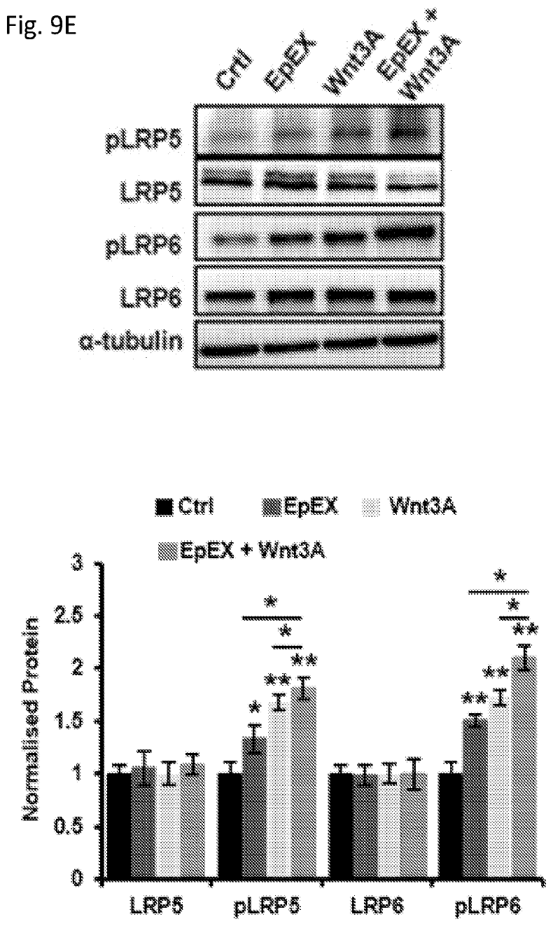

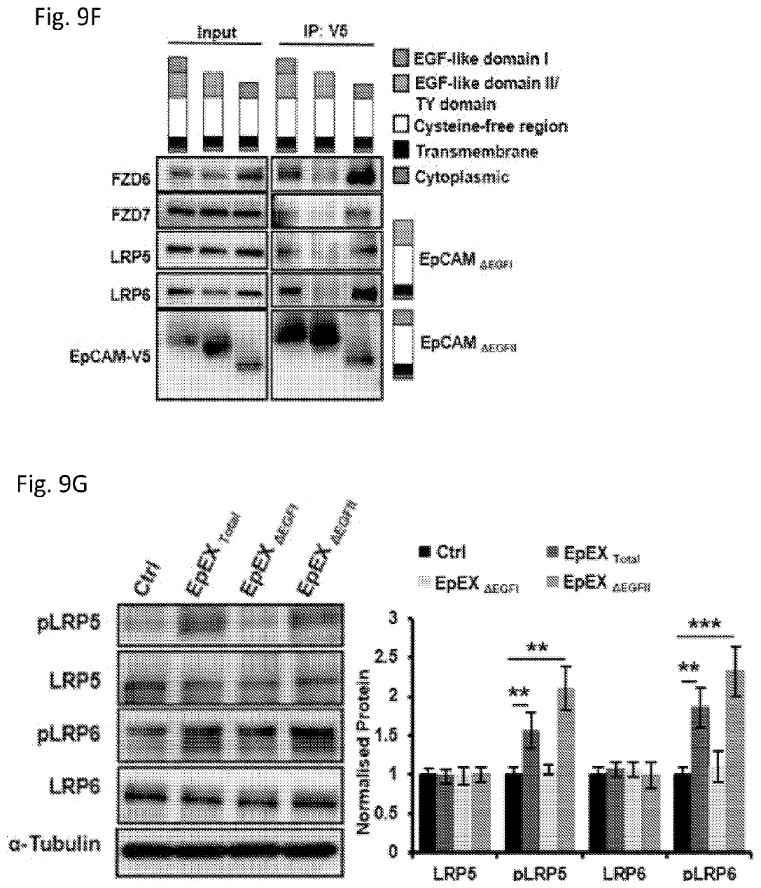

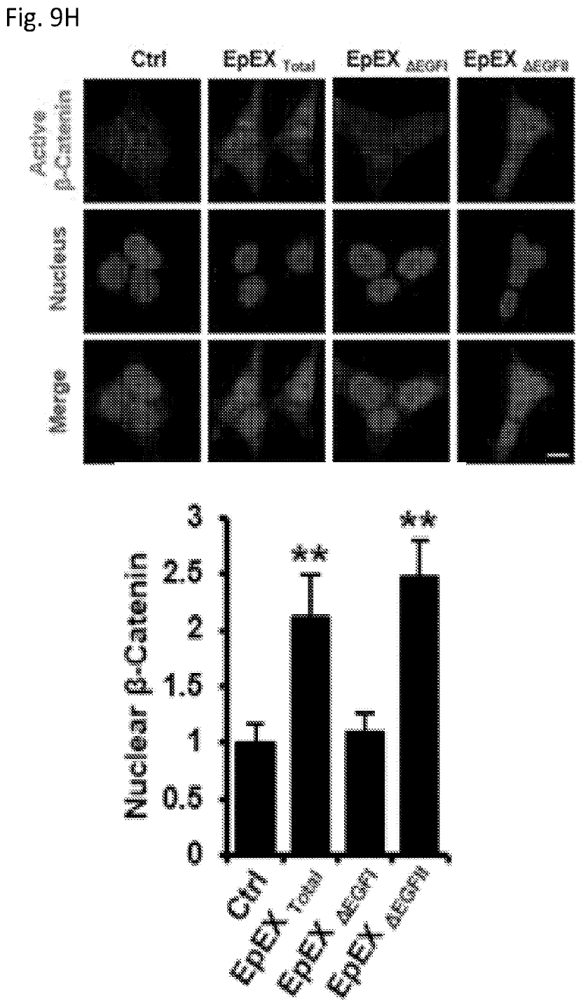

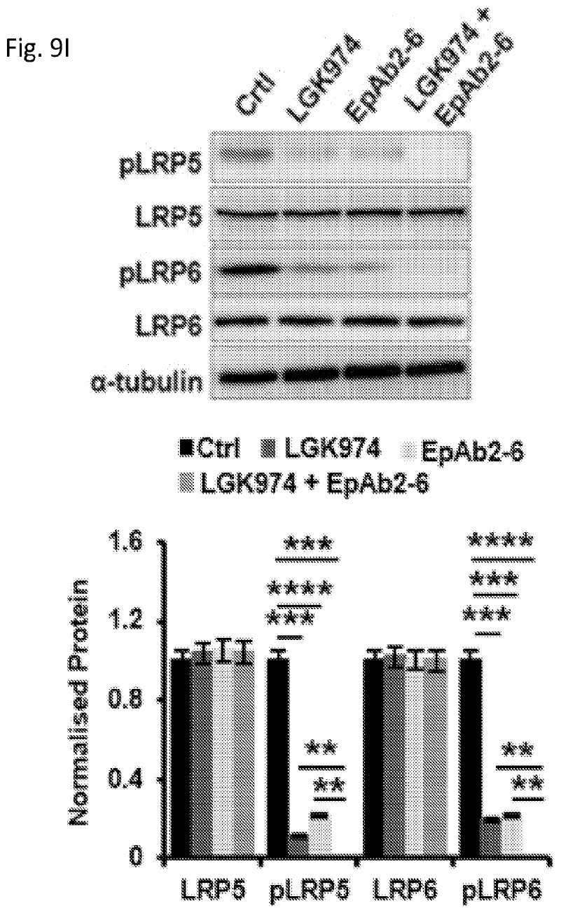

Since we determined that EpEX could activate Wnt signaling, we further probed the interaction of EpEX with Wnt receptors. We co-immunoprecipitated EpEX or Wnt receptor molecules, FZD6/7 and LRP5/6, and subjected the pulled-down products to western blot analysis. The results demonstrated that EpEX forms complexes with Wnt receptor proteins (FIG. 9A, FIG. 9B). In order to confirm EpEX binding to Wnt receptor proteins, we coated ELISA plates with purified FZD6/7 or LRP5/6 fusion proteins (with GST-tag) and tested if EpEX can bind to the proteins (FIG. 9C). While EpEX was found to bind with all receptor proteins, pre-incubation of EpEX with an anti-EpCAM polyclonal antibody (almost blocks all epitopes) significantly decreased such binding. Moreover, pre-incubation of EpEX with EpAb2-6 substantially decreased binding to only FZD7 and LRP5 proteins, suggesting that the EpAb2-6 epitope on EpEX might be involved in binding to FZD7 and LRP5 (FIG. 9C, FIG. 9D). In this context, in Wnt pathway, the receptor-ligand interaction initiates signaling by recruiting the β-Catenin destruction complex, which activates the molecule and allows it to translocate to the nucleus. In this process, LRP5/6 is phosphorylated by Glycogen Synthase Kinase 3P (GSK3P) or Casein Kinase 1 (CKT) at the cell membrane that are present in the destruction complex (Nusse and Clevers, 2017). Thus, we tested if interaction of EpEX with Wnt receptors could initiate such phosphorylation. Indeed, treatment of exogenous EpEX or Wnt3A increased LRP5/6 phosphorylation, and the combination produced an augmented effect (FIG. 9E).

These results further encouraged us to evaluate which specific domain of EpEX interacts with Wnt receptors. To answer this question, we transfected HEK293 cells with plasmids expressing deletion mutants for either EGF-like domain-I- or domain-II-deleted EpEX, and performed immunoprecipitation of EpEX (FIG. 9F). The results revealed that EGF-like domain I of EpEX directly interacts with the Wnt receptors. Moreover, since we previously observed EpEX could induce phosphorylation of LRP5/6 (FIG. 9C) and nuclear translocation of β-Catenin (FIG. 2A, FIG. 2B, FIG. 3B, FIG. 3C, FIG. 3E and FIG. 4A, FIG. 4B); we tested whether domain I of EpEX binding to Wnt receptors could induce the same effects. Therefore, we treated the cells with EGF-like domain-(I/IJ)-deleted mutant EpEX proteins to observe their activity (FIG. 9G, FIG. 9H). Indeed, we noticed that EpEX-domain I mutant protein treatment could induce phosphorylation of LRP5/6 and nuclear translocation of β-Catenin, whereas EpEX-domain II mutant protein treatment did not produce the same effects. As we previously observed EpAb2-6 and LGK974 could attenuate nuclear translocation of β-Catenin (FIG. 2G, FIG. 2H), we next tested whether such treatment could inhibit phosphorylation of LRP5/6 in order to block Wnt signaling. We found that either LGK974 or EpAb2-6 treatment could decrease LRP5/6 phosphorylation, and the combined treatment resulted in absolute abrogation of such phosphorylation (FIG. 9I). These results confirm that the EGF-like domain I of EpEX directly interacts with Wnt receptors to activate β-Catenin signaling.

### 2.5 EpEX and Wnt Activate TACE and γ-Secretase

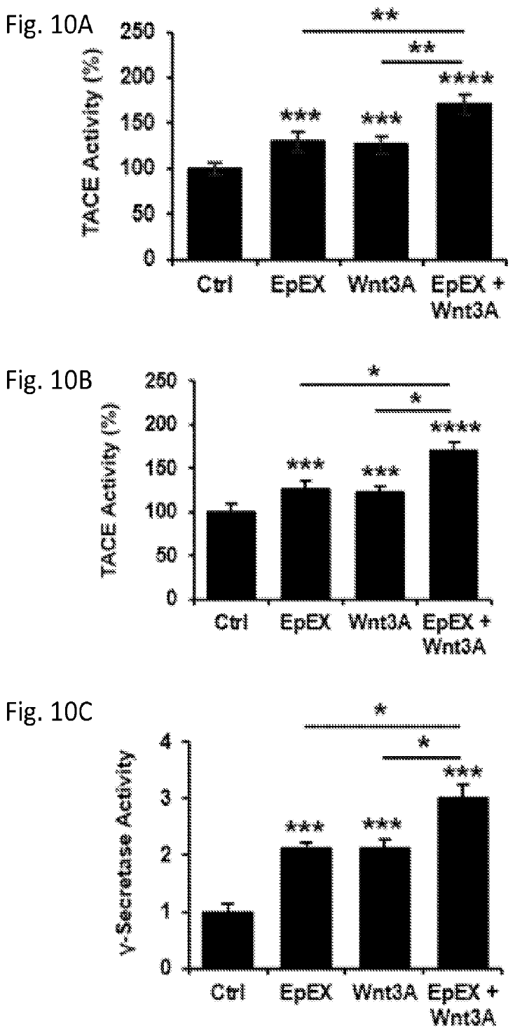

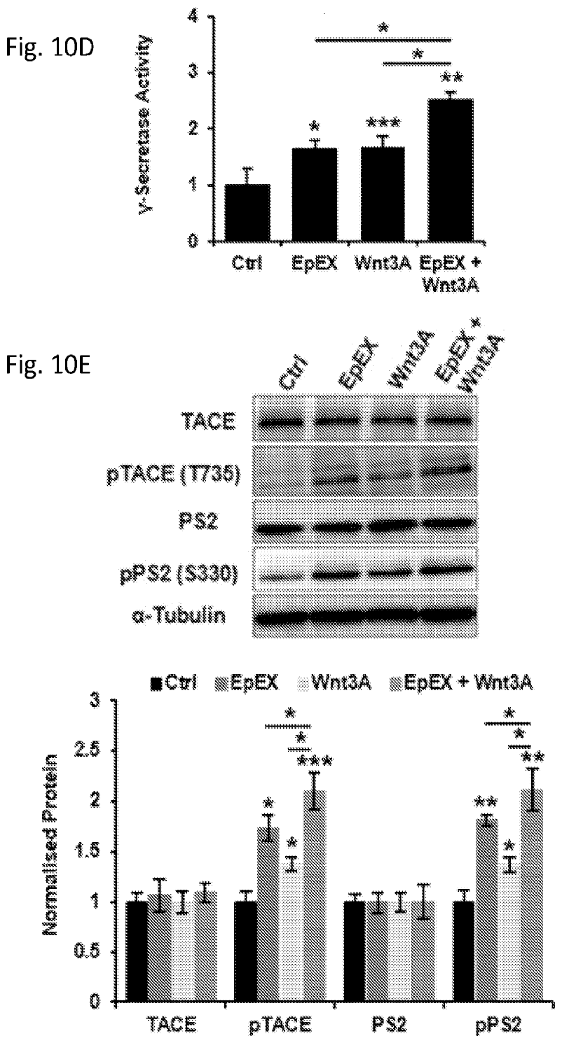

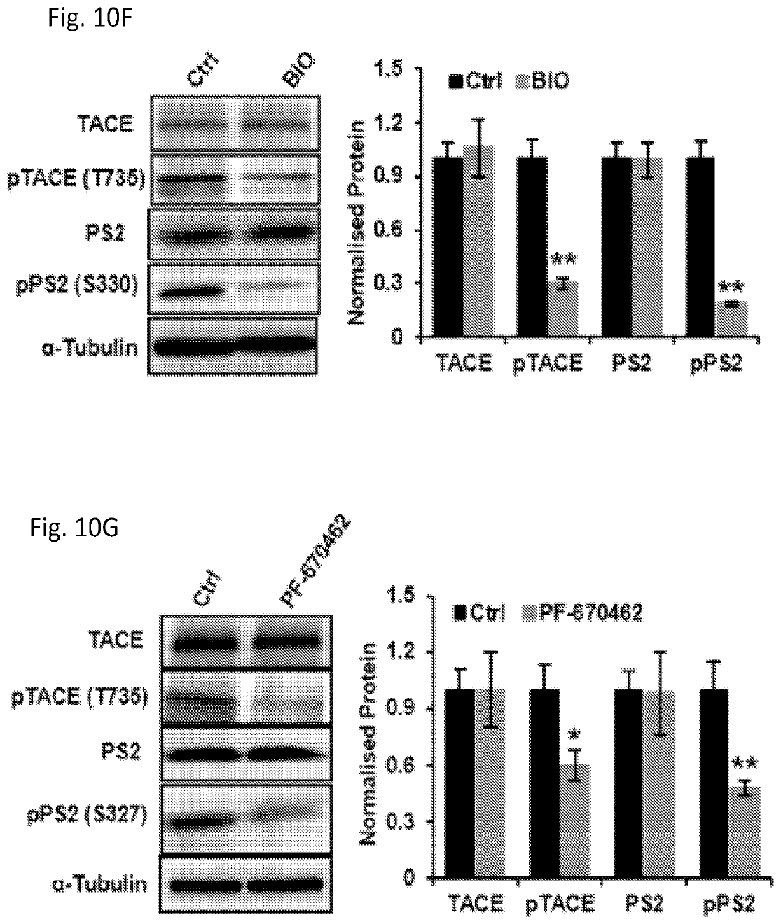

Since we found that EpEX interacts with Wnt receptors, we wanted further exploring the factors that could influence production of EpEX and thus EpICD further. Therefore, we asked whether EpEX-induced Wnt signaling could activate TACE and γ-secretase that cleave EpEX and EpICD respectively. Interestingly, we found that treatment with exogenous EpEX or Wnt3A enhanced TACE and γ-secretase activities, and the combination further augmented such activation (FIG. 10A, FIG. 10B, FIG. 10C, FIG. 10D). Regarding the mechanism of the upregulated activity, we found that Wnt3A and EpEX treatment increases the phosphorylation of TACE and presenilin-2 (PS2), the activating subunit of γ-secretase (FIG. 10E). In order to identify the kinases involved in this process, we blocked GSK3 or CK1 of β-Catenin destruction complex with small molecule inhibitors and observed decreased phosphorylation of TACE and PS2, suggesting GSK3 and CK1 are involved in the process (FIG. 10F, FIG. 10G). These observations warrant further investigations to identify the detailed mechanisms of activation of TACE and γ-secretase by the activation of Wnt pathway.

### 2.6 EpICD Upregulates Wnt Receptor Protein Expressions

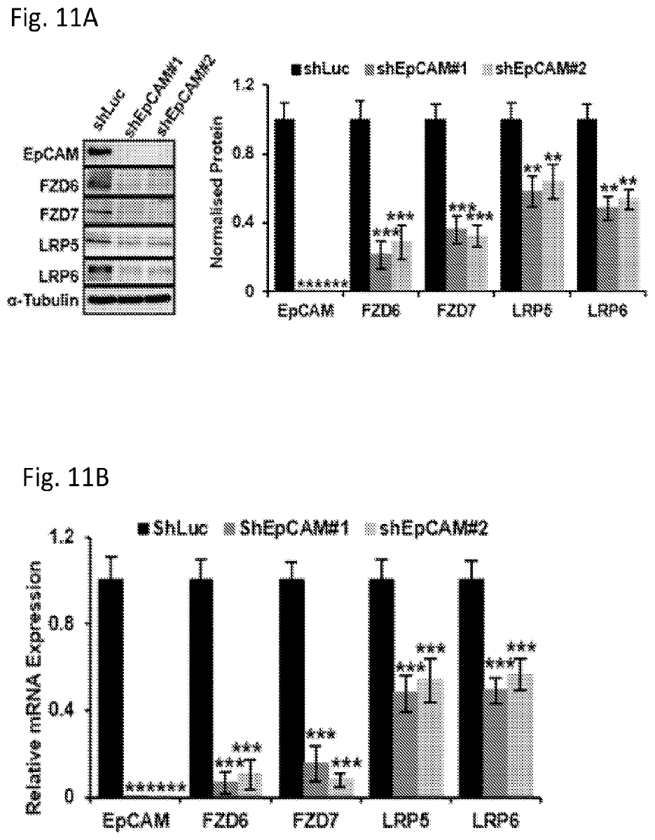

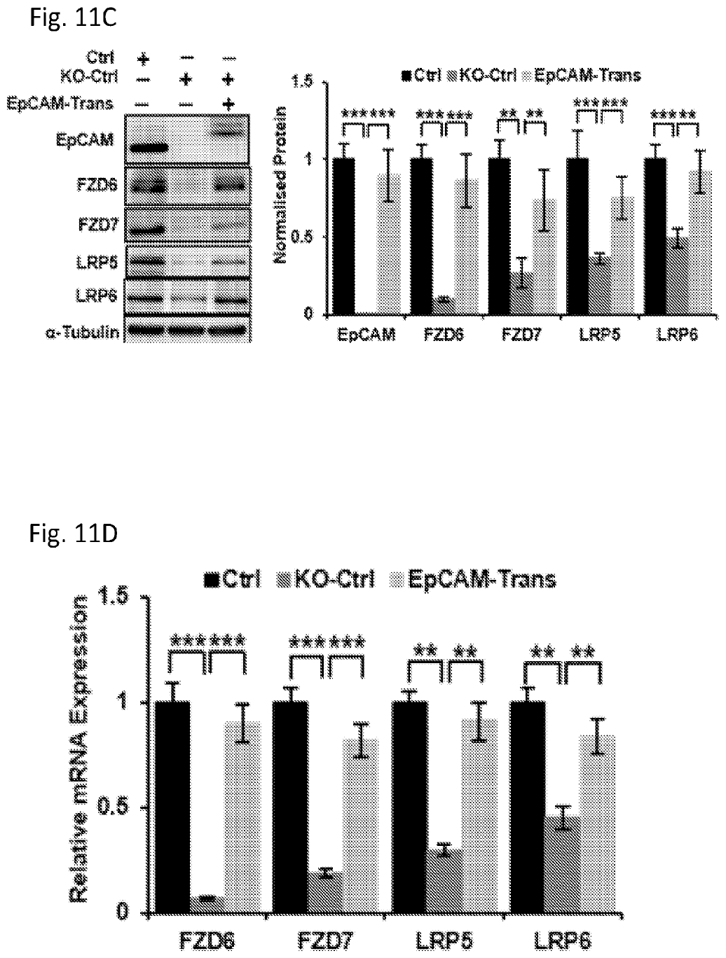

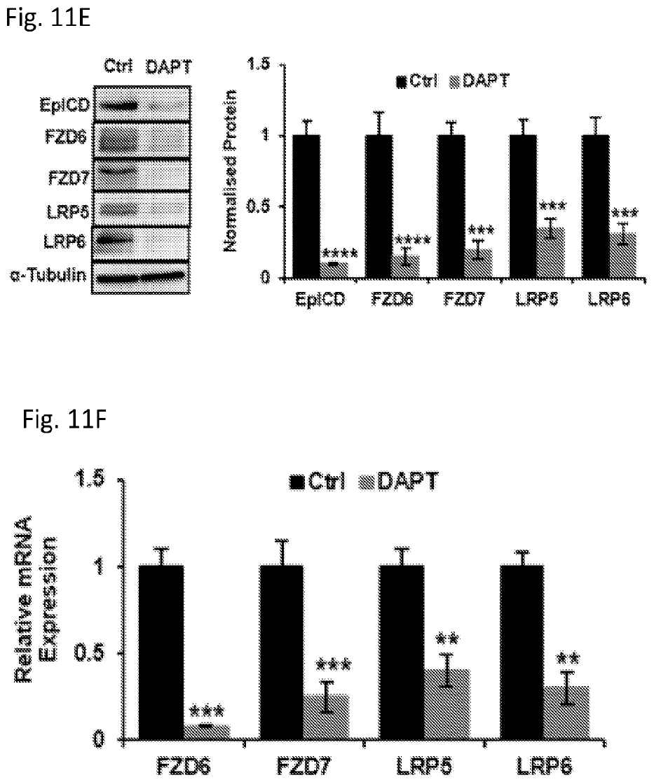

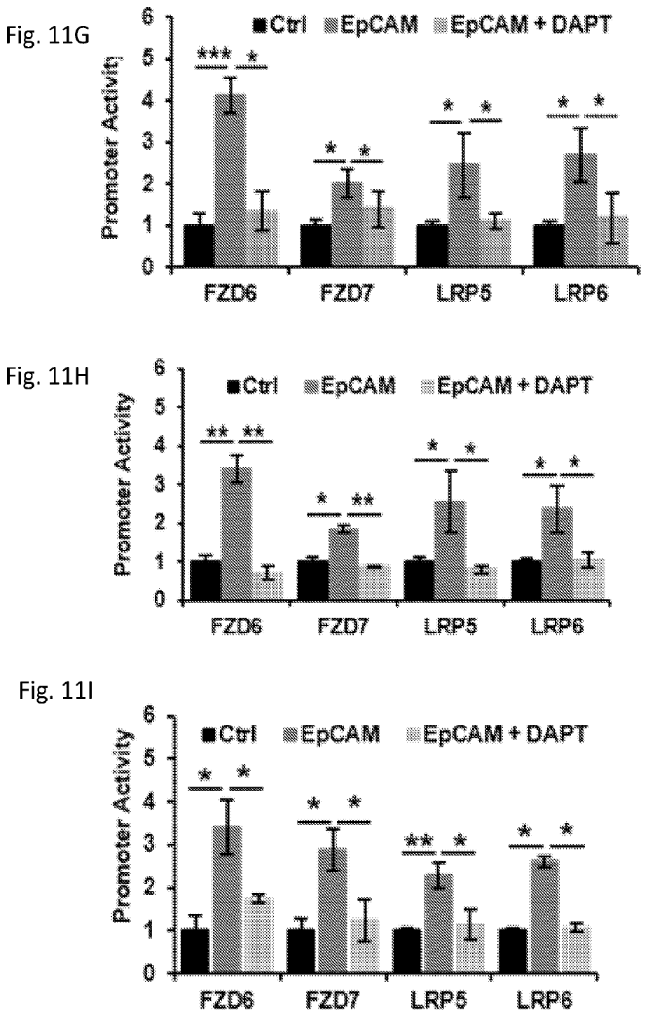

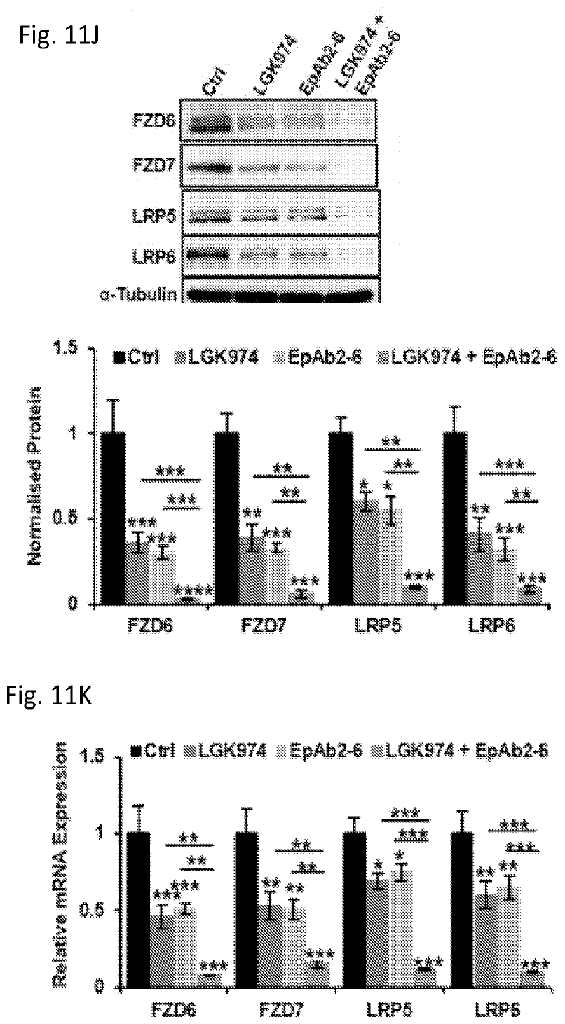

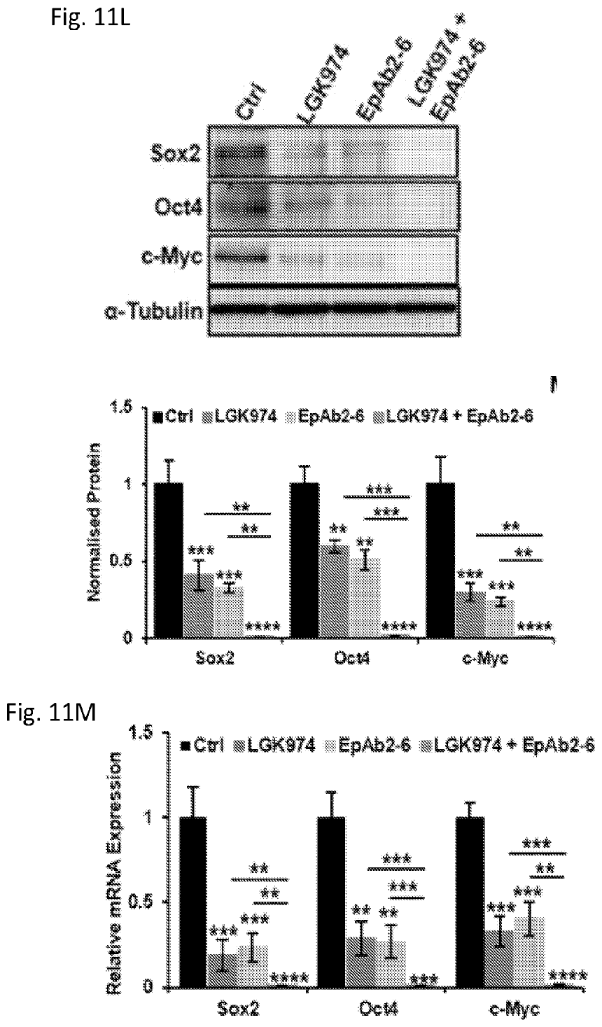

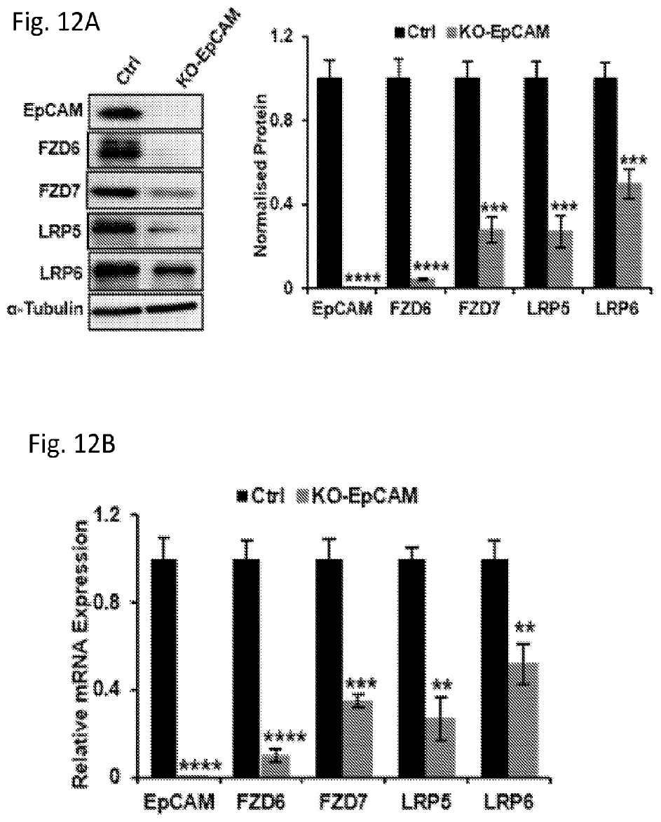

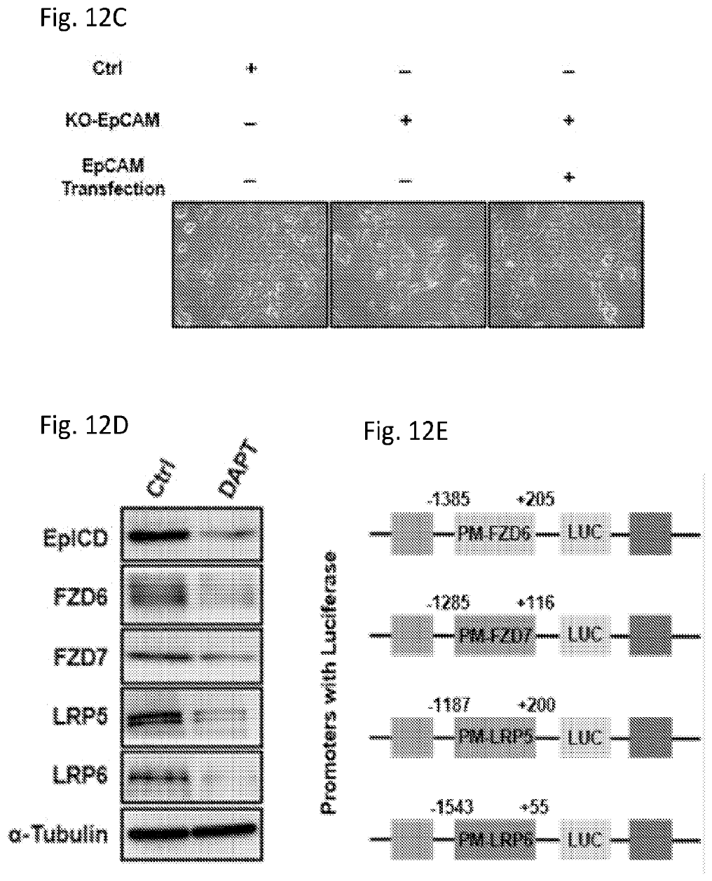

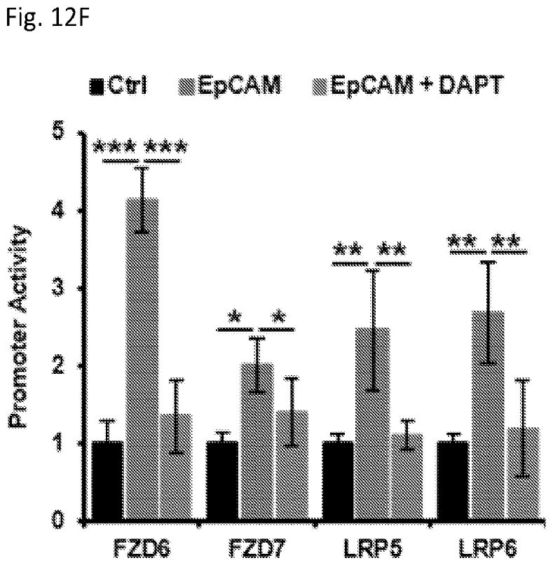

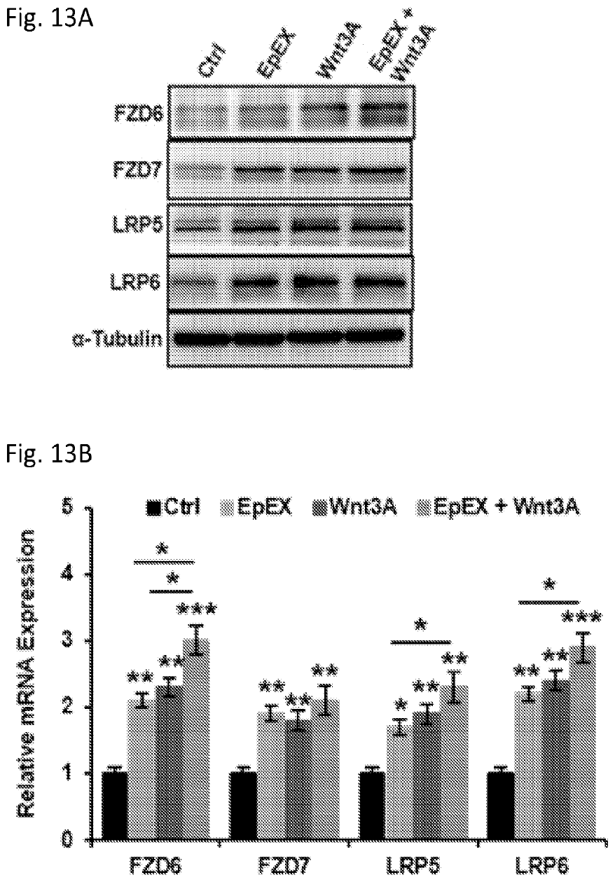

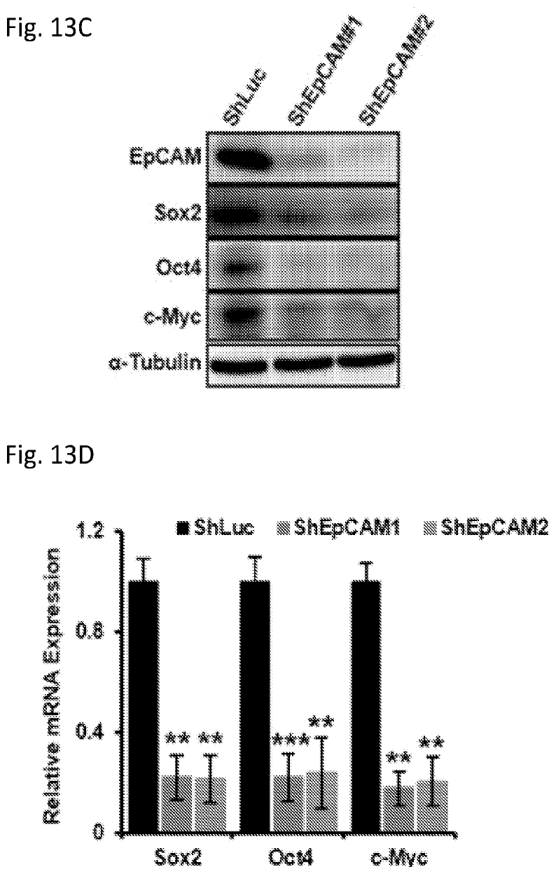

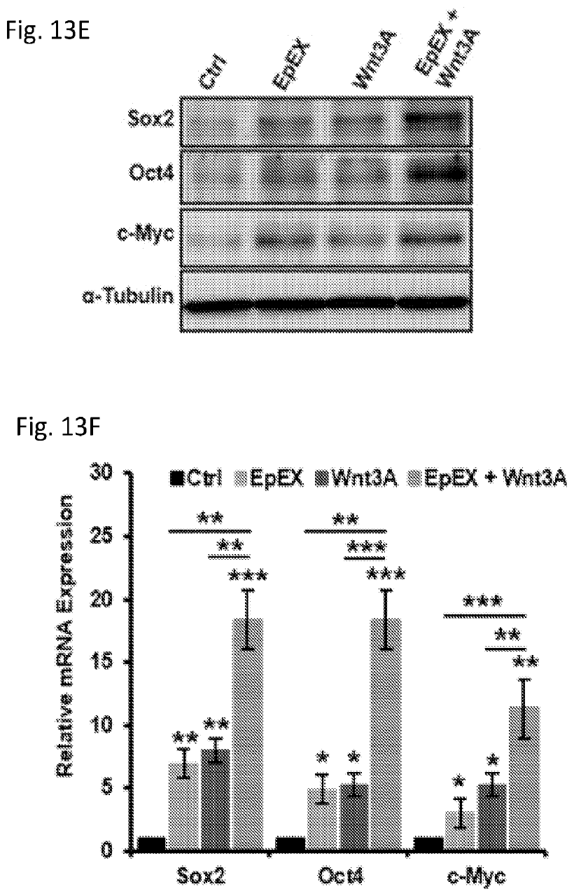

High levels of Wnt receptor proteins may increase Wnt activity (MacDonald and He, 2012), thus affecting cancer sternness. Therefore, we asked whether the levels of Wnt receptor proteins are affected by EpCAM signaling. Interestingly, we found that knockout or knockdown of EpCAM significantly decreased Wnt receptor protein levels (FIG. 11A, FIG. 11B, and FIG. 12A, FIG. 12B). Furthermore, transfecting knockout cells with wild-type EpCAM plasmid rescued Wnt receptors and changed into wild-type like cell morphology (FIG. 1C, FIG. 11D and FIG. 12C). Further blocking the shedding of EpICD with DAPT, a γ-secretase inhibitor, we observed reduced expression of Wnt receptors (FIG. 11E, FIG. 11F and FIG. 12D). Based on these results, we postulated that EpICD might function as a transcription factor to promote expression of Wnt receptors. To test this hypothesis, we constructed luciferase reporters under the control of Wnt receptor promoters (FIG. 12E). As predicted, transfecting cells with EpCAM led to enhanced promoter activity, whereas DAPT treatment almost completely blocked the effect (FIG. 11G, FIG. 11H, FIG. 11I and FIG. 12F). These data suggest that EpICD upregulates Wnt receptor protein expression levels via direct interactions with their promoters. In this context, overproduction of EpEX (as in cancer cells) may phosphorylate presenilin-2 via EpEX-EGFR-ERK axis in order to activate γ-secretase that cleaves EpICD (Chen et al., 2020; Liang et al., 2018). In this study, we also noticed that both Wnt and EpEX could activate γ-secretase in order to produce more EpICD (FIG. 10). Thus, we sought to investigate if EpEX could upregulate Wnt receptors. Indeed, EpEX and Wnt3A treatment upregulated expressions of Wnt receptors and the combination further augmented the phenomena in both protein and mRNA level (FIG. 13A, FIG. 13B). Thus, EpAb2-6 and LGK974 could each partially decrease whilst their combination almost nullified expressions of Wnt receptors (FIG. 11J, FIG. 11K). Furthermore, pluripotency factors such as Oct4, Sox2 and c-Myc are thought to be critical for cancer sternness, and transcription of these genes is well studied to be activated by EpICD (Lin et al., 2012), thus in this study knocking down EpCAM decreased the protein and relative mRNA expression levels of sternness factors (FIG. 13C, FIG. 13D). As sternness factors are direct targets of the Wnt pathway, treating the cells with either EpEX or Wnt3A induced expressions of pluripotency factors while the combination treatment further enhanced the effect (FIG. 13E. FIG. 13F). In fact, treating the cells with LGK974 or EpAb2-6 decreased pluripotency factor expressions, and the combined treatment completely nullified such activity (FIG. 11L, FIG. 11M). These results are in agreement with the previous work by Lin et al (Lin et al., 2012), which revealed that EpICD functions as a transcriptional regulator of sternness proteins. Therefore, it appears that EpEX binds to Wnt receptors to initiate signaling, while EpICD may function as a transcription factor to drive production of Wnt receptor proteins and sternness factors attaining cancer-stemness.

### 2.7 LGK974 and EpAb2-6 Cooperatively Induce Apoptosis and Inhibit Tumor Progression

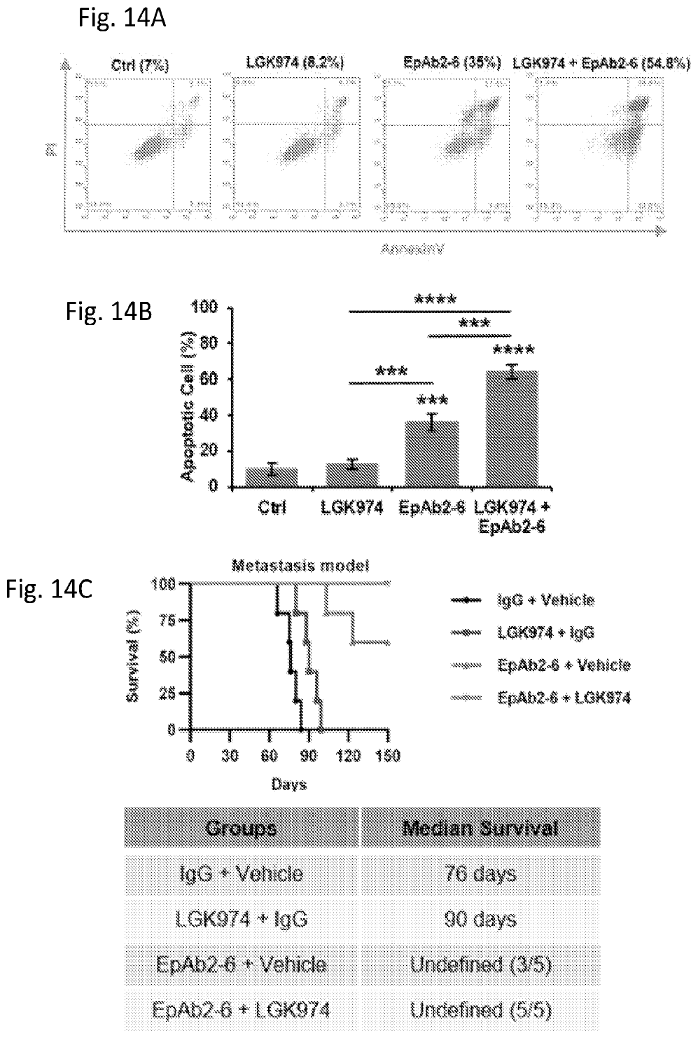

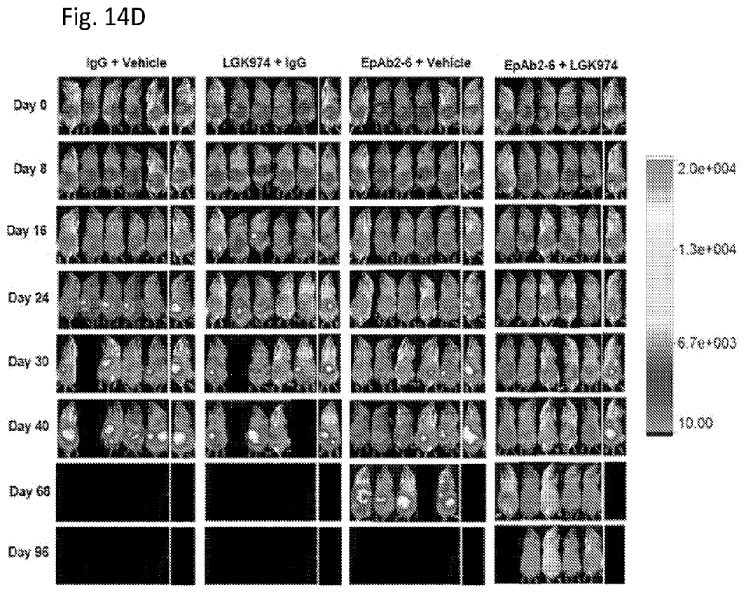

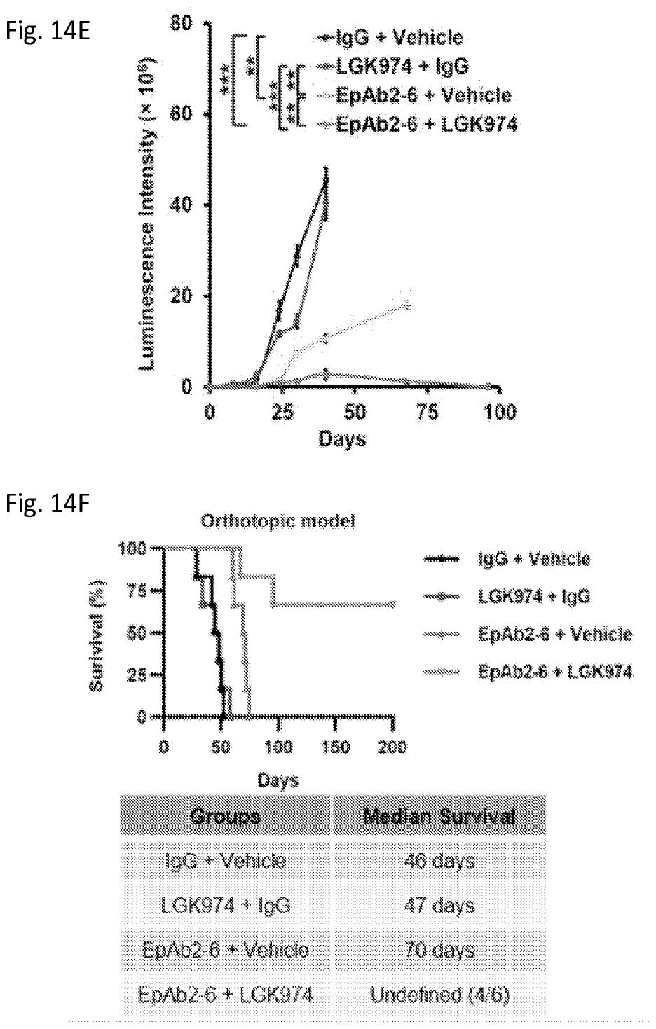

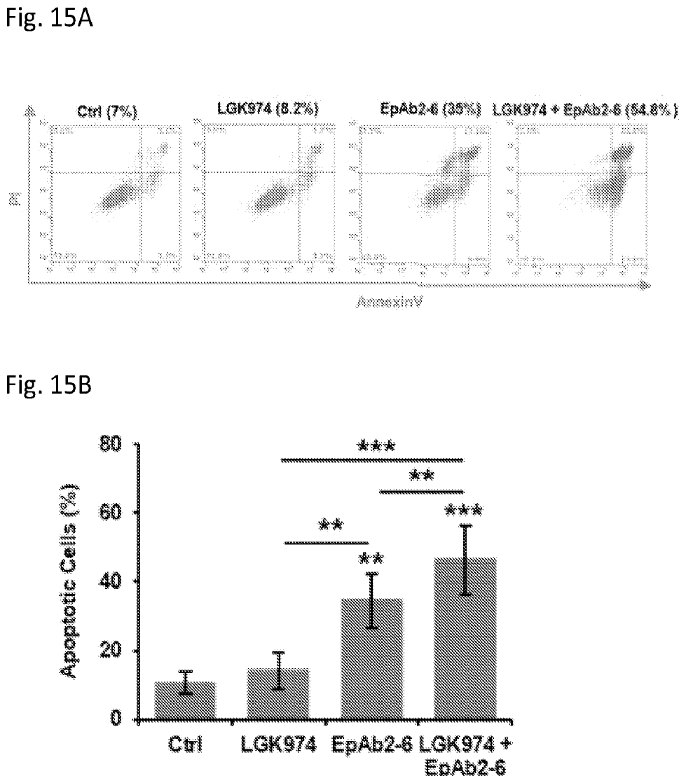

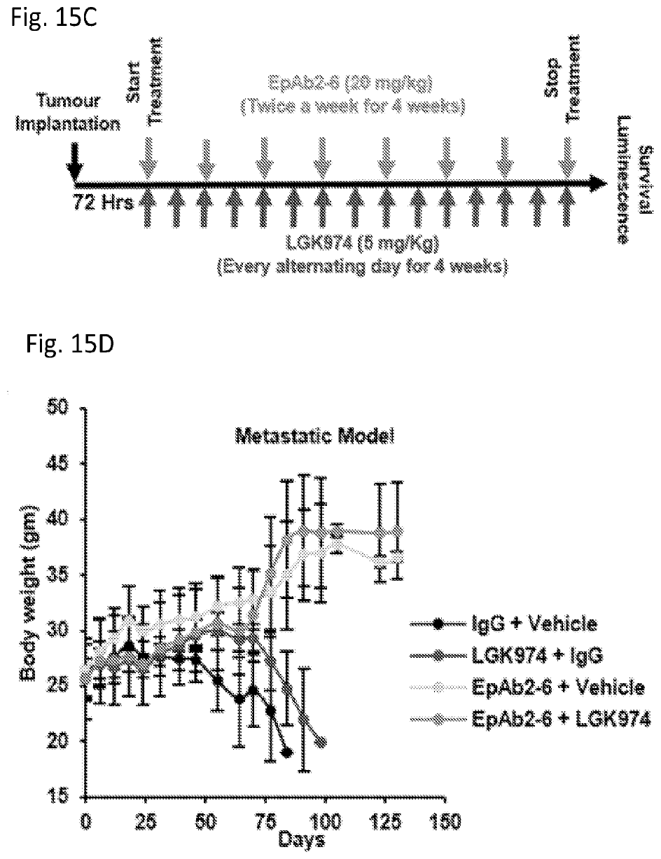

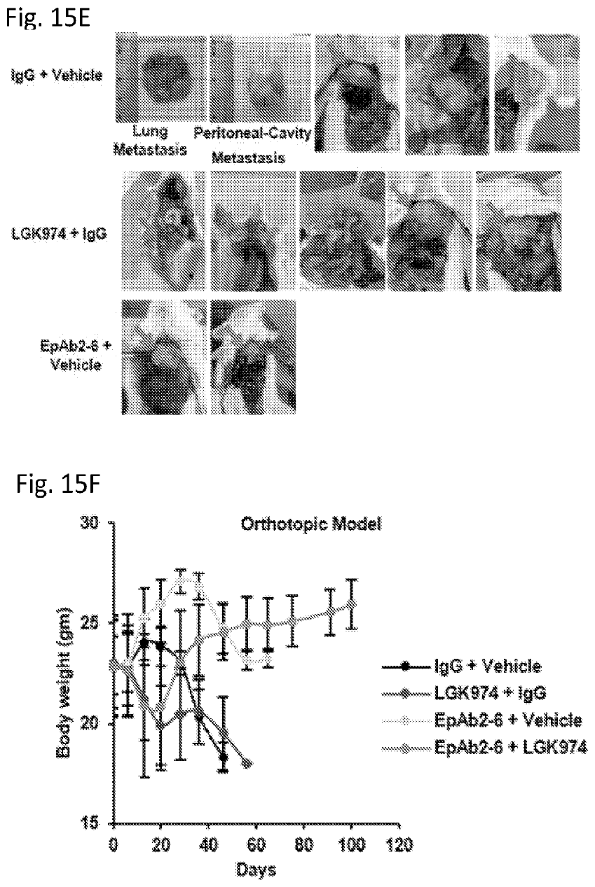

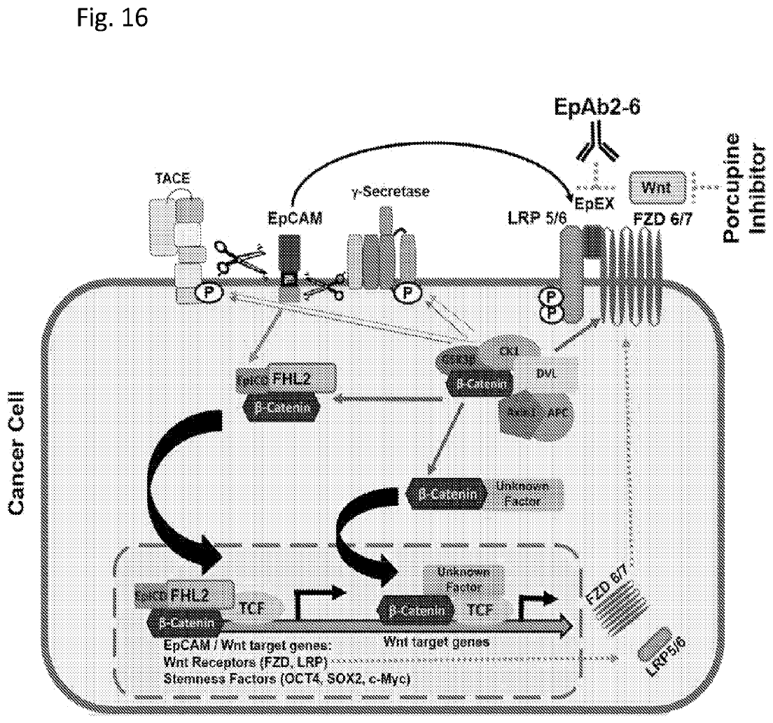

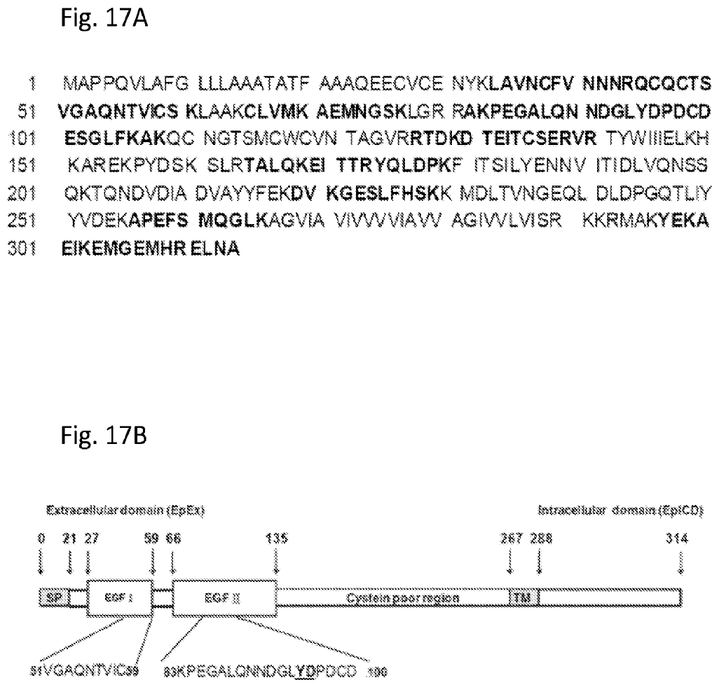

Our data to this point suggested that EpCAM and Wnt proteins coordinately stimulate Wnt signaling to promote stemness that could be inhibited by simultaneous blocking of both signals using EpAb2-6 and LGK974, thus we tested the cellular effects of the combination. We found that treatment of EpAb2-6 alone could induce apoptosis in colon cancer cells, but LGK974 could not. However, the induction of apoptosis was amplified in cells receiving combination treatment (FIG. 14A, FIG. 14B and FIG. 15A, FIG. 15B). We further evaluated if such effects could be reproduced with MT201 antibody and discovered that the antibody does not have such activity, while hEpAb2-6 exhibited similar activity as mEpAb2-6 (FIG. 6G, FIG. 6H). These results encouraged us to test the anti-tumor effects of EpAb2-6 in animal models. In this context, EpCAM was previously reported to augment EMT gene expression that promotes metastasis in colon cancer (Lin et al., 2012). Therefore, we decided to evaluate the combined effects of EpAb2-6 and LGK974 in human both metastatic and orthotopic animal models. For the metastatic animal model, we injected HCT116 cells via the tail vein, while in the orthotopic model, cells were surgically transplanted into the cecum wall of the animal. For both models, treatments were initiated 72 h post-transplantation (FIG. 15C). In the metastatic model, we found that treatment with either EpAb2-6 or the combination could prolong animal survival. Only 2 out of 5 mice in the EpAb2-6 group and none out of 5 in the combination group died by the end of the study. However, most of the animals in control IgG- or LGK974-treated groups were found to have distant metastases, which was associated with decreased overall survival (FIG. 14C and FIG. 15D, FIG. 15E). Similarly, in the orthotopic model, all animals in the control IgG and LGK974 groups developed significant tumors and displayed low median survival (FIG. 14D, FIG. 14E, FIG. 14F). The EpAb2-6-treated group had much slower tumor progression and showed relatively higher median survival than that of the control-IgG or LGK974 treated groups. The reduction in tumor progression was even more pronounced in the combination treatment group; 4 out of 6 animals were found to be completely free of tumors, and the overall survival of the animals was prolonged (FIG. 14D, FIG. 14E, FIG. 14F). Of note, previous studies reported LGK974 is non-toxic at a dose of 5 mg/kg body weight (Liu et al., 2013). We noticed the body weights of both LGK974-treated and the combination-treated animals decreased during the treatment period (FIG. 15F). However, after cessation of treatment, the combination group recovered body weight, while the LGK974-treated mice continued to lose body weight, probably due to the tumor burden. Taking these data together, we conclude that EpCAM actively mechanizes Wnt machinery via EpEX and EpICD in order to establish cancer sternness in CRC therefore the combination treatment of EpAb2-6 and a porcupine inhibitor may fully suppress cancer sternness to maximize therapeutic efficacy (FIG. 16).

### 2.8 EpAb2-6 Binds to the EGF-Like Domains I and II of EpCAM

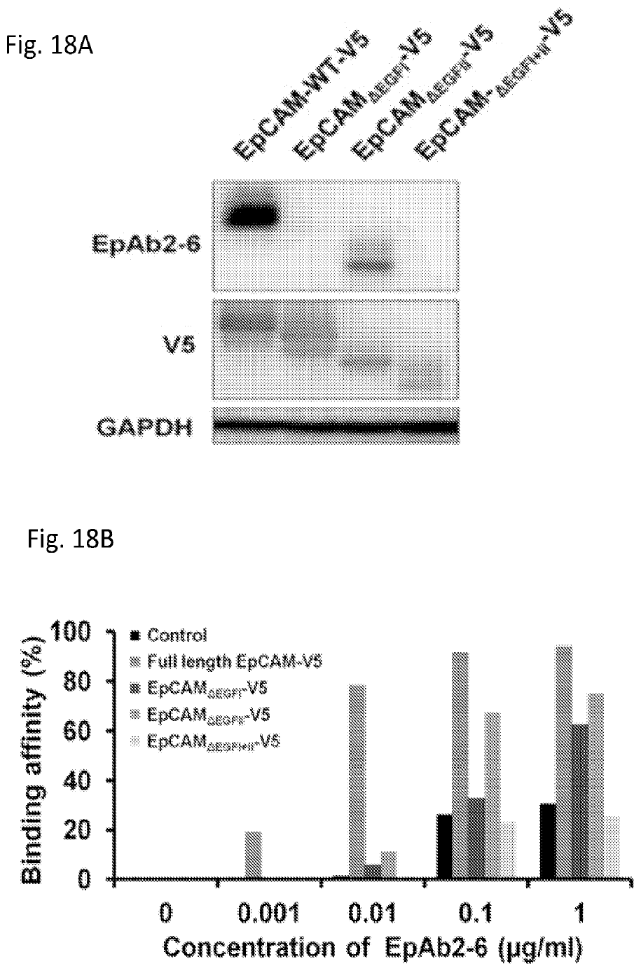

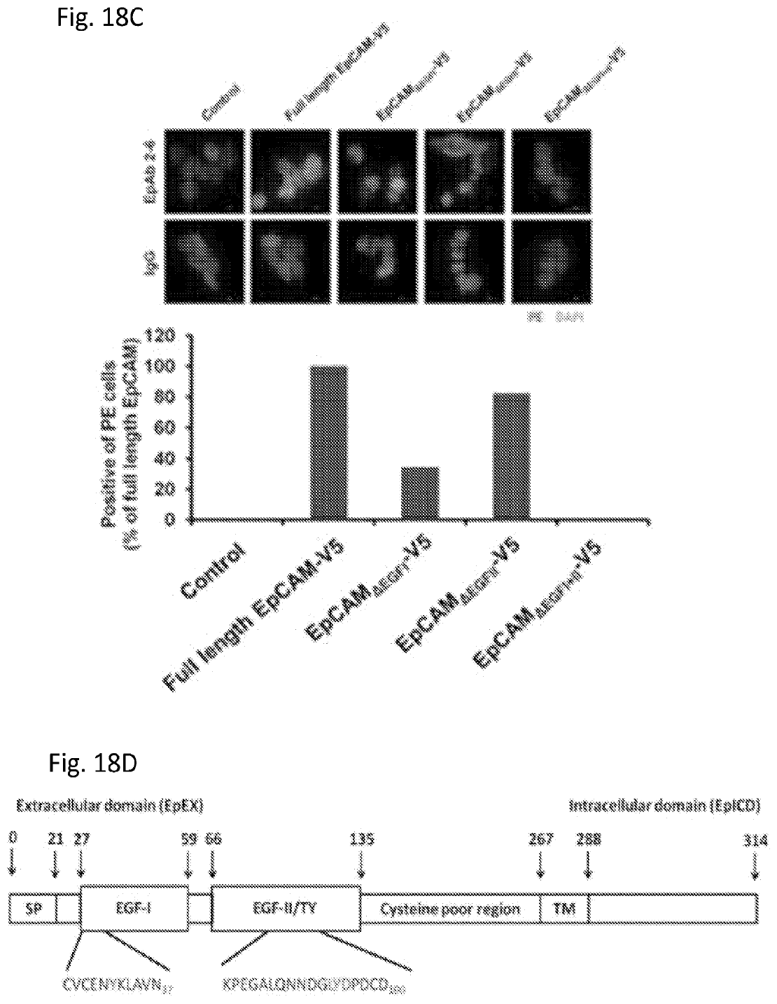

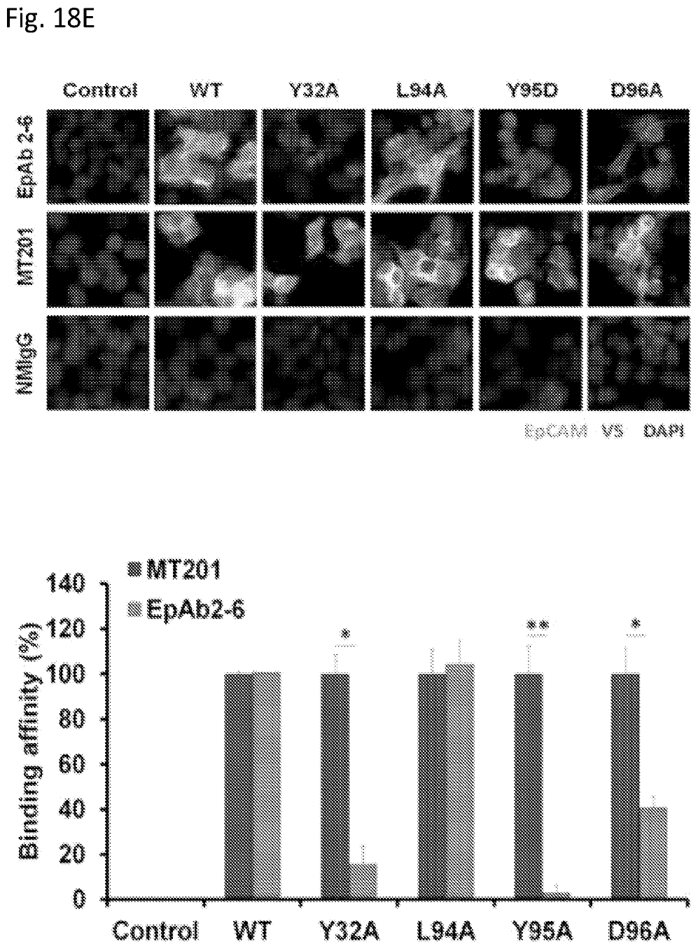

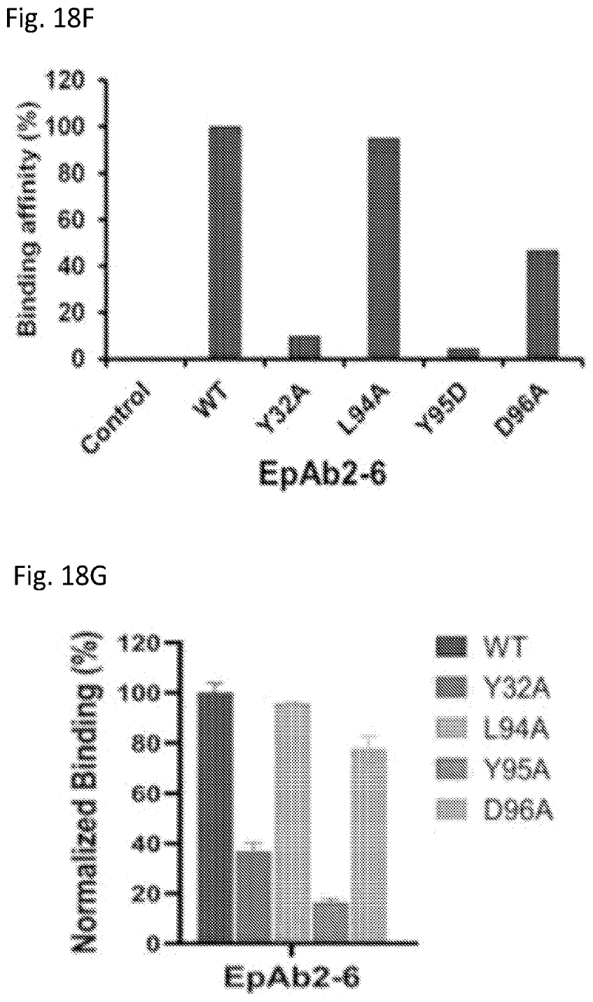

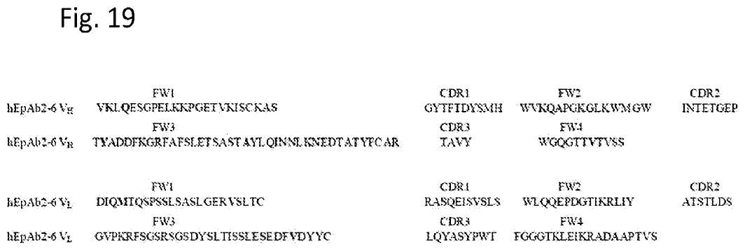

Here, we wanted to determine whether the antibody binds to EpCAM at both EGF-like domains of EpEX (FIG. 18A, FIG. 18B, FIG. 18C). To confirm that EpAb2-6 recognizes the LYD motif in EpCAM, we constructed cDNA sequences encoding the first (aa 27-59; EGF-I domain) and second (aa 66-135; EGF-II/TY domain) EGF-like repeats of EpCAM. PCR-based site-directed mutagenesis was then used to introduce mutations into each domain (FIG. 18D). The reactivity of EpAb2-6 antibody toward these EpCAM mutants was evaluated by immunofluorescence (FIG. 18E), flow cytometry (FIG. 18F), and cellular ELISA (FIG. 18G). Amino acid mutations at EpCAM positions Y32 (EGF-I domain) or Y95 (EGF-II domain) caused marked reductions in EpAb2-6 binding but did not affect MT201 binding. Thus, we conclude that EpAb2-6 binds to the EGF-I and EGF-II domains of EpEX, respectively targeting amino acid residues Y32 and Y95.

### 3. Discussion

EpCAM is known to be a potent CSC surface antigen, and its high expression has been reported as a common feature of CRC (Boesch et al., 2018; Dalerba et al., 2007; Gires et al., 2009; Gires et al., 2020; Lin et al., 2012). In addition to the intracellular effects of EpICD, EpCAM signals via EpEX in the extracellular tumor microenvironment. In this regard, the phenotypes of cancer cells result from their anomalous and heterogeneous cell signaling networks, which may confer self-renewal ability and high tumorigenic potential. Furthermore, certain subpopulations of cancer cells may exhibit the property of stemness that thought to carry robust tumorigenic potential, such that even a single CSC in melanoma may form an entire heterogeneous tumor (Quintana et al., 2008). Because of this malignant potential, ablating CSCs would be highly beneficial when treating cancer patients. However, this goal remains elusive due to the high plasticity of cancer cells, i.e., non-CSCs may de-differentiate to become CSCs when appropriately stimulated by the microenvironment. Ablation of CSCs may therefore require not only direct targeting of the CSC population but also a simultaneous blockade of certain signals from the microenvironment (Batlle and Clevers, 2017). In particular, CRC microenvironments are often enriched in Wnt ligands, which have been shown to confer stemness via D-Catenin signaling (Batlle and Clevers, 2017; Vermeulen et al., 2010; Voloshanenko et al., 2013). In fact, CRC has been modeled to support the contextual functionality of CSCs (Batlle and Clevers, 2017). As such, the crypt niche of intestinal stem cells (ISCs) is enriched with Wnt ligands that serve to maintain the undifferentiated state of the stem cells. Genetic alterations that aberrantly affect Wnt signaling can transmute the crypt-progenitor phenotype to CRC, suggesting ISCs are a major cell type of origin in CRC (Barker et al., 2009; van de Wetering et al., 2002). These studies suggest that Wnt signaling is centrally involved in the function of the CRC niche, as a factor imposing stemness.

Nuclear accumulation of β-Catenin, a hallmark of canonical Wnt pathway, occurs upon Wnt ligands binding to its receptors, recruiting the destruction complex to the cell membrane, thus dephosphorylating β-Catenin, called the active β-Catenin (Nusse and Clevers, 2017). Here, we further showed that, EpEX could also induce nuclear accumulation of β-Catenin via the interaction with Wnt receptors activating the signaling. Therefore, the interaction of either Wnt proteins or EpEX with Wnt receptors can release β-Catenin to form a complex with EpICD that travels to the nucleus where it transcribes EpCAM target genes, such as Wnt receptor proteins and sternness factors (Lin et al., 2012). Under the influence of Wnt or EpEX, β-Catenin may travel to the nucleus independent of EpICD, still allowing TCF/LEF to function as transcription factors for Wnt target genes, such as EpCAM itself and Axin2 (Gires et al., 2020; Maetzel et al., 2009; Nusse and Clevers, 2017). Notably, overproduction of EpEX and EpICD results in hyperactive EpCAM signaling. We previously reported that, stimulation of ERK1/2 signaling via EpEX-EGFR axis might lead to the phosphorylation of TACE and presenilin-2 activating the enzymes enhancing EpEX and EpICD cleavage in CRC and lung cancer (Chen et al., 2020; Liang et al., 2018). Here, we further found Wnt and EpEX proteins also activate TACE and presenilin-2 via Wnt signaling, with the requirements of GSK3 and CK1 establishing a positive feedback-loop. Therefore, functions of EpEX as a ligand for Wnt receptors display as an extrinsic cue in the tumor microenvironment whist EpICD participates in transcription of key Wnt receptor proteins attaining potential cancer sternness.

Currently, therapeutic strategies for cancer are mostly designed to target the disease by eliminating cancer cells via standard anti-proliferative chemotherapy. However, such strategies often suffer from limited positive outcomes. Upon cessation of treatment, some residual cell populations capable of regenerating disease (called chemotherapy-resistant cells) become enriched in CSCs. Disease relapse is often attributable to CSCs that have developed drug resistance via multiple independent mechanisms (Borst, 2012; Holohan et al., 2013). Thus, the inherent abilities of CSCs to exhibit plasticity and quiescence are thought to be robust drivers of drug resistance (Borst, 2012). Intriguingly, CSCs acquire such attributes from extrinsic cues in the microenvironment that include extracellular Wnt machinery (Batlle and Clevers, 2017; Nusse and Clevers, 2017). In fact, attempts have been made to target the Wnt pathway (inhibitors for porcupine, FZD proteins and anti-RSPO3), aiming to restrain CSC signaling; however, these strategies have been thwarted by drug resistance and regeneration of the CSC pool (Batlle and Clevers, 2017; Kahn, 2014). Furthermore, several cancer types including CRC do abundantly express EpCAM (Gires et al., 2020), thus EpEX is enriched functioning as an extrinsic cue in the tumor microenvironment. In order to target a CSC-population as well as CSC-inducing cues, simultaneous targeting both EpCAM and Wnt signaling would be needed that might overcome the drug resistance. Here we show that our anti-EpCAM antibody, EpAb2-6, in combination with LGK974, can attenuate mechanisms in relation to cancer sternness in order to induce apoptosis to cancer cells thus hindering tumor progression in mouse models. Notably, we did not observe significant effects of LGK974 alone, in terms of inducing apoptosis or inhibiting cancer progression in animal models, which is in agreement with previous studies (Cho et al., 2020). However, the inhibitor in combination with EpAb2-6 showed promising therapeutic efficacy. Therefore, these findings will benefit the design of better strategies for CSC therapy as well as may serve to overcome drug resistance.

Both Wnt and EpCAM promote transcription of key genes in cancer progression, proliferation, EMT, metastasis and sternness (Gires et al., 2020; Lin et al., 2012). Moreover, both signaling components contribute to the CSC phenotype and CSC-microenvironment communication in CRC. Interestingly, we found that in the absence of functional Wnt ligands (when cells were treated with LGK974), EpEX sustained β-Catenin signaling and cancer sternness. Only the combined inhibition of Wnt ligands and EpEX was able to completely inhibit Wnt pathway activity and abolish cancer sternness. Therefore, combined treatment with EpAb2-6 and porcupine inhibitors may be an effective strategy to target CSCs. The common features of many cancer types (especially solid tumors) that display high expressions of both EpCAM and Wnt machinery where EpCAM may further stimulate Wnt signaling. Thus, blocking Wnt ligands might not entirely stop the signaling since EpCAM would further activate the pathway, sustaining CSCs and augmenting cancer propagation. In such cases, blocking both EpEX and Wnt ligands would be necessary to repress cancer progression. This blockade of cancer progression could be due to a lack of pro-survival intracellular signaling that contributes to the CSC phenotype as well as inhibition of communication between the microenvironment and tumor cells. The mechanistic insights gained from our study may be useful to improve existing treatments or to develop novel anticancer therapeutics.

### REFERENCES

Barker, N., Ridgway, R. A., van Es, J. H., van de Wetering, M., Begthel, H., van den Born, M., Danenberg. E., Clarke, A. R., Sansom, O. J., and Clevers, H. (2009). Crypt stem cells as the cells-of-origin of intestinal cancer. Nature 457, 608-611.
Batlle, E., and Clevers, H. (2017). Cancer stem cells revisited. Nat Med 23, 1124-1134.
Boesch, M., Spizzo, G., and Seeber, A. (2018). Concise Review: Aggressive Colorectal Cancer: Role of Epithelial Cell Adhesion Molecule in Cancer Stem Cells and Epithelial-to-Mesenchymal Transition. Stem Cells Transl Med 7, 495-501.
Borst, P. (2012). Cancer drug pan-resistance: pumps, cancer stem cells, quiescence, epithelial to mesenchymal transition, blocked cell death pathways, persisters or what?Open Biol 2, 120066. Cancer Genome Atlas, N. (2012). Comprehensive molecular characterization of human colon and rectal cancer. Nature 487, 330-337.
Chen, H. N., Liang, K. H., Lai, J. K., Lan, C. H., Liao, M. Y., Hung, S. H., Chuang, Y. T., Chen, K. C., Tsuei, W. W., and Wu, H. C. (2020). EpCAM Signaling Promotes Tumor Progression and Protein Stability of PD-L1 through the EGFR Pathway. Cancer Res 80, 5035-5050.
Cho, Y. H., Ro, E. J., Yoon, J. S., Mizutani, T., Kang, D. W., Park, J. C., Il Kim, T., Clevers, H., and Choi, K. Y. (2020). 5-FU promotes sternness of colorectal cancer via p53-mediated WNT/beta-catenin pathway activation. Nat Commun 11, 5321.
Dalerba, P., Dylla, S. J., Park, I. K., Liu, R., Wang, X., Cho, R. W., Hoey, T., Gurney, A., Huang, E. H., Simeone, D. M., et al. (2007). Phenotypic characterization of human colorectal cancer stem cells. Proc Natl Acad Sci USA 104. 10158-10163.
de Sousa e Melo, F., Kurtova, A. V., Harnoss, J. M., Kljavin, N., Hoeck, J. D., Hung, J., Anderson, J. E., Storm, E. E., Modrusan, Z., Koeppen, H., et al. (2017). A distinct role for Lgr5(+) stem cells in primary and metastatic colon cancer. Nature 543, 676-680.
Gires, O., Klein, C. A., and Baeuerle, P. A. (2009). On the abundance of EpCAM on cancer stem cells. Nat Rev Cancer 9, 143; author reply 143.
Gires, O., Pan, M., Schinke, H., Canis, M., and Baeuerle, P. A. (2020). Expression and function of epithelial cell adhesion molecule EpCAM: where are we after 40 years?Cancer Metastasis Rev 39, 969-987.
Holohan, C., Van Schaeybroeck, S., Longley, D. B., and Johnston. P. G. (2013). Cancer drug resistance: an evolving paradigm. Nat Rev Cancer 13, 714-726.
Kahn, M. (2014). Can we safely target the WNT pathway?Nat Rev Drug Discov 13, 513-532.
Kim, J. H., Bae, J. M., Song, Y. S., Cho, N. Y., Lee, H. S., and Kang, G. H. (2016). Clinicopathologic, molecular, and prognostic implications of the loss of EPCAM expression in colorectal carcinoma. Oncotarget 7, 13372-13387.
Kozar, S., Morrissey, E., Nicholson, A. M., van der Heijden, M., Zecchini, H. I., Kemp, R., Tavare, S., Vermeulen, L., and Winton, D. J. (2013). Continuous clonal labeling reveals small numbers of functional stem cells in intestinal crypts and adenomas. Cell Stem Cell 13, 626-633.
Liang, K. H., Tso, H. C., Hung, S. H., Kuan, II, Lai, J. K., Ke, F. Y., Chuang, Y. T., Liu, I. J., Wang, Y. P., Chen. R. H., et al. (2018). Extracellular domain of EpCAM enhances tumor progression through EGFR signaling in colon cancer cells. Cancer Lett 433, 165-175.
Liao, M. Y., Lai, J. K., Kuo, M. Y., Lu, R. M., Lin, C. W., Cheng, P. C., Liang, K. H., and Wu, H. C. (2015). An anti-EpCAM antibody EpAb2-6 for the treatment of colon cancer. Oncotarget 6, 24947-24968.
Liao, Y. F., Wang, B. J., Cheng, H. T., Kuo, L. H., and Wolfe, M. S. (2004). Tumor necrosis factor-alpha, interleukin-Ibeta, and interferon-gamma stimulate gamma-secretase-mediated cleavage of amyloid precursor protein through a JNK-dependent MAPK pathway. J Biol Chem 279, 49523-49532.
Lin, C. W., Liao, M. Y., Lin, W. W., Wang, Y. P., Lu, T. Y., and Wu, H. C. (2012). Epithelial cell adhesion molecule regulates tumor initiation and tumorigenesis via activating reprogramming factors and epithelial-mesenchymal transition gene expression in colon cancer. J Biol Chem 287, 39449-39459.
Liu, J., Pan, S., Hsieh, M. H., Ng, N., Sun, F., Wang, T., Kasibhatla, S., Schuller, A. G., Li, A. G., Cheng, D., et al. (2013). Targeting Wnt-driven cancer through the inhibition of Porcupine by LGK974. Proc Natl Acad Sci USA 110, 20224-20229.
MacDonald, B. T., and He, X. (2012). Frizzled and LRP5/6 receptors for Wnt/beta-catenin signaling. Cold Spring Harb Perspect Biol 4.
Maetzel, D., Denzel, S., Mack, B., Canis, M., Went, P., Benk, M., Kieu, C., Papior, P., Baeuerle, P. A., Munz, M., et al. (2009). Nuclear signalling by tumour-associated antigen EpCAM. Nat Cell Biol 11, 162-171.
Morin, P. J., Sparks, A. B., Korinek, V., Barker, N., Clevers, H., Vogelstein, B., and Kinzler, K. W. (1997). Activation of beta-catenin-Tcf signaling in colon cancer by mutations in beta-catenin or APC. Science 275, 1787-1790.
Nusse, R., and Clevers, H. (2017). Wnt/beta-Catenin Signaling, Disease, and Emerging Therapeutic Modalities. Cell 169, 985-999.
Pan, M., Schinke, H., Luxenburger, E., Kranz, G., Shakhtour, J., Libl, D., Huang, Y., Gaber, A., Pavsic, M., Lenarcic, B., et al. (2018). EpCAM ectodomain EpEX is a ligand of EGFR that counteracts EGF-mediated epithelial-mesenchymal transition through modulation of phospho-ERK1/2 in head and neck cancers. PLoS Biol 16, e2006624.
Park, S. Y., Bae, J. S., Cha, E. J., Chu, H. H., Sohn, J. S., and Moon, W. S. (2016). Nuclear EpICD expression and its role in hepatocellular carcinoma. Oncol Rep 36, 197-204.
Quintana, E., Shackleton, M., Sabel, M. S., Fullen, D. R., Johnson, T. M., and Morrison, S. J. (2008). Efficient tumour formation by single human melanoma cells. Nature 456, 593-598.
Ralhan, R., He, H. C., So, A. K., Tripathi, S. C., Kumar, M., Hasan, M. R., Kaur, J., Kashat, L., MacMillan, C., Chauhan, S. S., et al. (2010). Nuclear and cytoplasmic accumulation of Ep-ICD is frequently detected in human epithelial cancers. PLoS One 5, e14130.
Sankpal, N. V., Fleming, T. P., Sharma, P. K., Wiedner, H. J., and Gillanders, W. E. (2017). A double-negative feedback loop between EpCAM and ERK contributes to the regulation of epithelial-mesenchymal transition in cancer. Oncogene 36, 3706-3717.
Schepers, A. G., Snippert, H. J., Stange, D. E., van den Born, M., van Es, J. H., van de Wetering, M., and Clevers, H. (2012). Lineage tracing reveals Lgr5+ stem cell activity in mouse intestinal adenomas. Science 337, 730-735.
Seeber, A., Untergasser, G., Spizzo, G., Terracciano, L., Lugli, A., Kasal, A., Kocher, F., Steiner, N., Mazzoleni, G., Gastl, G., et al. (2016). Predominant expression of truncated EpCAM is associated with a more aggressive phenotype and predicts poor overall survival in colorectal cancer. Int J Cancer 139, 657-663.
van de Wetering, M., Sancho, E., Verweij, C., de Lau, W., Oving, I., Hurlstone, A., van der Horn, K., Batlle, E., Coudreuse, D., Haramis, A. P., et al. (2002). The beta-catenin/TCF-4 complex imposes a crypt progenitor phenotype on colorectal cancer cells. Cell 111, 241-250.
Vermeulen, L., De Sousa, E. M. F., van der Heijden, M., Cameron, K., de Jong, J. H., Borovski, T., Tuynman, J. B., Todaro, M., Merz, C., Rodermond, H., et al. (2010). Wnt activity defines colon cancer stem cells and is regulated by the microenvironment. Nat Cell Biol 12, 468-476.
Voloshanenko, O., Erdmann, G., Dubash, T. D., Augustin, I., Metzig, M., Moffa, G., Hundsrucker, C., Kerr, G., Sandmann, T., Anchang, B., et al. (2013). Wnt secretion is required to maintain high levels of Wnt activity in colon cancer cells. Nat Commun 4, 2610.
Wang, A., Ramjeesingh, R., Chen, C. H., Hurlbut, D., Hammad, N., Mulligan, L. M., Nicol, C., Feilotter, H. E., and Davey, S. (2016). Reduction in membranous immunohistochemical staining for the intracellular domain of epithelial cell adhesion molecule correlates with poor patient outcome in primary colorectal adenocarcinoma. Curr Oncol 23, e171-178.
Wang, Z., Vogelstein, B., and Kinzler, K. W. (2003). Phosphorylation of beta-catenin at S33, S37, or T41 can occur in the absence of phosphorylation at T45 in colon cancer cells. Cancer Res 63, 5234-5235.
Zhan, T., Rindtorff, N., and Boutros, M. (2017). Wnt signaling in cancer. Oncogene 36, 1461-1473.

1. A method for treating cancer, comprising
administering to a subject in need thereof
(i) an effective amount of a first inhibitory agent that inhibits the activation of epithelial cell adhesion molecule (EpCAM) signaling; and
(ii) an effective amount of a second inhibitory agent that inhibits the activation of Wnt signaling.

2. The method of claim 1, wherein the first inhibitory agent reduces production (or release) of an extracellular domain (EpEX) of EpCAM and/or blocks binding of EpEX to a Wnt receptor.

3. The method of claim 1, wherein the second inhibitory agent blocks binding of a Wnt ligand to a Wnt receptor protein.

4. The method of claim 3, wherein the Wnt ligand is not EpEX.

5. The method of claim 1, wherein the first inhibitory agent is an antibody directed to EpEX or an antigen-binding fragment thereof.

6. The method of claim 5, wherein the antibody specifically binds to epidermal growth factor (EGF)-like domains I and II.

7. The method of claim 5, wherein the antibody has a specific binding affinity to an epitope within the sequence of CVCENYKLAVN (aa 27 to 37) (SEQ ID NO: 20) located in the EGF-like domain I, and KPEGALQNNDGLYDPDCD (aa 83 to 100) (SEQ ID NO: 19) located in the EGF-like domain II.

8. The method of claim 5, wherein the antibody or antigen-binding fragment comprises
(a) a heavy chain variable region (VH) which comprises a heavy chain complementary determining region 1 (HC CDR1) comprising the amino acid sequence of SEQ ID NO: 2, a heavy chain complementary determining region 2 (HC CDR2) comprising the amino acid sequence of SEQ ID NO: 4, and a heavy chain complementary determining region 3 (HC CDR3) comprising the amino acid sequence of SEQ ID NO: 6; and
(b) a light chain variable region (VL) which comprises a light chain complementary determining region 1 (LC CDR1) comprising the amino acid sequence of SEQ ID NO: 9, a light chain complementary determining region (LC CDR2) comprising the amino acid sequence of SEQ ID NO: 11, and a light chain complementary determining region 3 (LC CDR3) comprising the amino acid sequence of SEQ ID NO: 13.

9. The method of claim 1, wherein the first inhibitory agent is effective in inhibiting β-Catenin signaling.

10. The method of claim 1, wherein the second inhibitory agent is a porcupine inhibitor.

11. The method of claim 1, wherein the method is effective in inducing apoptosis of cancer cells.

12. The method of claim 1, wherein the method is effective in inhibiting cancer stemness properties, tumor progression and/or metastasis.

13. The method of claim 1, wherein the method is effective in prolonging survival of the subject.

14. The method of claim 1, wherein the cancer is selected from the group consisting of lung cancer, brain cancer, breast cancer, cervical cancer, colon cancer, gastric cancer, head and neck cancer, kidney cancer, leukemia, liver cancer, ovarian cancer, pancreatic cancer, prostate cancer, skin cancer and testicular cancer.

15. A kit or a pharmaceutical composition comprising:
(i) a first inhibitory agent that inhibits the activation of EpCAM signaling; and
(ii) a second inhibitory agent that inhibits the activation of Wnt signaling.

16. The kit or pharmaceutical composition of claim 15, wherein
the first inhibitory agent reduces production (or release) of an extracellular domain (EpEX) of EpCAM and/or blocks binding of EpEX to a Wnt receptor and
the second inhibitory agent blocks binding of a Wnt ligand to a Wnt receptor protein.

17-22. (canceled)
